(19) United States
(12) Patent Application Publication
(10) Pub. No.: US 2026/0144952 A1
(43) Pub. Date: May 28, 2026

(54) PATIENT INTERFACE

(71) Applicant: ResMed Pty Ltd, Bella Vista, New South Wales (AU)

(72) Inventors: Michiel KOOIJ, Sydney (AU); Vinay MANJUNATH, Sydney (AU); Rupert Christian SCHEINER, Sydney (AU); Chiew Khuen WONG, Sydney (AU)

(21) Appl. No.: 19/121,773

(22) PCT Filed: Oct. 19, 2023
(86) PCT No.: PCT/AU2023/051039

§ 371 Date: Apr. 16, 2025

(30) Foreign Application Priority Data
Oct. 20, 2022 (AU) 2022903105
Jun. 29, 2023 (AU) 2023902063
Aug. 18, 2023 (AU) 2023902625

(51) Int. Cl. A61M 16/06

(52) U.S. Cl. CPC A61M 16/0622; A61M 2202/0007; A61M 2202/02; A61M 2205/3344; A61M 2209/088; A61M 2210/0606; A61M 2210/0618; A61M 2210/0625

(57) ABSTRACT

A cushion module comprising a plenum chamber pressurisable to a therapeutic pressure, a seal-forming structure constructed and arranged to form a seal with a region of the patient's face surrounding an entrance to the patient's airways, a fascia portion at least partially forming an anterior side of the cushion module, the seal-forming structure being attached to the fascia portion, wherein the fascia portion comprises a curved shape and is curved at least partially towards a posterior direction on lateral sides of the fascia portion in use, and wherein the fascia portion is biased away from the curved shape towards a flatter shape than the curved shape to provide tautness to the seal-forming structure in use.

### 1 CROSS-REFERENCE TO RELATED APPLICATIONS

This application claims the benefit of, and priority to, Australian Provisional Patent Application No. 2022903105, filed on 20 Oct. 2022, Australian Provisional Patent Application No. 2023902063, filed on 29 Jun. 2023 and Australian Provisional Patent Application No. 2023902625, filed on 18 Aug. 2023, each of which are hereby incorporated by reference herein in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time, or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

Some patient interfaces of the prior art comprise a cushion module having a rigid shell with a predefined shape based on the anthropometrics of a notional person in the middle of a selected size range. As a result, the seal and comfort success with a given size of cushion module may be strongly related to the correlation between the patient's anthropometrics and those of the notional person on which the design is based. This may result in the need to “fit” the patient (possibly with assistance from a suitably qualified professional) to a particular size of cushion module and/or a particular type of interface. This may also result in the need to manufacture a range of different size cushion modules to ensure that a broad range of patients can find a size with suits them.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material, for example silicone, positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. or one or more of its related companies has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

Many of the seal forming structures of the prior art comprise an element made from silicone (or another similar polymer) which creates a seal against the patient's face. However, some patients may dislike the surface texture of silicone and/or its lack of breathability.

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives-see for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 2.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air. Humidifiers therefore often have the capacity to heat the flow of air was well as humidifying it.

### 2.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One aspect of one form of the present technology comprises a patient interface comprising:
a cushion module forming a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure partially forming the cushion module, the seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a vent to allow a continuous flow of gases exhaled by the patient from an interior of the plenum chamber to ambient, said vent being sized and shaped to maintain the therapeutic pressure in the plenum chamber in use;
a fascia portion at least partially forming an anterior side of the cushion module, the seal-forming structure being attached to the fascia portion;
wherein the fascia portion comprises a curved shape and is curved at least partially towards a posterior direction on lateral sides of the fascia portion in use, and wherein the fascia portion is biased away from the curved shape towards a flatter shape than the curved shape to provide tautness to the seal-forming structure in use;
wherein the patient interface is configured to allow the patient to breathe from ambient through their mouth in the absence of a flow of pressurised air through the plenum chamber inlet port, or the patient interface is configured to leave the patient's mouth uncovered in use.

Another aspect of the present technology comprises a cushion module for a patient interface, the cushion module comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a fascia portion at least partially forming an anterior side of the cushion module, the seal-forming structure being attached to the fascia portion;
wherein the fascia portion comprises a curved shape and is curved at least partially towards a posterior direction on lateral sides of the fascia portion in use, and wherein the fascia portion is biased away from the curved shape towards a flatter shape than the curved shape to provide tautness to the seal-forming structure in use.

In examples:
the fascia portion is first formed into a formed shape having less curvature than the curved shape, and wherein after attachment of the seal-forming structure to the fascia portion the seal-forming structure holds the fascia portion in the curved shape, and the fascia portion is biased away from the curved shape by a tendency to return to the formed shape;
the fascia portion is thermoformed into the formed shape;
the cushion module comprises a resilient member biasing the fascia portion away from the curved shape;
the resilient member is provided interior to the plenum chamber;
the resilient member engages the fascia portion along a connection between the fascia portion and the seal-forming structure;
the resilient member is biased towards an annular shape;
the resilient member is formed in the annular shape and deformed during provision to the interior of the plenum chamber, the resilient member being biased towards the annular shape in use to bias the fascia portion away from the curved shape in use;
the resilient member is formed from a polymer material; and/or
the resilient member is moulded.

In further examples:
the fascia portion at least partially forms the plenum chamber;
the fascia portion is formed at least partially from one or more foam and/or textile materials;
the fascia portion is formed from foam covered with a textile material on at least a non-patient facing side thereof;
the fascia portion has a non-zero negative first principal curvature and a second principal curvature which is less than the first principal curvature;
the seal-forming structure comprises a membrane portion configured to engage the patient's face;
the membrane portion is configured to form a seal to at least a pronasale region and the nasal alae of the patient's nose;
the membrane portion comprises a nasal hole through which air can flow to both the patient's nares, in use;
the membrane portion comprises an oral hole through which air can flow to the patient's mouth, in use;
the membrane portion is at least partially formed from a textile material and is air-impermeable;
the cushion module comprises at least one pair of headgear connection portions connected to the fascia portion and configured to connect to a positioning and stabilising structure;
the at least one pair of headgear connection portions comprises a pair of superior headgear connection portions connected to the fascia portion and a pair of inferior headgear connection portions connected to the fascia portion;
each of the superior headgear connection portions comprises a curved arm; and/or
each of the inferior headgear connection portions comprises a magnetic connector.

Another aspect of one form of the present technology is a patient interface comprising:
a cushion module forming a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure partially forming the cushion module, the seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a vent to allow a continuous flow of gases exhaled by the patient from an interior of the plenum chamber to ambient, said vent being sized and shaped to maintain the therapeutic pressure in the plenum chamber in use;
a fascia portion at least partially forming an anterior side of the cushion module, the seal-forming structure being attached to the fascia portion;
wherein the seal-forming structure comprises:
   a membrane portion configured to contact the patient's face in use, the membrane portion being attached to a periphery of the fascia portion;
   a pair of seal support portions, each provided to a respective lateral side of the fascia portion and projecting in an at least partially medial direction from the fascia portion, each seal support portion configured to urge the membrane portion against the patient's face in use;
wherein the patient interface is configured to allow the patient to breathe from ambient through their mouth in the absence of a flow of pressurised air through the plenum chamber inlet port, or the patient interface is configured to leave the patient's mouth uncovered in use.

Another aspect of the present technology comprises a cushion module for a patient interface, the cushion module comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a fascia portion at least partially forming an anterior side of the cushion module, the seal-forming structure being attached to the fascia portion;
wherein the seal-forming structure comprises:
   a membrane portion configured to contact the patient's face in use, the membrane portion being attached to a periphery of the fascia portion; and
   a pair of seal support portions, each provided to a respective lateral side of the fascia portion and projecting in an at least partially medial direction from the fascia portion, each seal support portion configured to urge the membrane portion against the patient's face in use.

In examples:
each of the pair of seal support portions projects from at or proximate the periphery of the fascia portion;
each of the pair of seal support portions projects from proximate a peripheral edge of the fascia portion but spaced from the peripheral edge by a spacing;
the spacing is within the range of 0.5-5 mm;
the spacing is within the range of 1-3 mm;
the membrane portion comprises a nasal hole through which air can flow to both of the patient's nares, in use;
the membrane portion comprises an oral hole through which air can flow to the patient's mouth, in use;
each of the pair of seal support portions is structured to urge the membrane portion against a respective one of the patient's cheeks;
the membrane portion is configured to form a seal to at least a pronasale region and the nasal alae of the patient's nose;
each of the pair of seal support portions is structured and arranged to urge the membrane portion to seal at or proximate a respective one of the alar crest points on the patient's face;
each of the pair of seal support portions comprises a medial projection structured and arranged to urge the membrane portion towards the user's face proximate a respective one of the alar crest points;
each of the pair of seal support portions comprises an inferior portion having a concave medial edge;
each of the pair of seal support portions comprises a superior portion having a convex medial edge;
each seal support portion is stiffer in the inferior portion than in the superior portion;
each of the pair of seal support portions comprises a superior portion having a concave medial edge structured and arranged to lie proximate to and follow the shape of the patient nasal ala;
each of the pair of seal support portions comprises a superior portion and an inferior portion, each seal support portion being stiffer in the inferior portion than in the superior portion;
the membrane portion overhangs each seal support portion;
the fascia portion at least partially forms the plenum chamber;
the fascia portion is formed at least partially from one or more foam and/or textile materials;
the fascia portion is formed from foam covered with a textile material on at least a non-patient facing side thereof;
the seal support portions are at least partially formed from foam;
the seal support portions are formed from the same material as the fascia portion;
the fascia portion comprises a curved shape and is curved at least partially towards a posterior direction on lateral sides of the fascia portion in use;
wherein the fascia portion is thermoformed to shape; and/or
wherein the seal support portions are not thermoformed to shape.

In further examples:
the fascia portion has a non-zero negative first principal curvature and a second principal curvature which is less than the first principal curvature;
the membrane portion is at least partially formed from a textile material and is air-impermeable;
the cushion module comprises at least one pair of headgear connection portions connected to the fascia portion and configured to connect to a positioning and stabilising structure;
the at least one pair of headgear connection portions comprises a pair of superior headgear connection portions connected to the fascia portion and a pair of inferior headgear connection portions connected to the fascia portion;
each of the superior headgear connection comprises a curved arm; and/or
each of the inferior headgear connection comprises a magnetic connector.

Another aspect of one form of the present technology is a patient interface comprising:
a cushion module forming a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure at least partially forming the cushion module, the seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a vent to allow a continuous flow of gases exhaled by the patient from an interior of the plenum chamber to ambient, said vent being sized and shaped to maintain the therapeutic pressure in the plenum chamber in use;
a fascia portion formed from a flexible material and at least partially forming an anterior side of the cushion module, the seal-forming structure being attached to the fascia portion;
a pair of first headgear connection portions configured to connect to first strap portions of a positioning and stabilising structure, the first headgear connection portions each being formed from a flexible material and each being attached to a non-patient facing side of the fascia portion proximate lateral sides of the fascia portion;
wherein the fascia portion comprises a peripheral edge formed by a superior edge portion and an inferior edge portion connected to each other by a pair of lateral edge portions at respective lateral sides of the fascia portion, each lateral edge portion comprising a lateralmost point, wherein each of the first headgear connection portions is attached to the fascia portion medially of the lateral edge portion;
wherein the patient interface is configured to allow the patient to breathe from ambient through their mouth in the absence of a flow of pressurised air through the plenum chamber inlet port, or the patient interface is configured to leave the patient's mouth uncovered in use.

Another aspect of the present technology comprises a cushion module for a patient interface, the cushion module comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a fascia portion formed from a flexible material and at least partially forming an anterior side of the cushion module, the seal-forming structure being attached to the fascia portion;
a pair of first headgear connection portions configured to connect to first strap portions of a positioning and stabilising structure, the first headgear connection portions each being formed from a flexible material and each being attached to a non-patient facing side of the fascia portion proximate lateral sides of the fascia portion; and
wherein the fascia portion comprises a peripheral edge formed by a superior edge portion and an inferior edge portion connected to each other by a pair of lateral edge portions at respective lateral sides of the fascia portion, each lateral edge portion comprising a lateralmost point, wherein each of the first headgear connection portions is attached to the fascia portion medially of the lateral edge portion.

In examples:
the first headgear connection portions are structured and arranged to urge the fascia portion proximate the lateralmost point towards the patient's face in use;
each of the first headgear connection portions is attached to the fascia portion proximate both the superior edge portion and the inferior edge portion of the peripheral edge of the fascia portion;
each of the first headgear connection portions is attached to the fascia portion at a joint lying along the non-patient facing side of the fascia portion from at or proximate the superior edge portion to at or proximate the inferior edge portion of the peripheral edge of the fascia portion;
each of the first headgear connection portions is able to pivot with respect to the fascia portion along the joint;
each of the first headgear connection portions comprises a superior edge, an inferior edge and a first strap connection point configured to attach to the first strap portion of the positioning and stabilising structure, the superior edge and inferior edge of each of the first headgear connection portions converging towards each other towards the first strap connection point;
the superior edge of each of the first headgear connection portions lies in use substantially tangential to the superior edge portion of the peripheral edge of the fascia portion;
the inferior edge of each of the first headgear connection portions lies in use substantially tangential to the inferior edge portion of the peripheral edge of the fascia portion;
each of the first headgear connection portions is formed from a textile material;
at least a central portion of each of the first headgear connection portions is formed from a webbing;
the first headgear connection portions are superior headgear connection portions, the first strap connection points are superior strap connection points and the first strap portion of the positioning and stabilising structure is a superior strap portion, and the cushion module further comprises a pair of inferior headgear connection portions configured to connect to inferior strap portions of the positioning and stabilising structure;
each inferior headgear connection portion comprises a magnetic headgear connection point provided to the fascia portion to which a respective inferior strap portion is able to magnetically attach;
the fascia portion is at least partially formed from a foam and/or textile material;
the fascia portion is formed from foam covered with a textile material on at least a non-patient facing side thereof;
the cushion module comprises a deformable adjustment member attached to the fascia portion, structured and arranged to be selectively adjusted by the patient to adjust a curvature of the fascia portion;
the deformable adjustment member comprises a deformable metal strip spanning from one lateral side of the fascia portion to the other lateral side; and/or
the deformable metal strip spans between locations at or proximate the joints between the first headgear connection portions and the fascia portion.

In further examples:
the fascia portion has a non-zero negative first principal curvature and a second principal curvature which is less than the first principal curvature;
the seal-forming structure comprises a membrane portion configured to engage the patient's face;
the membrane portion is configured to form a seal to at least a pronasale region and the nasal alae of the patient's nose;
the membrane portion comprises a nasal hole through which air can flow to both the patient's nares, in use;
the membrane portion comprises an oral hole through which air can flow to the patient's mouth, in use; and/or
the membrane portion is at least partially formed from a textile material and is air-impermeable.

Another aspect of one form of the present technology is a patient interface comprising:
a cushion module forming a plenum chamber pressurisable to a therapeutic pressure of at least 4 cmH2O above ambient air pressure, said plenum chamber including a plenum chamber inlet port sized and structured to receive a flow of air at the therapeutic pressure for breathing by a patient;
a seal-forming structure at least partially forming the cushion module, the seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use, the seal-forming structure comprising at least one nasal hole through which air can flow to the patient's nasal airways and at least one oral hole through which air can flow to the patient's mouth;
a vent to allow a continuous flow of gases exhaled by the patient from an interior of the plenum chamber to ambient, said vent being sized and shaped to maintain the therapeutic pressure in the plenum chamber in use;
a fascia portion formed from a flexible material and at least partially forming an anterior side of the cushion module, the seal-forming structure being attached to the fascia portion;
a pair of lateral headgear connection portions extending posteriorly of the seal-forming structure at lateral sides of the cushion module, the lateral headgear connection portions configured to connect to lateral strap portions of a positioning and stabilising structure, the lateral headgear connection portions each being formed from a flexible material;
wherein the cushion module is configured to be supported in position on the patient's face in use by the lateral strap portions of the positioning and stabilising structure in the absence of any other strap portions of the positioning and stabilising structure;
wherein the patient interface is configured to allow the patient to breathe from ambient through their mouth in the absence of a flow of pressurised air through the plenum chamber inlet port.

Another aspect of the present technology comprises a cushion module for a patient interface, the cushion module comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a fascia portion formed from a flexible material and at least partially forming an anterior side of the cushion module, the seal-forming structure being attached to the fascia portion; and
a pair of lateral headgear connection portions extending posteriorly of the seal-forming structure at lateral sides of the cushion module, the lateral headgear connection portions configured to connect to lateral strap portions of a positioning and stabilising structure, the lateral headgear connection portions each being formed from a flexible material;
wherein the cushion module is configured to be supported in position on the patient's face in use by the lateral strap portions of the positioning and stabilising structure in the absence of any other strap portions of the positioning and stabilising structure.

In examples:
the fascia portion comprises a superior edge portion and an inferior edge portion defining a height of the fascia portion, and each of the lateral headgear connection portions comprises a superior edge and an inferior edge defining a height of the respective lateral headgear connection portion;
each of the lateral headgear connection portions is attached to the fascia portion proximate both the superior edge portion and the inferior edge portion of the peripheral edge of the fascia portion;
the height of each lateral headgear connection portion being at least half the height of the fascia portion;
the height of each lateral headgear connection portion is at least two-thirds the height of the fascia portion;
the superior edges of the respective lateral headgear connection portions are located proximate the superior edge portion of the fascia portion;
the inferior edges of the respective lateral headgear connection portions are located proximate the inferior edge portion of the fascia portion;
the height each lateral headgear connection portion is substantially the same as the height of the fascia portion;
each of the lateral headgear connection portions is connected to the fascia portion substantially continuously along the height of the respective lateral headgear connection portion;
each of the lateral headgear connection portions is able to pivot laterally and medially with respect to the fascia portion;
each of the lateral headgear connection portions comprises a slot to which the respective lateral strap portion of the positioning and stabilising structure is able to connect in use;
the lateral headgear connection portions are integrally formed with the fascia portion;
the fascia portion is formed from a foam and/or textile material;
each of the lateral headgear connection portions is formed from a foam and/or textile material; and/or
the fascia portion is formed from foam covered with a textile material on at least a non-patient facing side thereof.

In further examples:
the fascia portion has a non-zero negative first principal curvature and a second principal curvature which is less than the first principal curvature;
the seal-forming structure comprises a membrane portion configured to engage the patient's face;
the membrane portion is configured to form a seal to at least a pronasale region and the nasal alae of the patient's nose; and/or
the membrane portion is at least partially formed from a textile material and is air-impermeable.

Another aspect of one form of the present technology is a patient interface comprising:
a cushion module forming a plenum chamber pressurisable to a therapeutic pressure of at least 4 cmH2O above ambient air pressure, said plenum chamber including a plenum chamber inlet port sized and structured to receive a flow of air at the therapeutic pressure for breathing by a patient;
a seal-forming structure at least partially forming the cushion module, the seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a vent to allow a continuous flow of gases exhaled by the patient from an interior of the plenum chamber to ambient, said vent being sized and shaped to maintain the therapeutic pressure in the plenum chamber in use;
a fascia portion formed from a flexible material and at least partially forming an anterior side of the cushion module, the seal-forming structure being attached to the fascia portion;
a frame attached to the fascia portion, the frame constructed and arranged to rigidise the fascia portion, the frame having a pair of lateral headgear connection portions located on respective lateral sides of the cushion module, the lateral headgear connection portions configured to connect to lateral strap portions of a positioning and stabilising structure;
wherein the patient interface is configured to allow the patient to breathe from ambient through their mouth in the absence of a flow of pressurised air through the plenum chamber inlet port, or the patient interface is configured to leave the patient's mouth uncovered in use.

Another aspect of the present technology comprises a cushion module for a patient interface, the cushion module comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a fascia portion formed from a flexible material and at least partially forming an anterior side of the cushion module, the seal-forming structure being attached to the fascia portion; and
a frame attached to the fascia portion, the frame constructed and arranged to rigidise the fascia portion, the frame having a pair of lateral headgear connection portions located on respective lateral sides of the cushion module, the lateral headgear connection portions configured to connect to lateral strap portions of a positioning and stabilising structure.

In examples:
the cushion module is configured to be supported in position on the patient's face in use by the lateral strap portions of the positioning and stabilising structure in the absence of any other strap portions of the positioning and stabilising structure;
the frame is in the form of a skeleton;
the frame is formed by a plurality of elongate portions connected to each other;
the frame is constructed and arranged to impart an in-use shape to the fascia portion;
the fascia portion comprises a peripheral edge formed by a superior edge portion and an inferior edge portion connected to each other by a pair of lateral edge portions at respective lateral sides of the fascia portion, and the frame comprises a peripheral portion having a shape corresponding to the peripheral edge of the fascia portion;
the shape of the peripheral portion of the frame substantially follows the peripheral edge of the fascia portion;
each lateral strap connection portion of the frame connects between a superior portion of the frame and an inferior portion of the frame;
the superior edge portion of the fascia portion curves posteriorly away from the mid-sagittal plane on either lateral side of the patient's face in use, the lateral edge portions curve posteriorly and inferiorly away from the superior edge portions and then inferiorly and anteriorly towards the inferior edge portion, and the inferior edge portion curves medially back towards the mid-sagittal plane,
each lateral strap connection portion connects to the superior portion of the frame proximate a junction between the superior edge portion and a respective lateral edge portion of the peripheral edge of the fascia portion;
each lateral strap connection portion connects to the inferior portion of the frame proximate a junction between the inferior edge portion and respective lateral edge portion of the peripheral edge of the fascia portion;
each lateral strap connection portion comprises an elongate portion around which a respective lateral strap portion of the positioning and stabilising structure is able to wrap and be secured back to itself;
the fascia portion is formed from a foam and/or textile material;
the fascia portion is formed from foam covered with a textile material on at least a non-patient facing side thereof;
the fascia portion comprises lateral pockets structured and arranged to receive and retain the frame in use;
the lateral pockets are formed from textile material;
the frame comprises a pair of lateral portions, each being lateral of a respective one of the lateral strap connection portions, each lateral portion being received in a respective one of the lateral pockets in use.

In further examples:
the fascia portion has a non-zero negative first principal curvature and a second principal curvature which is less than the first principal curvature;
the seal-forming structure comprises a membrane portion configured to engage the patient's face;
the membrane portion is configured to form a seal to at least a pronasale region and the nasal alae of the patient's nose;
the membrane portion comprises a nasal hole through which air can flow to both the patient's nares, in use;
the membrane portion comprises an oral hole through which air can flow to the patient's mouth, in use; and/or
the membrane portion is at least partially formed from a textile material and is air-impermeable.

Another aspect of one form of the present technology is to provide a patient interface that can flex to accommodate patients having faces of varying widths.

Another aspect of one form of the present technology is to provide an oro-nasal patient interface that is lightweight.

Another aspect of one form of the present technology is to provide an oro-nasal patient interface that is comfortable and low cost.

Another form of the present technology comprises a patient interface comprising:
a plenum chamber pressurisable to a therapeutic pressure of at least 6 cmH2O above ambient air pressure, the plenum chamber having at least one plenum chamber inlet port sized and structured to receive a flow of air at the therapeutic pressure for breathing by the patient;
at least one pair of headgear connection portions connected to the fascia portion and configured to connect to a positioning and stabilising structure;
a seal-forming structure and partially forming the plenum chamber, the seal-forming structure being constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
a fascia portion at least partially forming an anterior side of the patient interface, the seal-forming structure being attached to the fascia portion;
wherein the seal forming structure comprises an undercushion, and a membrane portion configured to form a seal in use to at least a pronasale region and nasal alae of the patient's nose, the seal forming structure further configured to form a seal around the patient's mouth;
wherein the fascia portion is at least partially formed from a foam and/or textile material, the undercushion is attached to a patient-facing surface of the fascia portion, and the membrane portion substantially covers a patient-facing side of the undercushion, the membrane portion being attached to the undercushion and/or the fascia portion.

In examples:
wherein the fascia portion at least partially forms the plenum chamber;
the undercushion and membrane portion form substantially all of the plenum chamber;
the fascia portion is formed from foam covered with a textile material on a non-patient-facing surface of the fascia portion;
the fascia portion comprises a textile layer on the patient-facing surface;
the fascia portion is formed from a textile material and one or more air-impermeable layers;
the undercushion is formed from foam;
the undercushion is formed from a polyurethane foam, for example a thermoplastic polyurethane;
the undercushion is air-impermeable;
the fascia portion comprises a curved three-dimensional shape in use;
the fascia portion is thermoformed into the curved three-dimensional shape so as to support itself in the curved three-dimensional shape;
the undercushion is formed from compression cut foam;
the undercushion is formed from moulded foam;
the fascia portion is unable to support itself in the curved three-dimensional shape; and/or
the undercushion is formed from moulded foam and supports the fascia portion in the curved three-dimensional shape.

In further examples:
the undercushion is configured to hold the membrane portion substantially taut when the patient interface is not in use;
the membrane portion is bonded to the undercushion and/or the fascia portion;
the membrane portion comprises a first hole through which air can flow to both the patient's nares, in use;
the membrane portion comprises a second hole through which air can flow to the patient's mouth, in use;
the membrane portion is at least partially formed from a textile material and is air-impermeable;
the undercushion comprises a nasal portion configured to be positioned proximate an inferior periphery of the patient's nose in use, the undercushion forming a nasal recess in the nasal portion comprising a shape corresponding to the inferior periphery of the patient's nose; and/or
the membrane portion is attached to the undercushion around the periphery of the nasal recess.

In further examples:
the fascia portion has a non-zero negative first principal curvature and a second principal curvature which is less than the first principal curvature;
the second principal curvature is substantially zero and substantially parallel, in use, to the patient's sagittal plane;
the fascia portion is configured to flex such that the first principal curvature has a larger magnitude when donned by a patient with a narrow face than when donned by a patient having a relatively wider face;
the at least one pair of headgear connector portions comprises a pair of superior headgear connector portions connected to the chassis portion and a pair of inferior headgear connector portions connected to the chassis portion;
each of the superior headgear connectors comprises a buckle provided to a respective end of a band; and/or
each of the inferior headgear connector portions comprises a magnetic connector.

Another form of the present technology comprises a patient interface comprising:
a plenum chamber pressurisable to a therapeutic pressure of at least 6 cmH2O above ambient air pressure, the plenum chamber having at least one plenum chamber inlet port sized and structured to receive the flow of air at the therapeutic pressure for breathing by the patient;
at least one pair of headgear connector portions configured to connect to a positioning and stabilising structure;
a seal-forming structure partially forming the plenum chamber, the seal-forming structure being constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
wherein the seal forming structure comprises an undercushion and a membrane portion connected to the undercushion, the membrane portion configured to form a seal to at least a pronasale region and the nasal alae of the patient's nose, the seal forming structure further configured to form a seal around the patient's mouth;
wherein the undercushion comprises a nasal portion configured to be positioned proximate an inferior periphery of the patient's nose in use, the undercushion forming a nasal recess in the nasal portion configured to at least partially surround the inferior periphery of the patient's nose in use, the membrane portion being supported by the undercushion at a periphery of the nasal recess, wherein the nasal portion of the undercushion comprises:
   a medial peripheral portion partially defining the periphery of the nasal recess and structured to support the membrane portion at a medial position proximate the patient's pronasale;
   a pair of lateral peripheral portions partially defining the periphery of the nasal recess and located on respective lateral sides of the medial peripheral portion, the lateral peripheral portions structured to support the membrane portion superiorly of the patient's nasal alae on respective lateral sides of the patient's nose and superiorly of the medial position at which the medial peripheral portion supports the membrane portion.

In examples:
the lateral peripheral portions curve superiorly along the periphery of the nasal recess away from the medial peripheral portion to respective superior-most points of each lateral peripheral portion;
the lateral peripheral portions curve inferiorly along the periphery of the nasal recess away from the respective superior-most points in a posterior direction;
the medial peripheral portion curves superiorly on either lateral side thereof into the lateral peripheral portions;
each lateral peripheral portion comprises a dome shape at the respective superior-most point;
the medial peripheral portion comprises a saddle shape;
the patient interface comprises a fascia portion at least partially defining the plenum chamber, the undercushion being attached to a patient-facing side of the fascia portion;
each of the lateral peripheral portions extends superiorly of a superior edge of the fascia portion;
the lateral peripheral portions are flexible and structured to deform medially towards the patient's nose in use; and/or
the lateral peripheral portions are structured to bend medially towards the patient's nose in use.

In further examples:
the nasal recess is configured to avoid engaging both lateral sides of the patient's nose simultaneously;
when the patient's nose is centred within the nasal recess, the nasal recess substantially does not engage either lateral side of the patient's nose;
the nasal recess comprises a shape, in a plane parallel to the Frankfort horizontal plane of the patient's head, corresponding to the inferior periphery of the patient's nose;
the nasal recess surrounds substantially all laterally- and anteriorly-facing portions of the inferior periphery of the patient's nose in use;
the nasal recess comprises an inwardly-facing wall at least partially surrounding the inferior periphery of the patient's nose in use;
the inwardly-facing wall faces partially inwardly and partially superiorly;
the inwardly-facing wall extends from at or proximate one of the patient's cheeks around the patient's nose to at or proximate the other of the patient's cheeks;
the membrane portion is attached to the undercushion around the periphery of the nasal recess;
the inwardly-facing wall has a concave cross-section in a plane parallel with the Frankfort horizontal plane of the patient's head;
the inwardly-facing wall has a concave cross-section in the mid-sagittal plane of the patient's head and/or in a plane parallel to the mid-coronal plane of the patient's head;
the undercushion comprises a pair of posterior support portions each positioned on a respective lateral side of the nasal recess and configured to engage the patient's face medially of and proximate to the nasolabial sulci of the patient's face;
the posterior support portions engage the patient's face at locations inferior to the nasal alae on either lateral side of the patient's lip superior and/or at locations aligned vertically with the patient's nasal alae between the nasal alae and the nasolabial sulci;
the posterior support portions are shaped to protrude at least partially medially into concavities formed on the patient's face on either lateral side of the patient's nasal alae; and/or
the inwardly-facing wall extends from a first one of the posterior support portions around the patient's nose to the other one of the posterior support portions.

In further examples:
the undercushion is configured to hold the membrane portion substantially taut when the patient interface is not in use;
the membrane portion is attached to the undercushion around an outer periphery of the membrane portion;
the membrane portion is bonded to the undercushion and/or the fascia portion;
the membrane portion comprises a first hole through which air can flow to both the patient's nares, in use;
the membrane portion comprises a second hole through which air can flow to the patient's mouth, in use;
the membrane portion is at least partially formed from a textile material;
the undercushion is formed from foam;
the undercushion is formed from moulded foam;
the undercushion is formed from compression cut foam;
the undercushion is formed from a polyurethane, for example a thermoplastic polyurethane;
the fascia portion has a non-zero negative first principal curvature and a second principal curvature which is less than the first principal curvature;
the second principal curvature is substantially zero and substantially parallel, in use, to the patient's sagittal plane;
the fascia portion is configured to flex such that the first principal curvature has a larger magnitude when donned by a patient with a narrow face than when donned by a patient having a relatively wider face;
the at least one pair of headgear connectors comprises a pair of superior headgear connectors connected to the chassis portion and a pair of inferior headgear connectors connected to the chassis portion;
each of the superior headgear connectors is formed by a buckle provided to a textile band; and/or
each of the inferior headgear connectors comprises a magnetic connector.

Another form of the present technology comprises a patient interface comprising:
a plenum chamber pressurisable to a therapeutic pressure of at least 6 cmH2O above ambient air pressure, the plenum chamber having at least one plenum chamber inlet port sized and structured to receive the flow of air at the therapeutic pressure for breathing by the patient;
at least one pair of headgear connector portions configured to connect to a positioning and stabilising structure;
a seal-forming structure partially forming the plenum chamber, the seal-forming structure being constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
wherein the seal forming structure comprises an undercushion and a membrane portion connected to the undercushion;
wherein the undercushion is structured to bend in one or more locations when the patient dons the patient interface.

In examples:
the undercushion is structured to bend in the manner of a first portion of a cross section through the undercushion bending with respect to a second portion of the cross section through the undercushion;
the first portion of the cross section through the undercushion forms a patient-facing side of the undercushion and urges the membrane portion of the seal-forming structure against the patient's face in use, and the second portion of the cross section through the undercushion forms a non-patient facing side of the undercushion in use;
the undercushion is structured to bend proximate the user's nose when the patient dons the patient interface;
the undercushion is structured to bend proximate the user's mouth when the patient dons the patient interface;
the undercushion is structured to bend proximate the user's lip inferior when the patient dons the patient interface;
the undercushion is structured to bend proximate the user's cheeks when the patient dons the patient interface;
the membrane portion is configured to form a seal to at least a pronasale region and the nasal alae of the patient's nose and the seal forming structure is further configured to form a seal around the patient's mouth;
   wherein the undercushion comprises a nasal portion configured to be positioned proximate an inferior periphery of the patient's nose in use, the undercushion forming a nasal recess in the nasal portion configured to at least partially surround the inferior periphery of the patient's nose in use, the membrane portion being supported by the undercushion at a periphery of the nasal recess;
wherein the nasal portion of the undercushion comprises a pair of lateral peripheral portions partially defining the periphery of the nasal recess and located on respective lateral sides of the patient's nose in use, the lateral peripheral portions structured to support the membrane portion superiorly of the patient's nasal alae on respective lateral sides of the patient's nose;
the lateral peripheral portions are flexible and structured to deform medially towards the patient's nose in use;
the lateral peripheral portions are structured to bend medially towards the patient's nose in use;
the undercushion comprises one or more hinge regions at which the undercushion is structured to bend when the patient dons the patient interface;
each of the one or more hinge regions is formed by a channel formed in the undercushion forming a region of reduced thickness;
an oral channel forming one of the one or more hinge regions is provided in a lip inferior region of the undercushion;
the oral channel is provided in cheek regions of the undercushion;
the oral channel extends along a first cheek region, along the lip inferior region and along a second cheek region;
a nasal channel is provided in the nasal recess;
the nasal channel extends laterally around the nasal recess;
the undercushion comprises a pair of posterior support portions each positioned on a respective lateral side of the nasal recess and configured to engage the patient's face medially of and proximate to the nasolabial sulci of the patient's face, and wherein the nasal channel is provided proximate the posterior support portions; and/or
the nasal channel and oral channel meet at respective lateral sides of the undercushion to form a single contiguous channel.

In further examples:
the nasal recess is configured to avoid engaging both lateral sides of the patient's nose simultaneously;
the nasal recess comprises a shape, in a plane parallel to the Frankfort horizontal plane of the patient's head, corresponding to the inferior periphery of the patient's nose;
the nasal recess surrounds substantially all laterally- and anteriorly-facing portions of the inferior periphery of the patient's nose in use;
the nasal recess comprises an inwardly-facing wall at least partially surrounding the inferior periphery of the patient's nose in use;
the inwardly-facing wall faces partially inwardly and partially superiorly;
the inwardly-facing wall extends from at or proximate one of the patient's cheeks around the patient's nose to at or proximate the other of the patient's cheeks; and/or
the membrane portion is attached to the undercushion around the periphery of the nasal recess.

In further examples:
the patient interface comprises a fascia portion partially forming the plenum chamber, the undercushion being attached to the fascia portion;
the fascia portion is formed from foam covered with a textile material on a non-patient-facing surface of the fascia portion;
the fascia portion comprises a textile layer on the patient-facing surface;
the undercushion is formed from foam;
the undercushion comprises a skinned surface;
the undercushion is formed from a polyurethane, for example a thermoplastic polyurethane;
the fascia portion comprises a curved three-dimensional shape in use;
the fascia portion is thermoformed into the curved three-dimensional shape so as to support itself in the curved three-dimensional shape;
the undercushion is formed from compression cut foam;
the undercushion is formed from moulded foam;
the fascia portion is unable to support itself in the curved three-dimensional shape;
the undercushion is formed from moulded foam and supports the fascia portion in the curved three-dimensional shape;
the undercushion is configured to hold the membrane portion substantially taut when the patient interface is not in use;
the membrane portion is bonded to the undercushion and/or the fascia portion;
the membrane portion comprises a first hole through which air can flow to both the patient's nares, in use;
the membrane portion comprises a second hole through which air can flow to the patient's mouth, in use; and/or
the membrane portion is at least partially formed from a textile material.

Another aspect is a patient interface comprising a cushion module according to any of the above aspects or examples, wherein the patient interface comprises a positioning and stabilising structure configured to apply a force to the cushion module to hold the cushion module in sealing position in use. The positioning and stabilising structure may comprise a pair of first strap portions configured to connect to the cushion module. In some examples the positioning and stabilising structure may comprise a pair of superior strap portions and a pair of inferior strap portions configured to connect to the cushion module.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

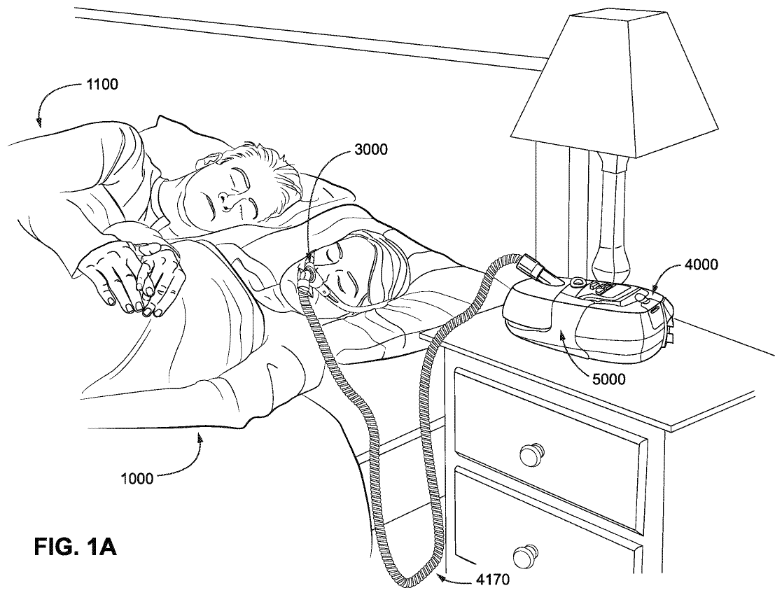

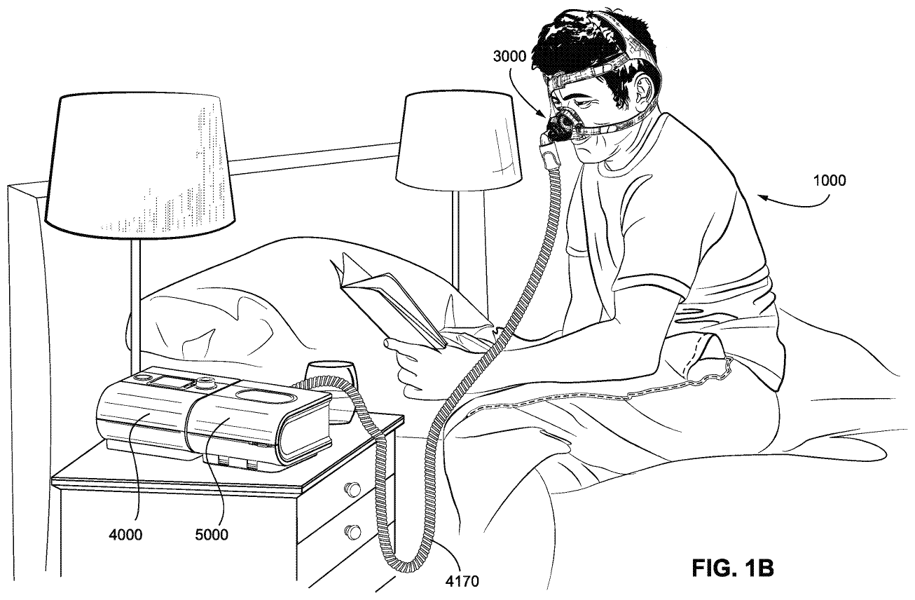

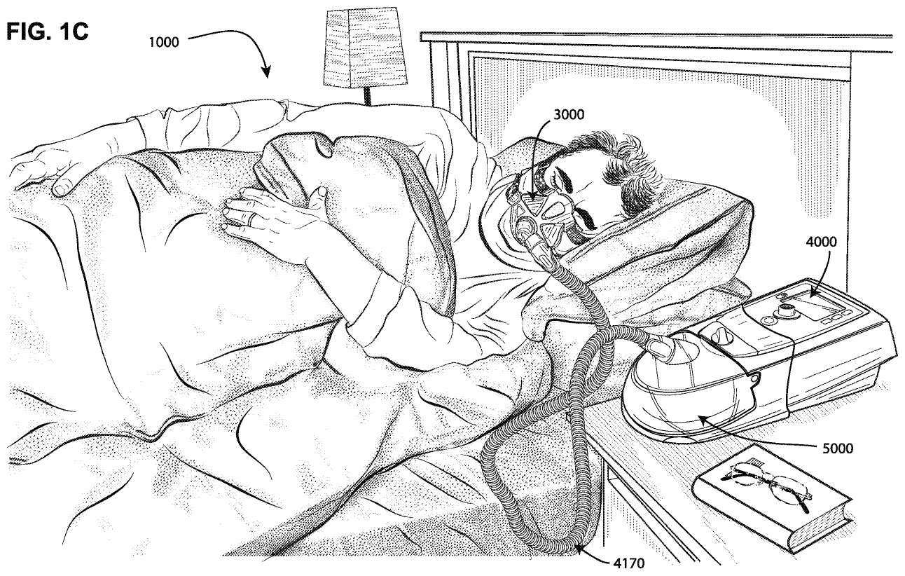

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

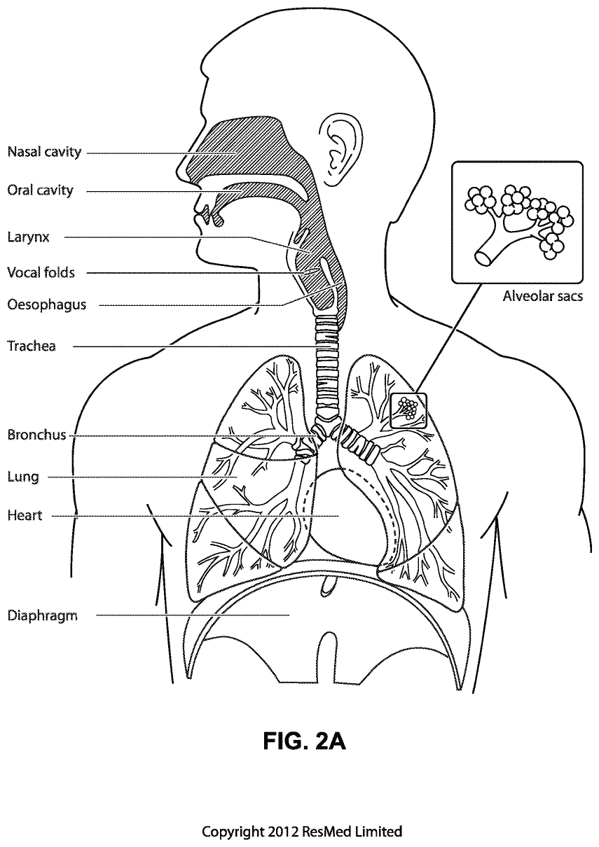

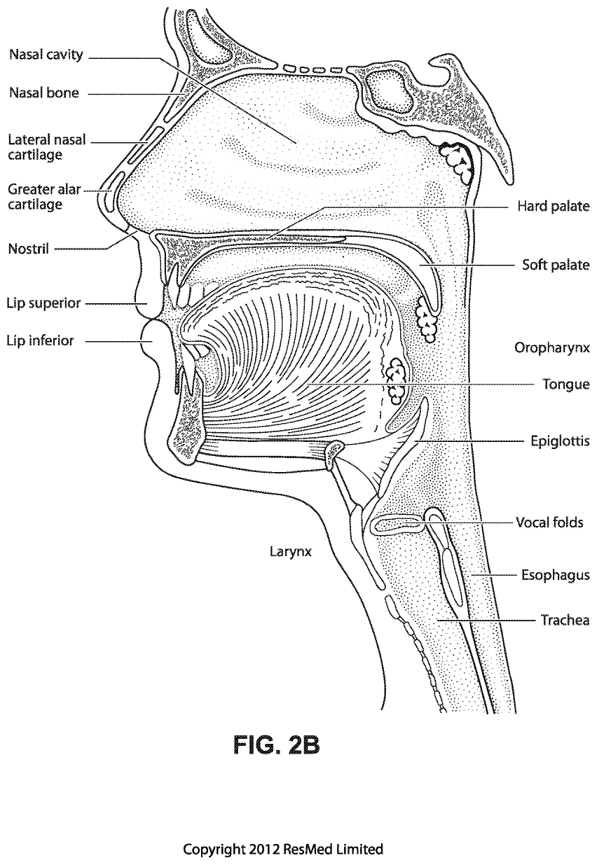

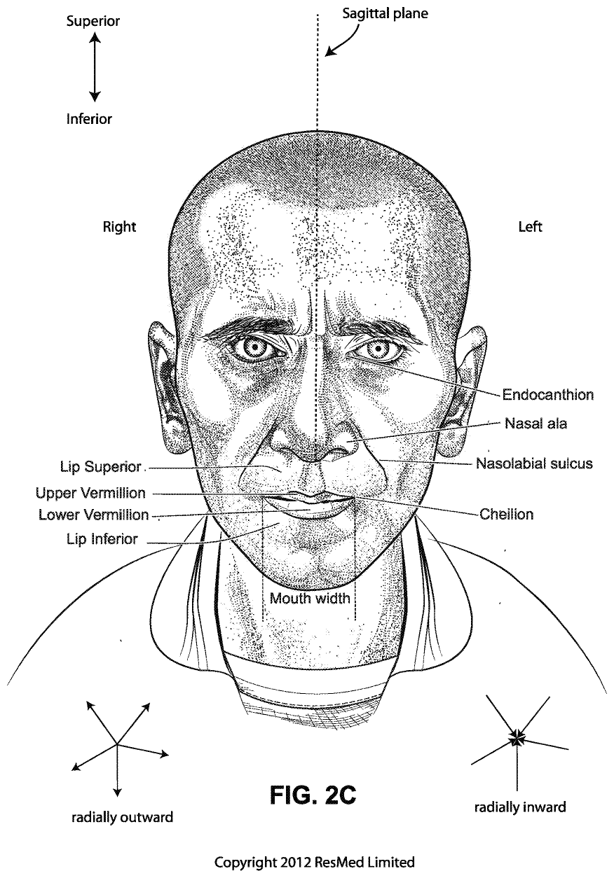

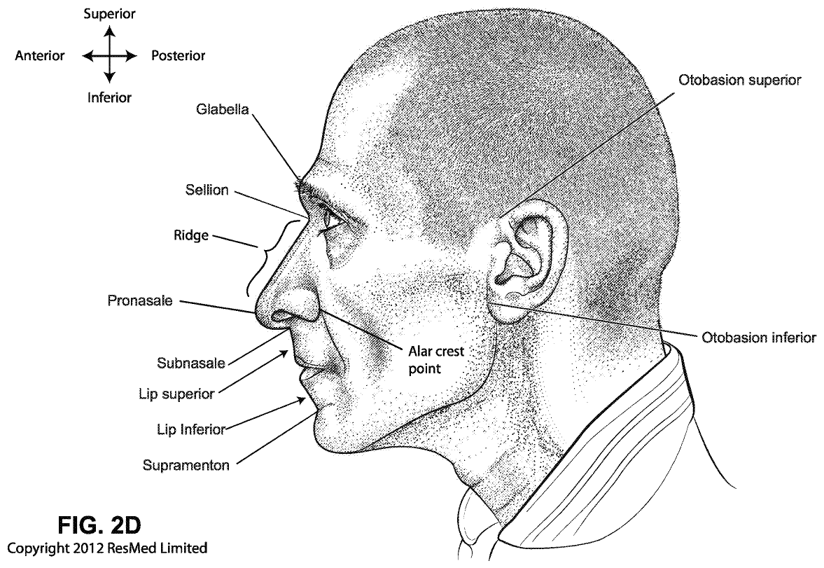

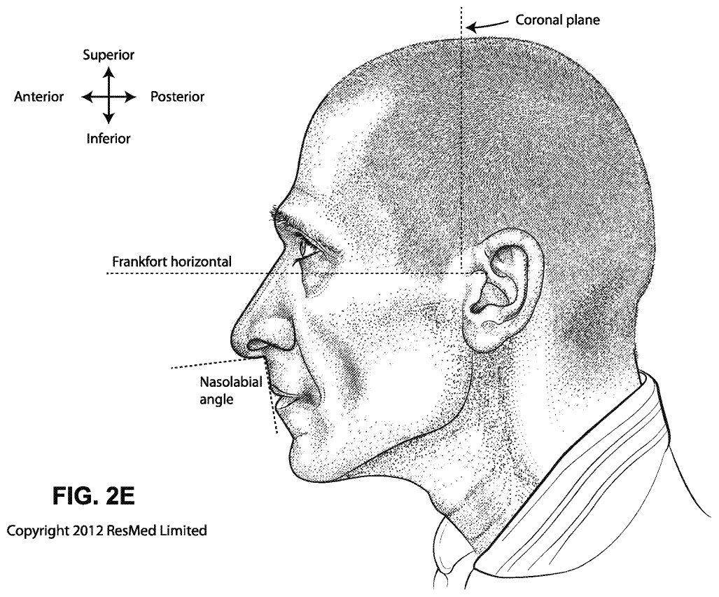

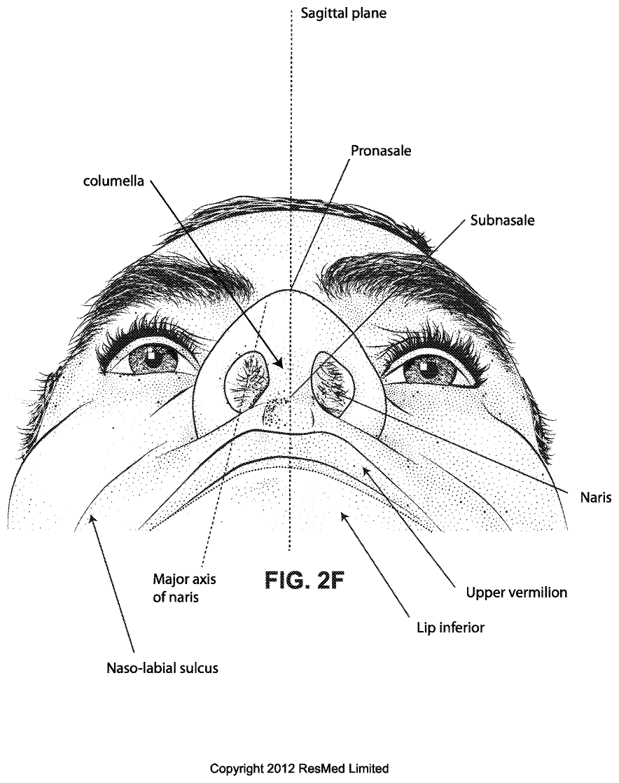

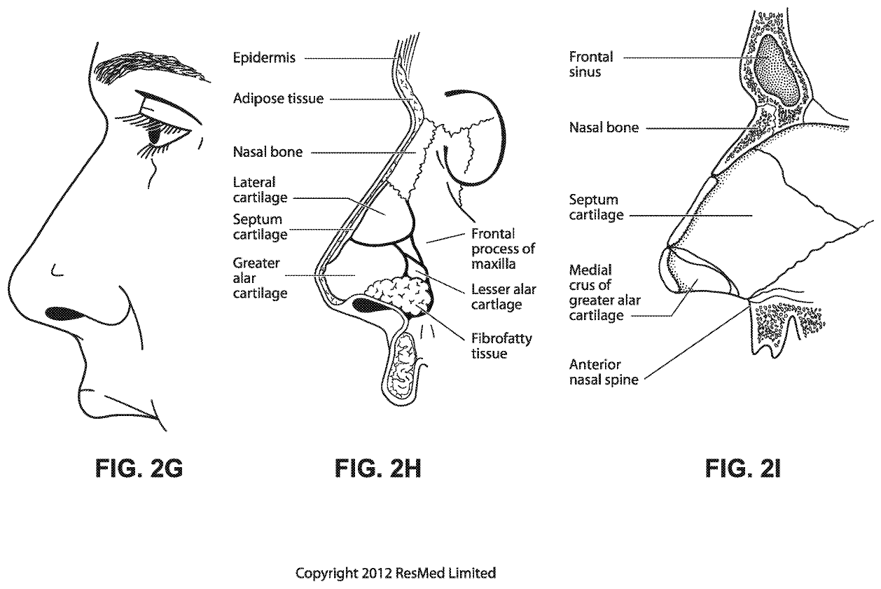

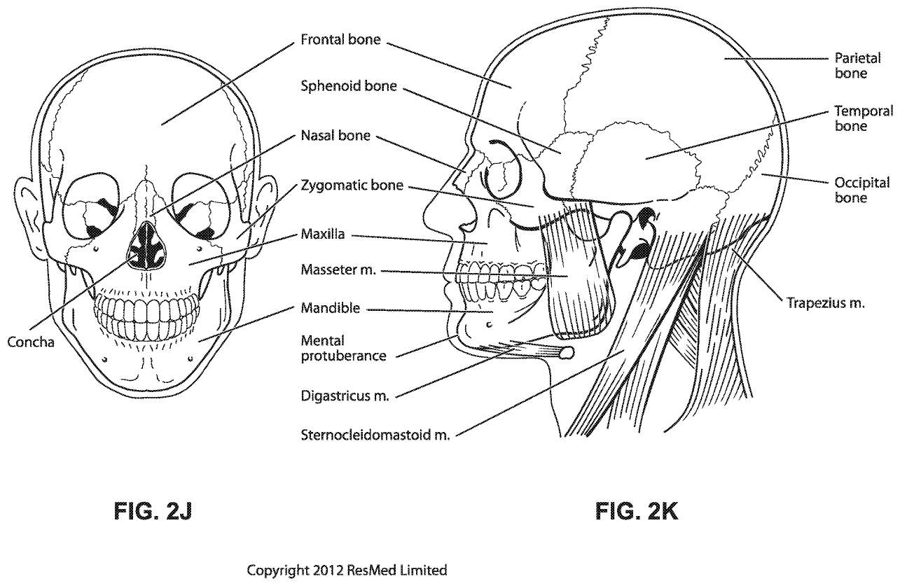

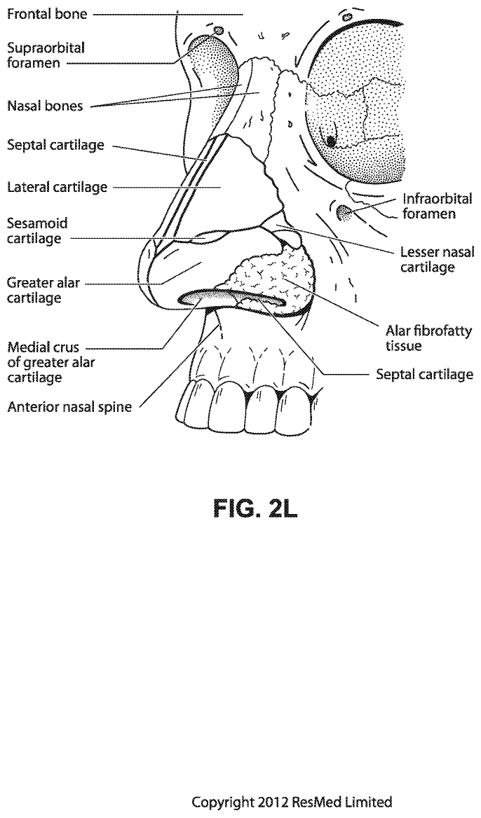

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 4.3 Patient Interface

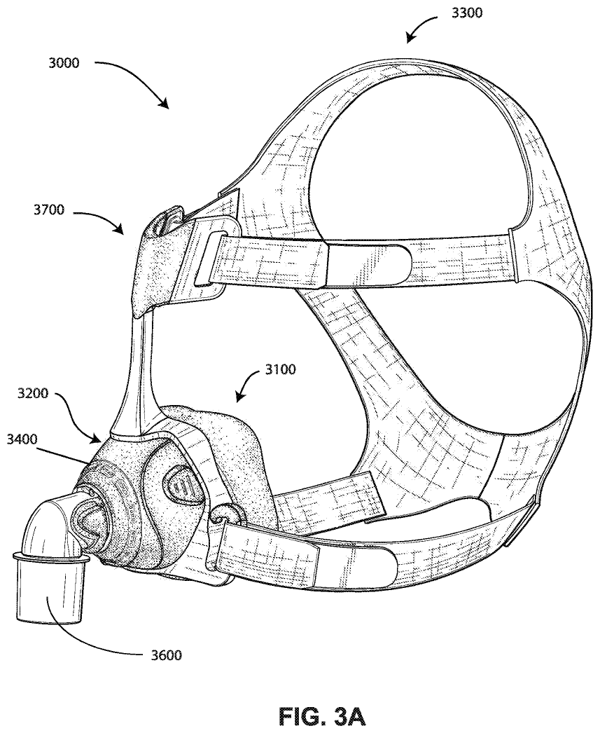

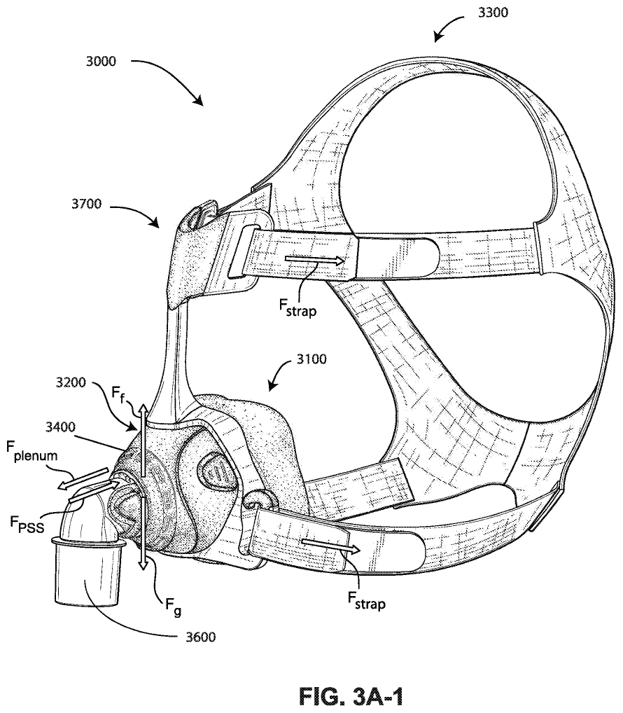

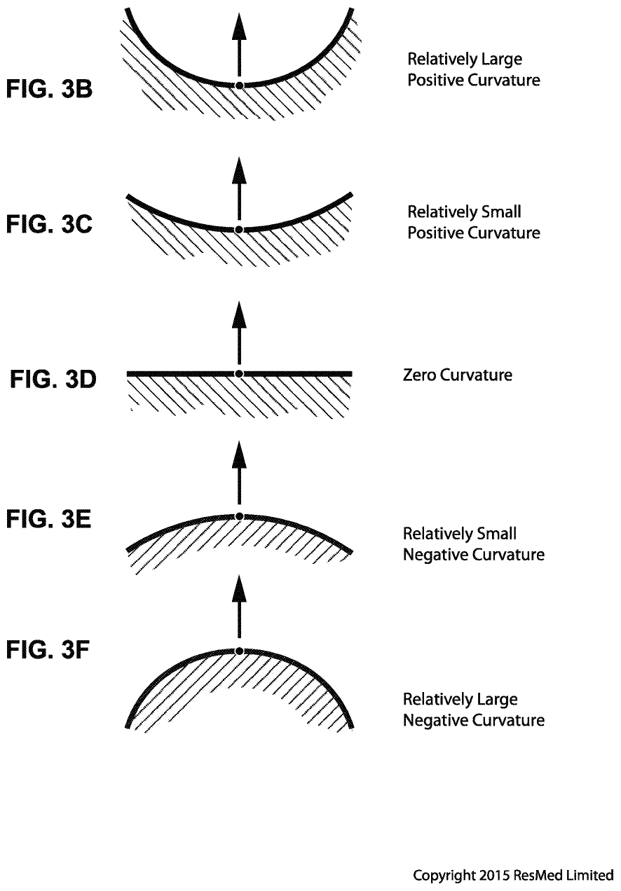

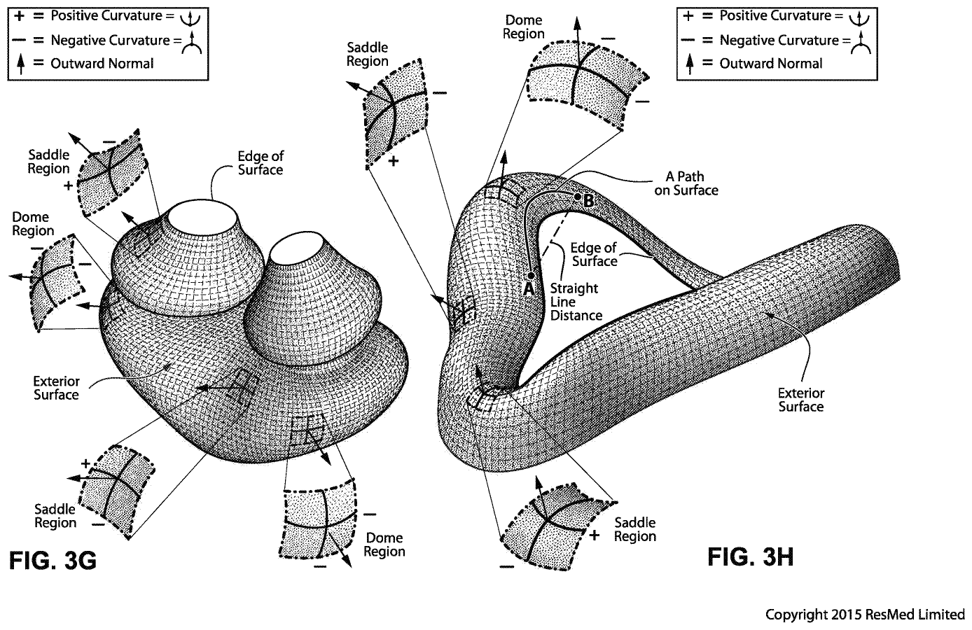

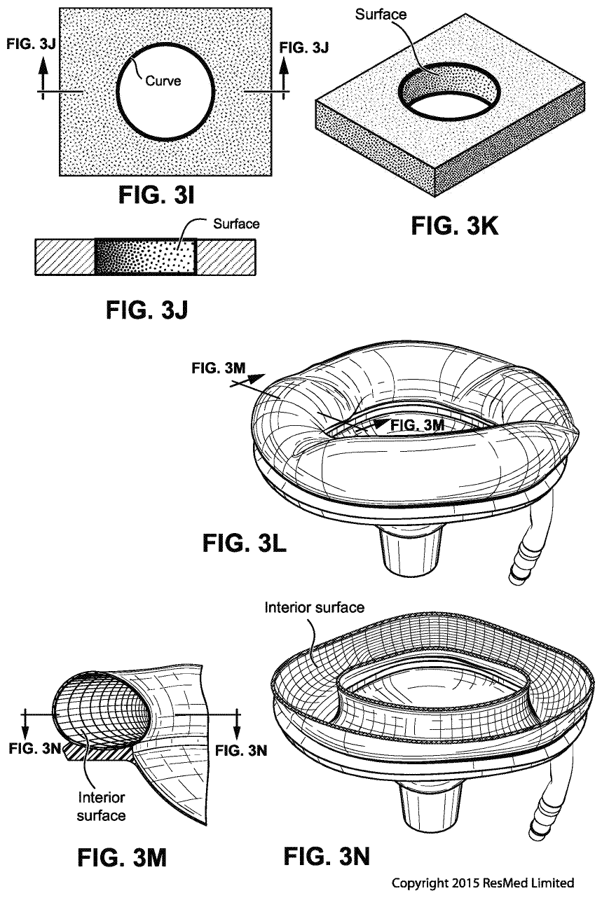

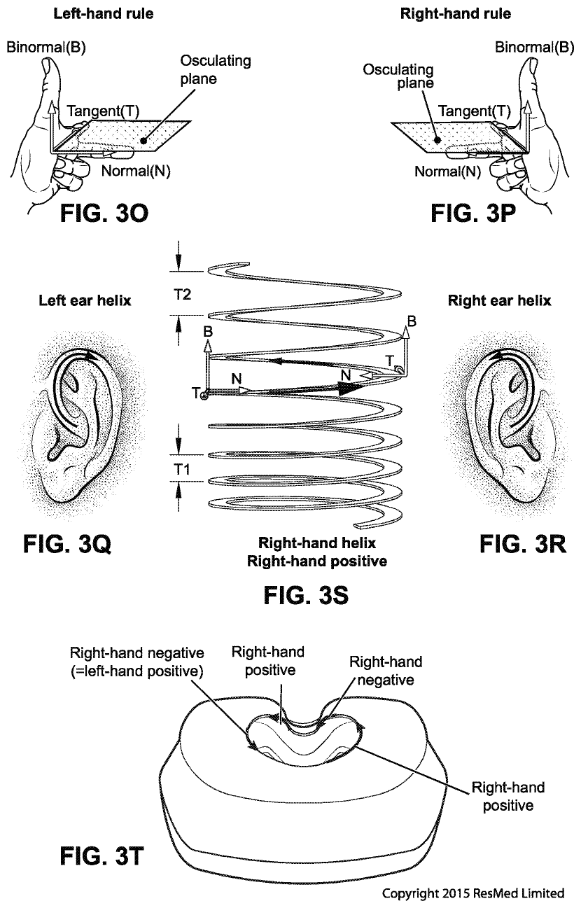

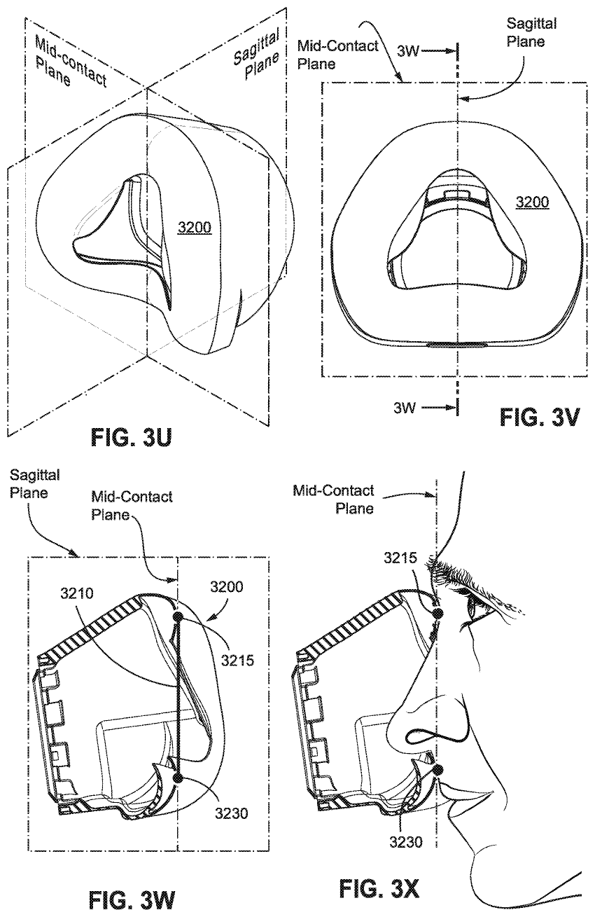

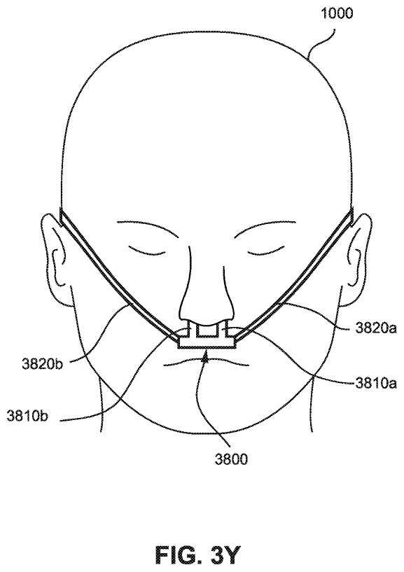

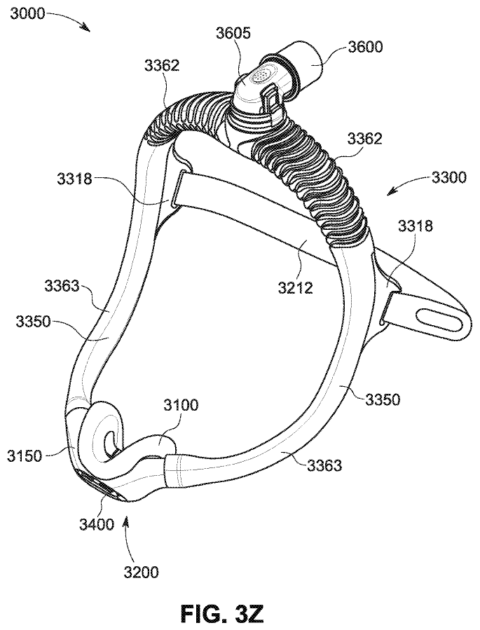

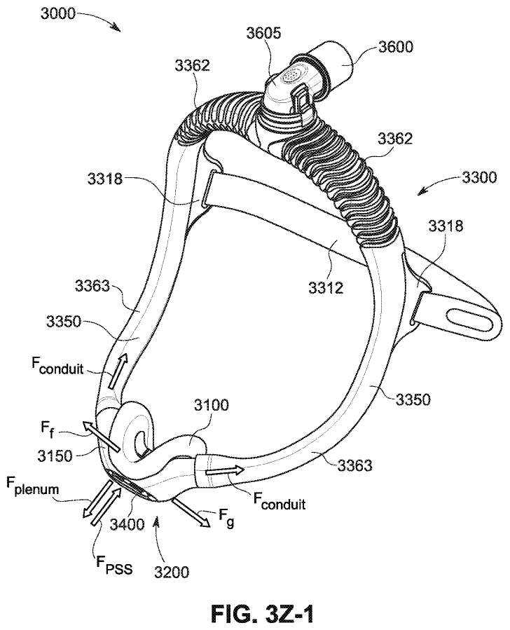

FIG. 3A shows a patient interface in the form of a nasal mask in accordance with one form of the present technology.

FIG. 3A-1 shows forces acting on the patient interface of FIG. 3A, while in use.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3215 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3215 sits approximately on the sellion, while the inferior point 3230 sits on the lip superior.

FIG. 3Y shows a patient interface in the form of a nasal cannula in accordance with one form of the present technology.

FIG. 3Z shows a patient interface having conduit headgear, in accordance with one form of the present technology.

FIG. 3Z-1 shows forces acting on the patient interface of FIG. 3Z, while in use.

### 4.4 RPT Device

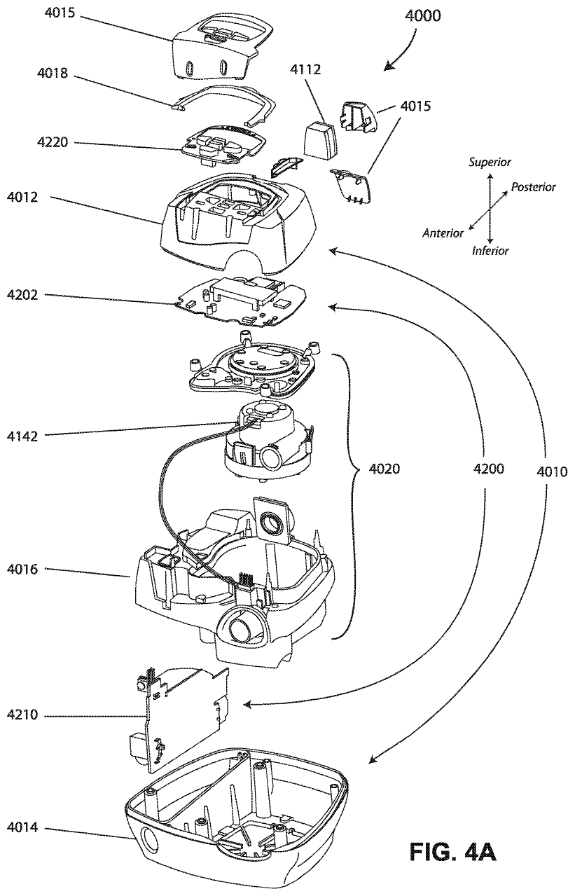

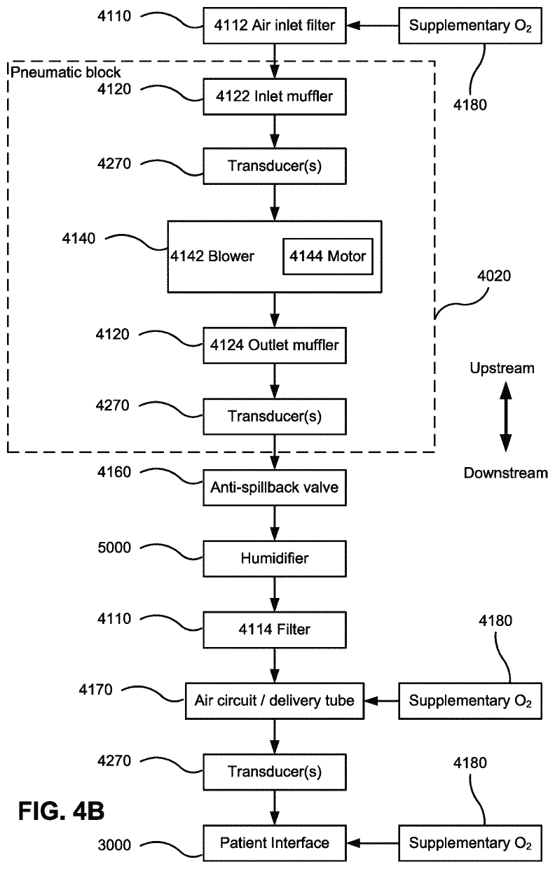

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 4.5 Humidifier

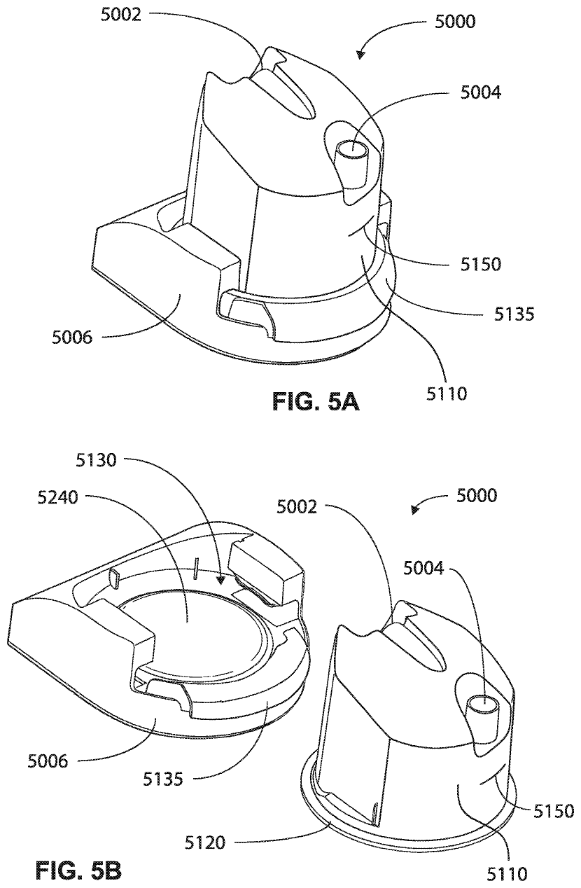

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

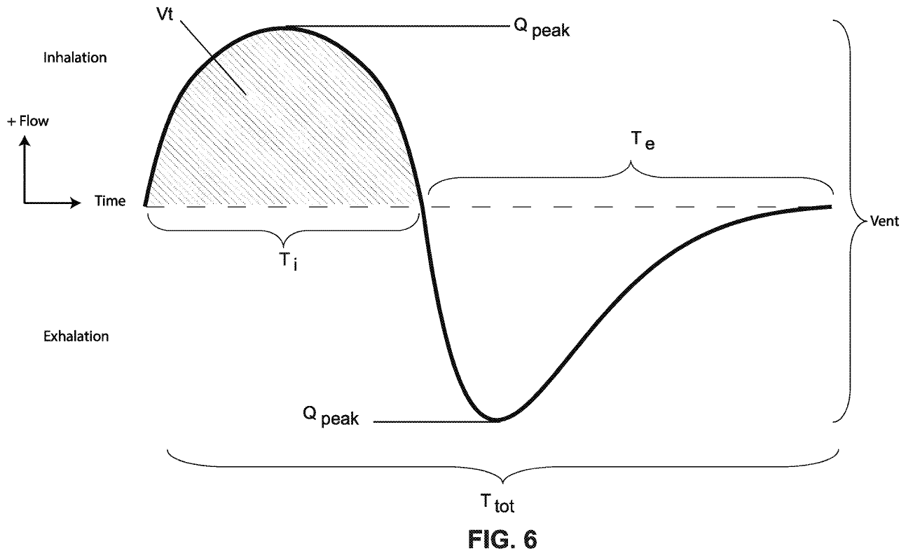

FIG. 6 shows a model typical breath waveform of a person while sleeping.

### 4.7 Modularity

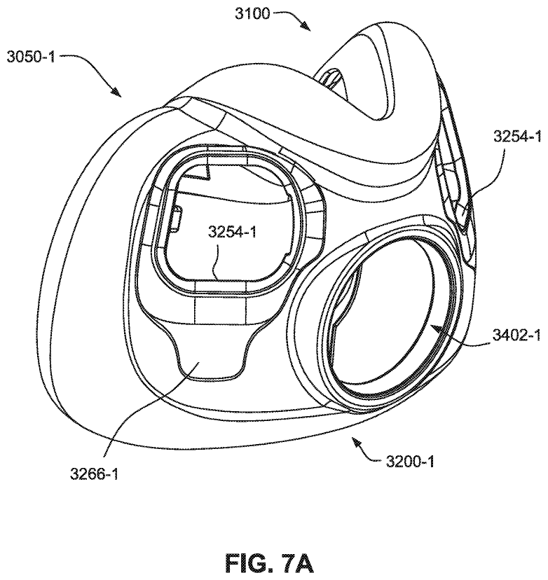

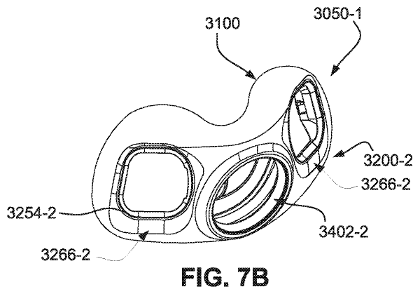

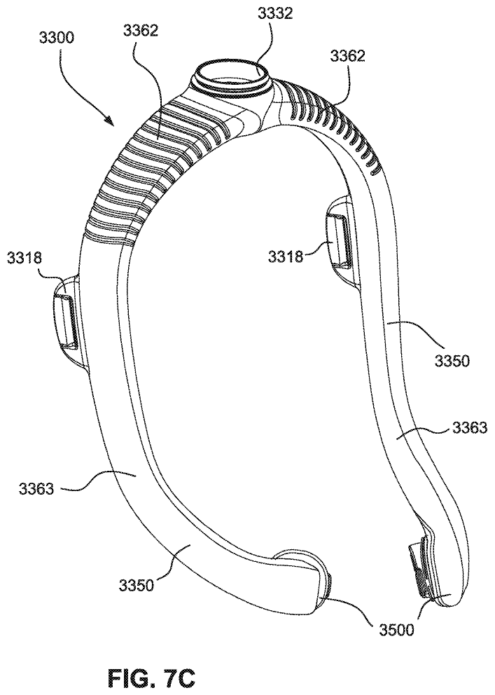

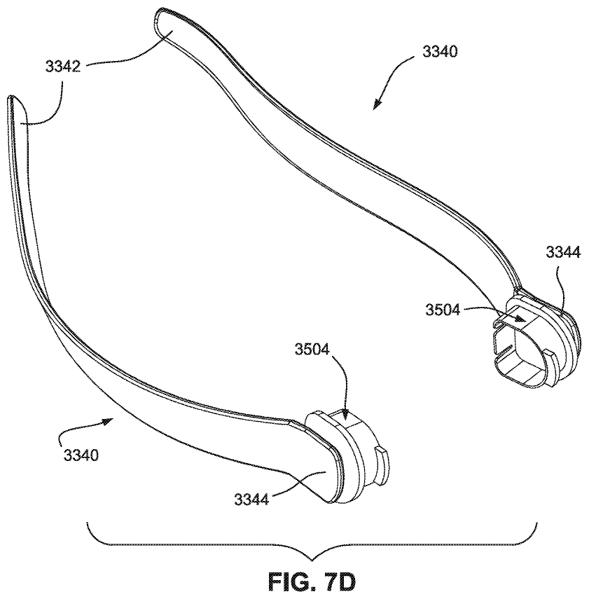

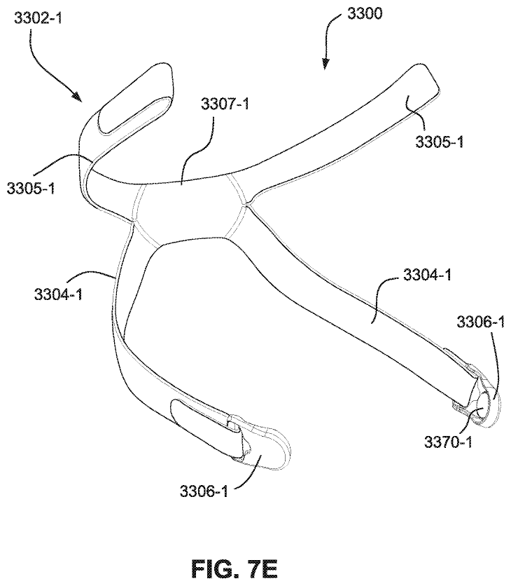

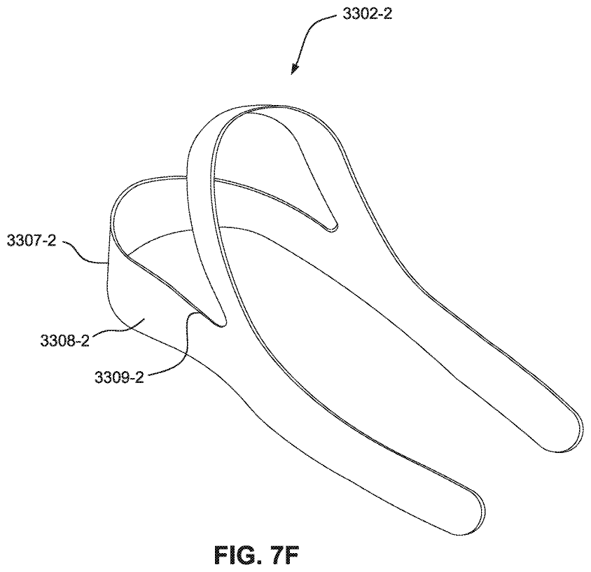

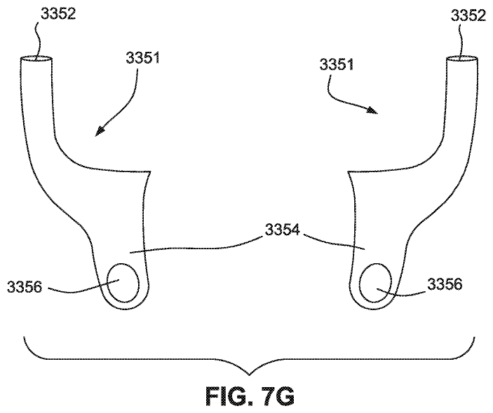

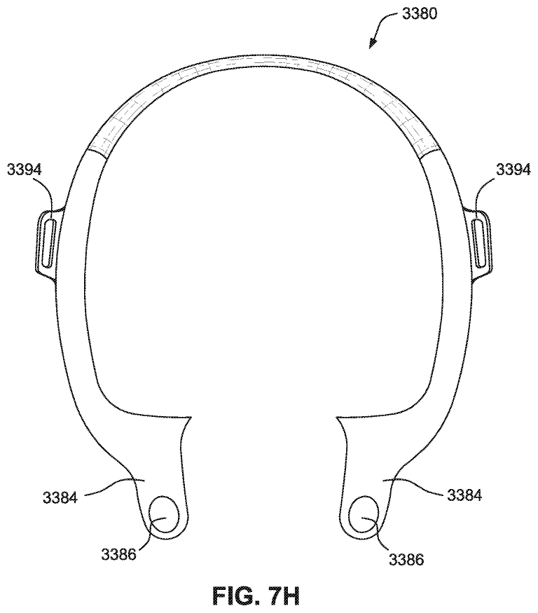

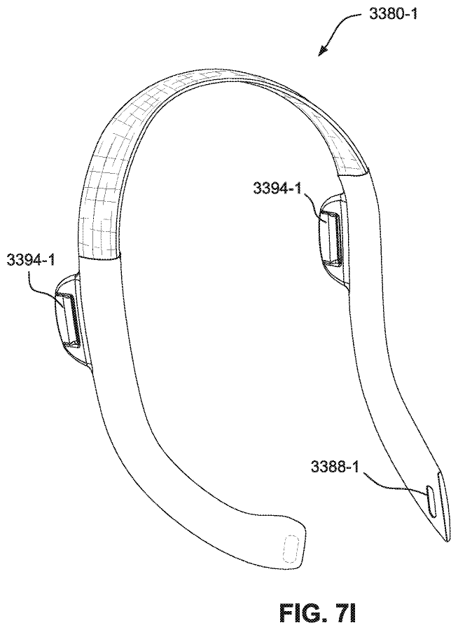

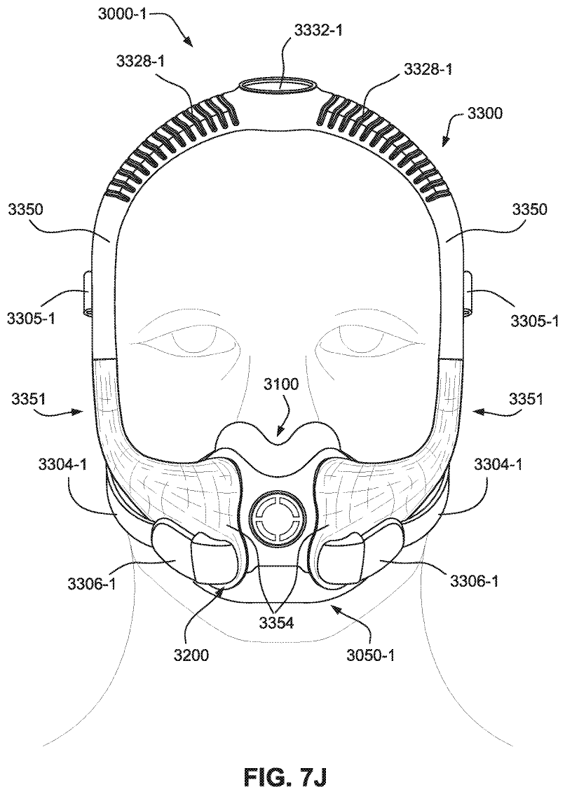

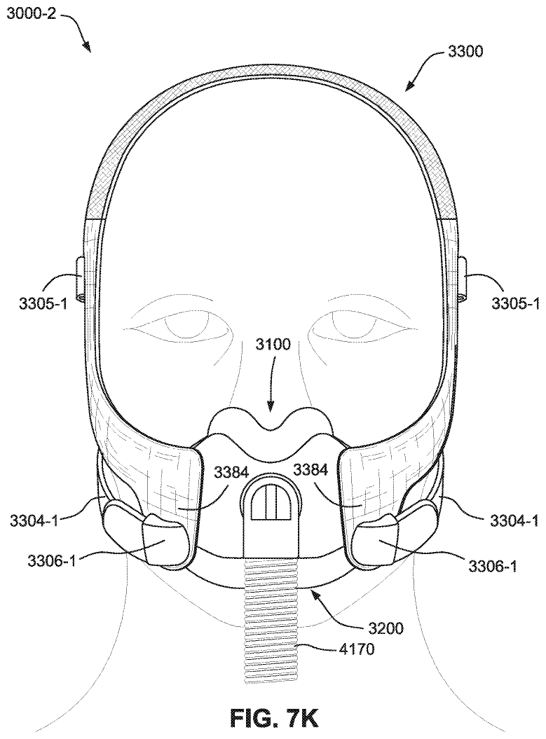

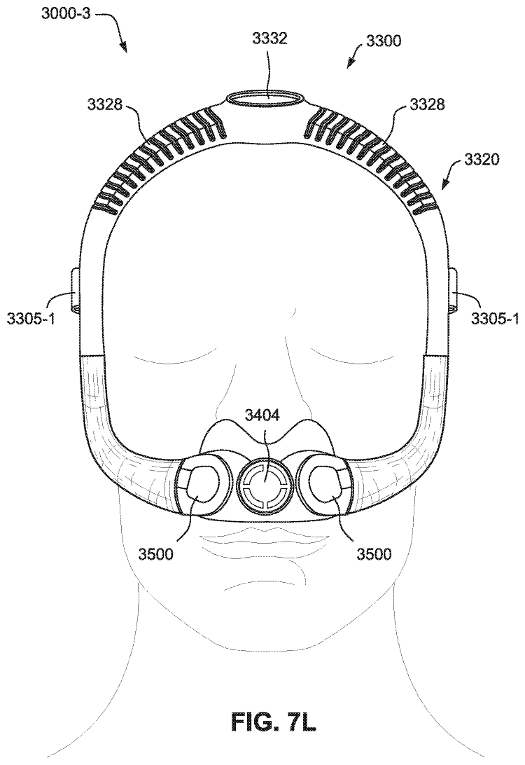

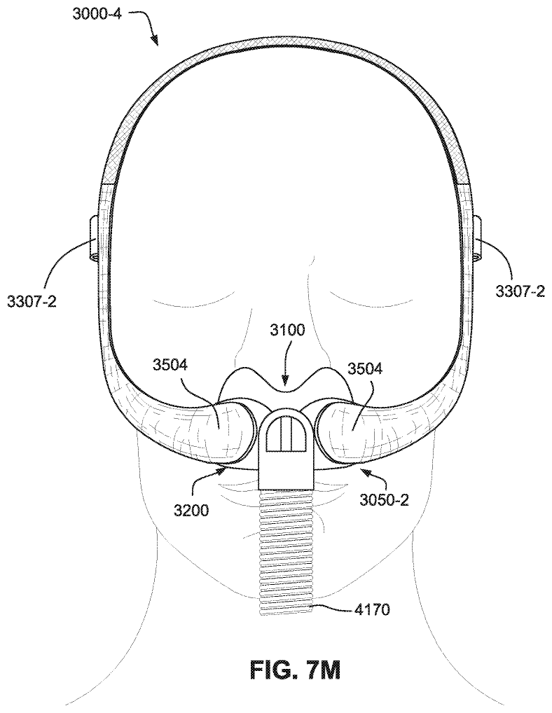

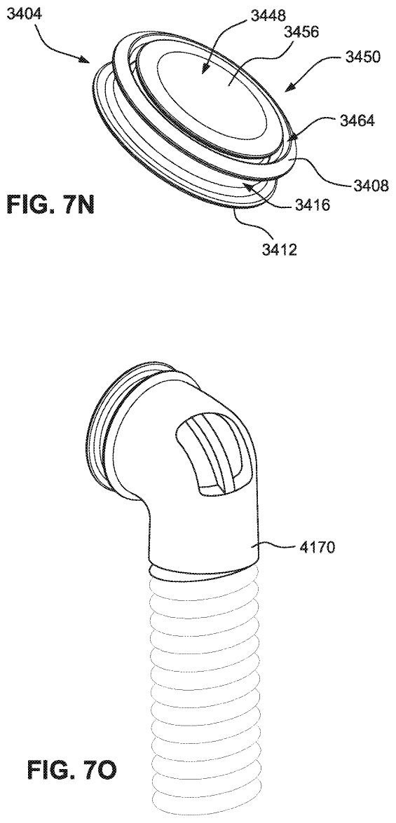

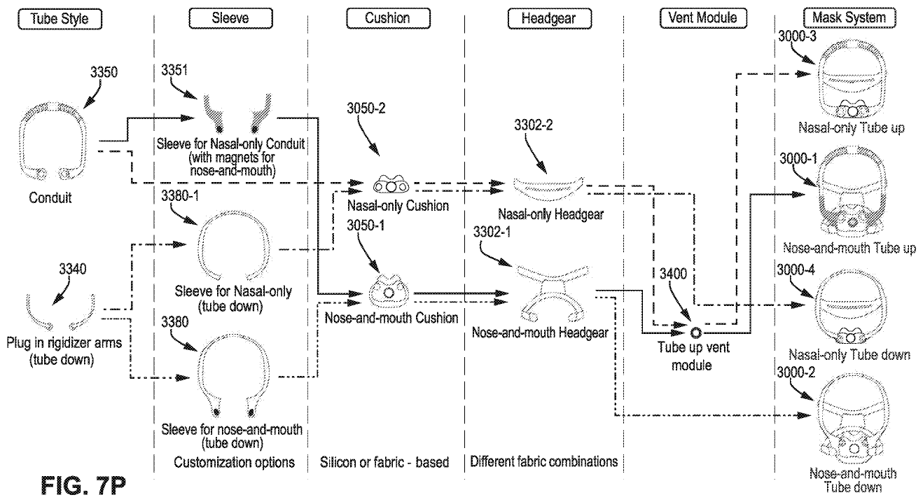

FIG. 7A shows a perspective view of a cushion of a patient interface configured to be worn by a patient and convey pressurized air to the patient's nose and the patient's mouth.

FIG. 7B shows a perspective view of a cushion of a patient interface configured to be worn by a patient and convey pressurized air to the patient's nose.

FIG. 7C shows a perspective view of tubes usable with either the cushion of FIG. 7A or the cushion of FIG. 7B.

FIG. 7D shows a perspective view of rigidiser arms usable with either the cushion of FIG. 7A of the cushion of FIG. 7B.

FIG. 7E shows a perspective view of headgear straps usable with the cushion of FIG. 7A.

FIG. 7F shows a perspective view of headgear straps usable with the cushion of FIG. 7B.

FIG. 7G shows a front view of a pair of sleeves that is removably fitted to either the tubes of FIG. 7C or the rigidiser arms of FIG. 7D.

FIG. 7H shows a front view of a full sleeve that is removably fitted to the rigidiser arms of FIG. 7D.

FIG. 7I shows a front perspective view of yet another alternate form of a full sleeve that is removably fitted to the rigidiser arms of FIG. 7D.

FIG. 7J is a front view of a patient wearing the cushion of FIG. 7A connected to the tubes of FIG. 7C, the headgear straps of FIG. 7E, and the sleeves of FIG. 7G.

FIG. 7K is a front view of a patient wearing the cushion of FIG. 7A connected to the rigidiser arms of FIG. 7D, the headgear straps of FIG. 7E, and the sleeve of FIG. 7H.

FIG. 7L is a front view of a patient wearing the cushion of FIG. 7B connected to the conduit headgear of FIG. 7C, and the headgear straps of FIG. 7F.

FIG. 7M is a front view of a patient wearing the cushion of FIG. 7B connected to the rigidisier arms of FIG. 7D, the headgear straps of FIG. 7F, and the sleeve of FIG. 7I.

FIG. 7N is an isolated perspective view of the vent of FIG. 7L.

FIG. 7O is an isolated perspective view of a portion of the air circuit of FIG. 7M.

FIG. 7P is a schematic view illustrating the possible combinations of the patient interfaces.

### 4.8 Further Examples of the Present Technology

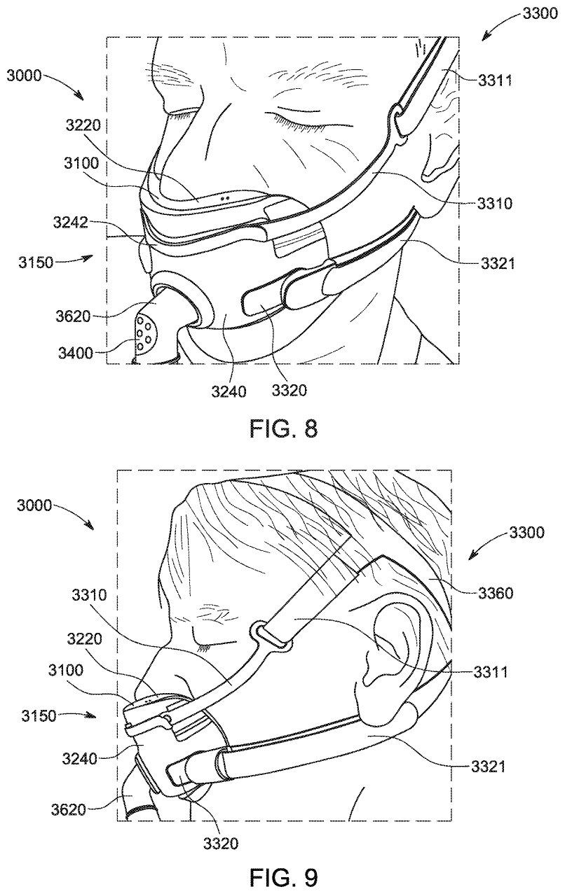

FIG. 8 is a perspective view illustration of a patient interface according to one example of the present technology in use by a patient.

FIG. 9 is a lateral view illustration of the patient interface shown in FIG. 8 in use by a patient.

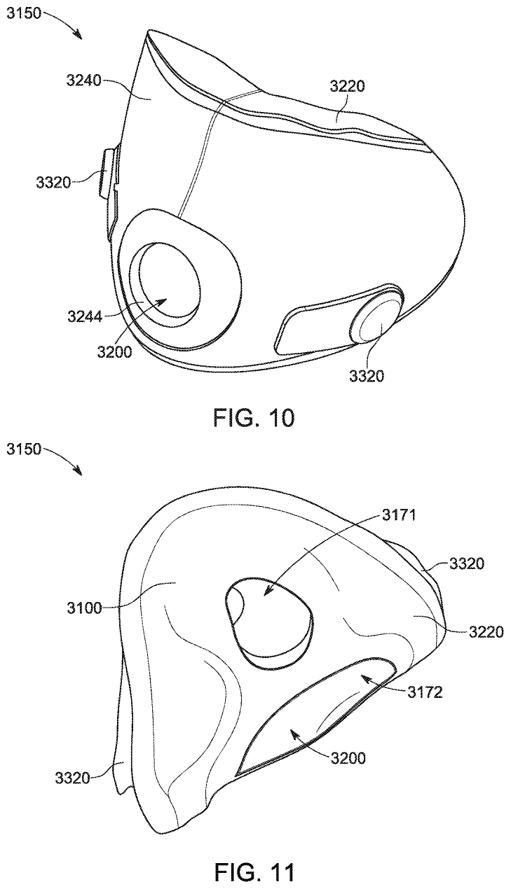

FIG. 10 is an anterior perspective view illustration of a cushion module of the patient interface of FIG. 8.

FIG. 11 is a posterior perspective view illustration of the cushion module of FIG. 10.

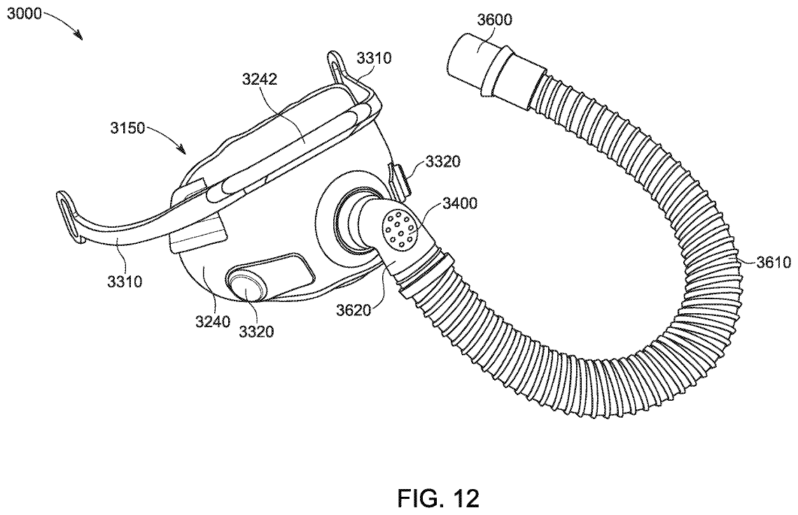

FIG. 12 is a perspective view illustration of the patient interface shown in FIG. 8.

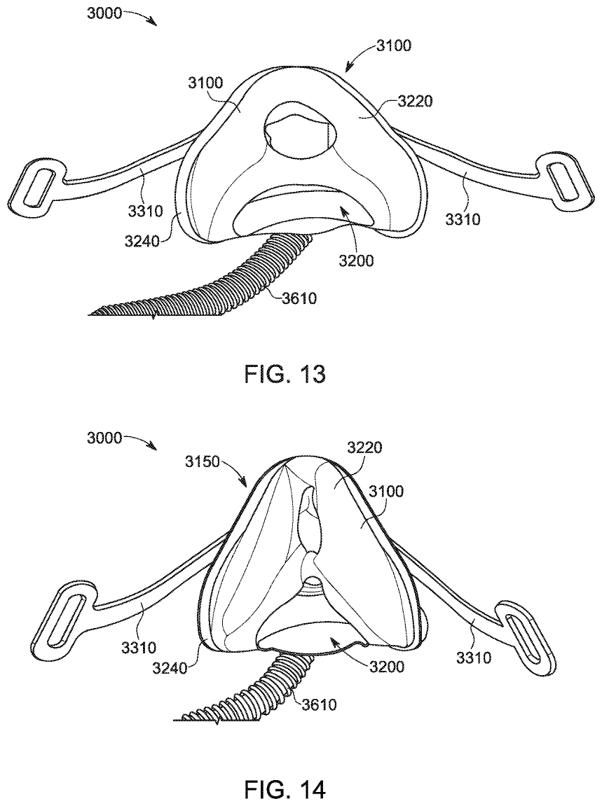

FIG. 13 is a posterior perspective view illustration of the patient interface shown in FIG. 8 in a wide configuration.

FIG. 14 is a posterior perspective view illustration of the patient interface shown in FIG. 8 in a narrow configuration.

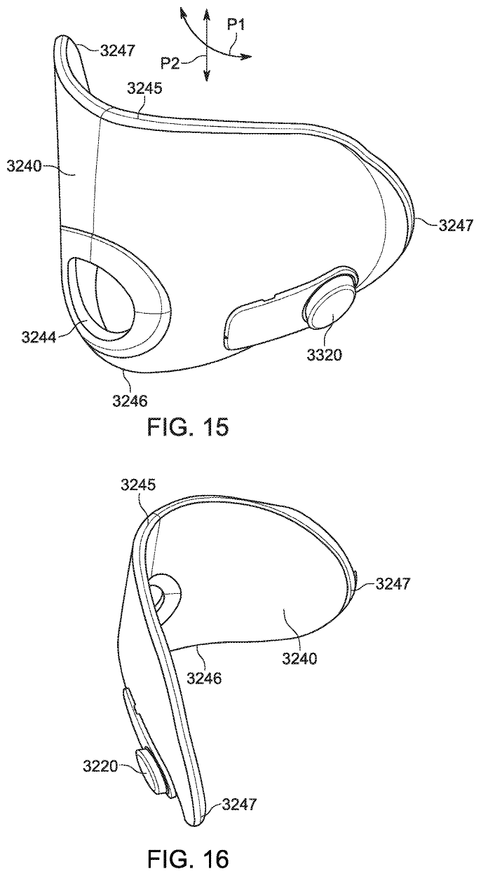

FIG. 15 is an anterior perspective view illustration of a fascia portion of the patient interface shown in FIG. 8.

FIG. 16 is a superior lateral perspective view illustration of the fascia portion shown in FIG. 15.

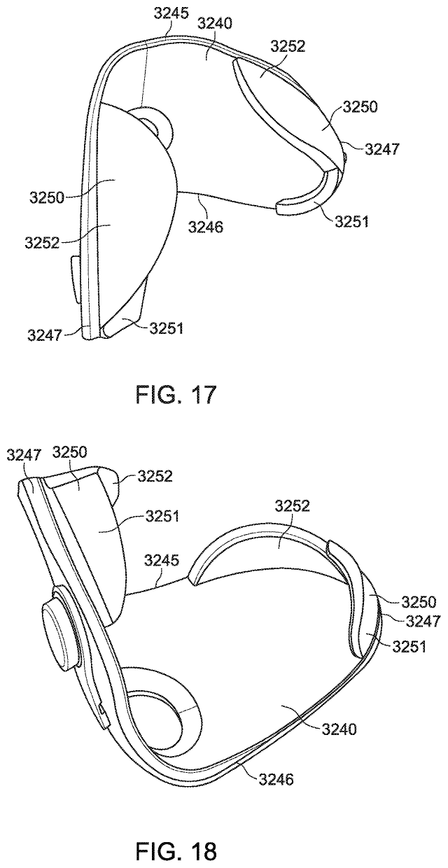

FIG. 17 is a posterior lateral perspective view illustration of a fascia portion of a patient interface according to another example of the present technology.

FIG. 18 is a posteroinferior perspective view illustration of the fascia portion shown in FIG. 17.

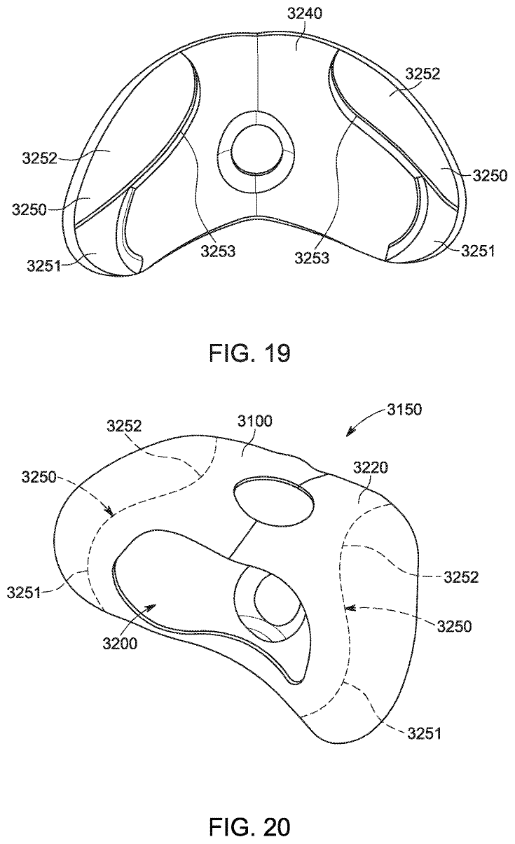

FIG. 19 is a posterior view illustration of the fascia portion shown in FIG. 17.

FIG. 20 is a posterior perspective view illustration of a cushion module including the fascia portion shown in FIG. 17.

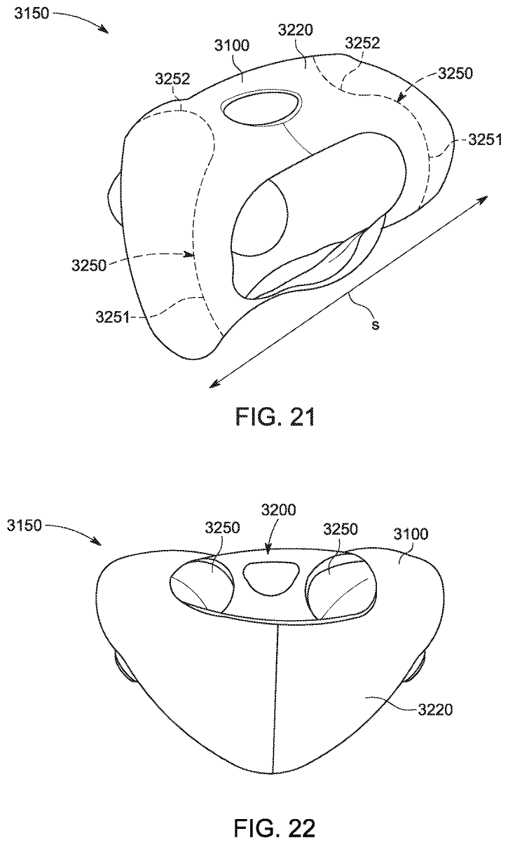

FIG. 21 is another posterior perspective view illustration of the cushion module shown in FIG. 20.

FIG. 22 is an inferior perspective view illustration of the cushion module shown in FIG. 20.

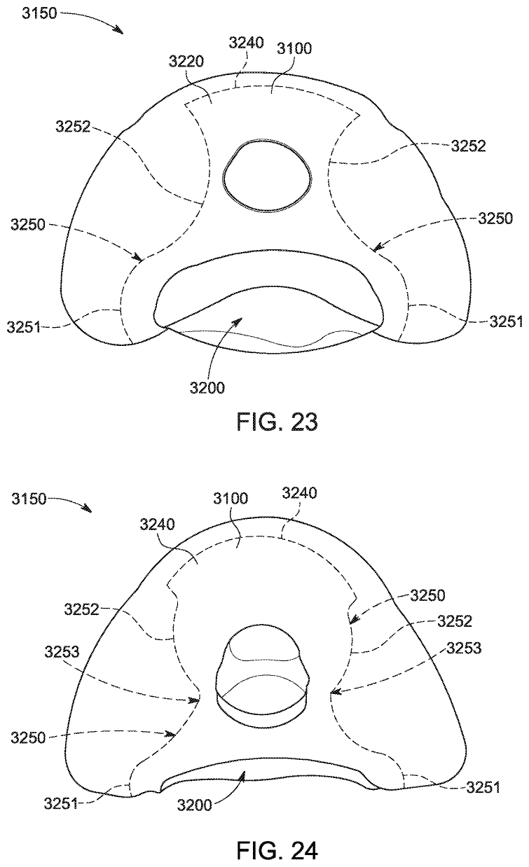

FIG. 23 is a posterosuperior view illustration of the cushion module shown in FIG. 20.

FIG. 24 is a posterosuperior view illustration of a cushion module according to another example of the present technology.

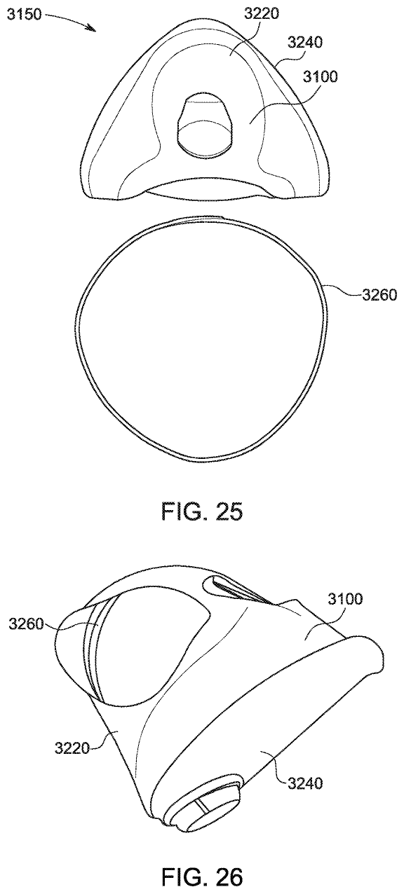

FIG. 25 is a perspective view of a cushion module according to another example of the present technology and a resilient member of the cushion module in an unassembled configuration.

FIG. 26 is a lateral perspective view illustration of the cushion module shown in FIG. 25 in an unassembled configuration with the resilient member visible through an oral hole of the cushion module.

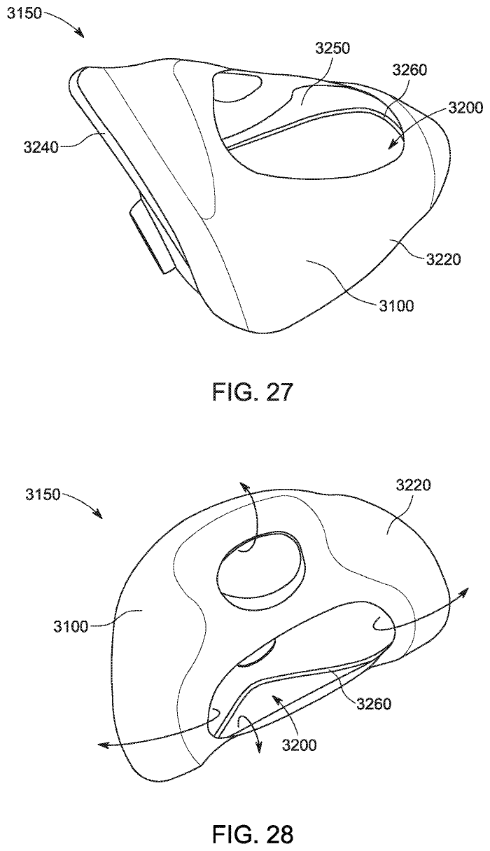

FIG. 27 is a lateral posteroinferior perspective view illustration of the cushion module shown in FIG. 26 with the resilient member visible through an oral hole of the cushion module.

FIG. 28 is a posterosuperior perspective view illustration of the cushion module shown in FIG. 26 with the resilient member visible through an oral hole of the cushion module.

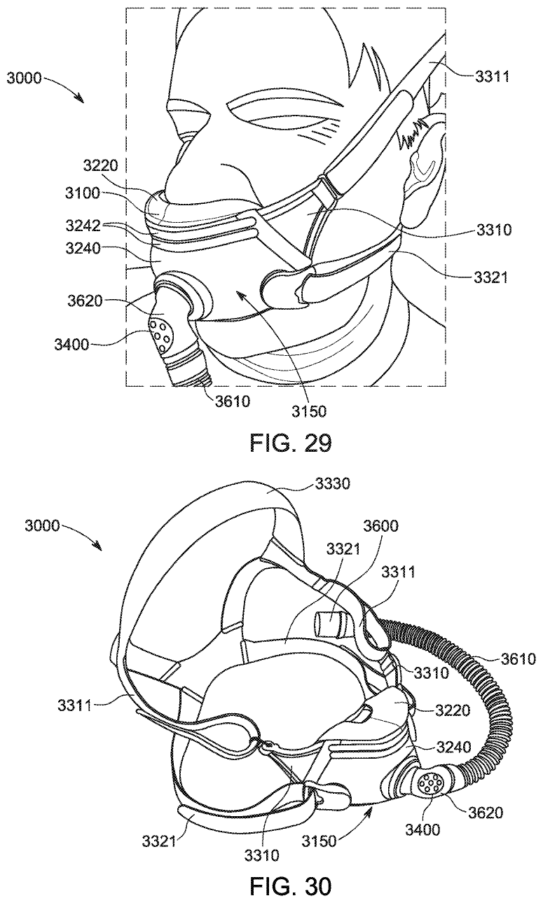

FIG. 29 is a perspective view illustration of a patient interface according to another example of the present technology in use by a patient.

FIG. 30 is a perspective view illustration of the patient interface shown in FIG. 29 in isolation.

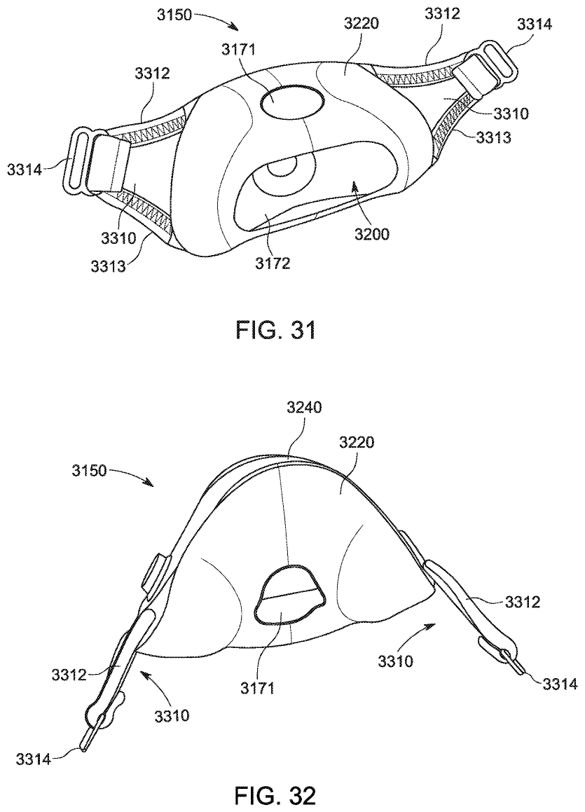

FIG. 31 is a posterior perspective view illustration of a cushion module of the patient interface shown in FIG. 29.

FIG. 32 is a posterosuperior perspective view illustration of the cushion module shown in FIG. 31.

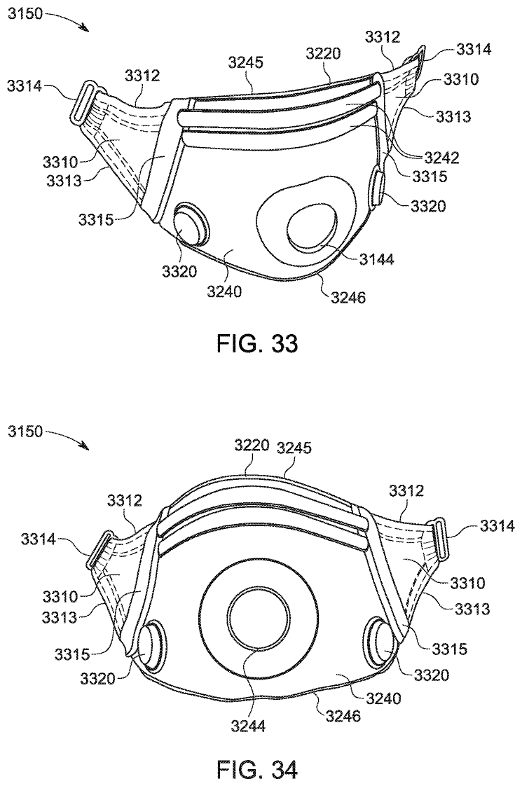

FIG. 33 is an anterior lateral perspective view illustration of the cushion module shown in FIG. 31.

FIG. 34 is an anterior view illustration of the cushion module shown in FIG. 31.

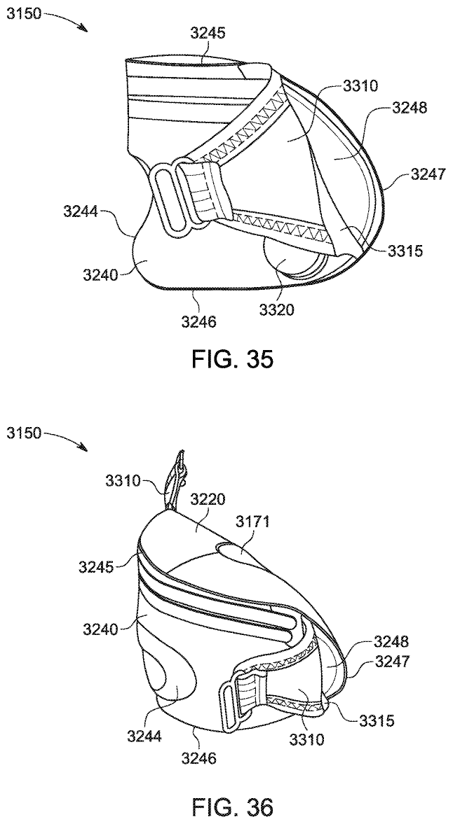

FIG. 35 is a lateral view illustration of the cushion module shown in FIG. 31 with a superior headgear connection portion pulled towards an anterior direction.

FIG. 36 is a superior lateral perspective view illustration of the cushion module shown in FIG. 31 with a superior headgear connection portion pulled towards an anterior direction.

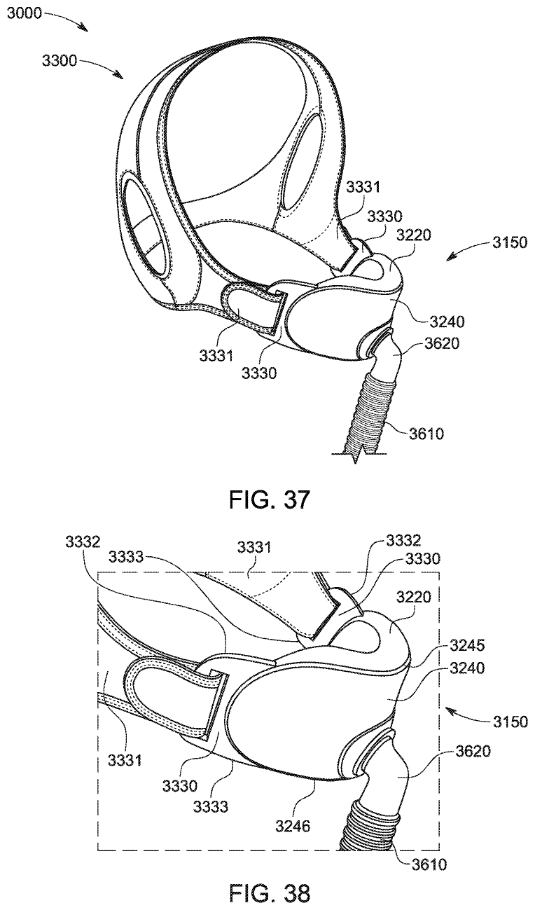

FIG. 37 is a perspective view of a patient interface according to another example of the present technology.

FIG. 38 is a detail perspective view of a cushion module of the patient interface shown in FIG. 37.

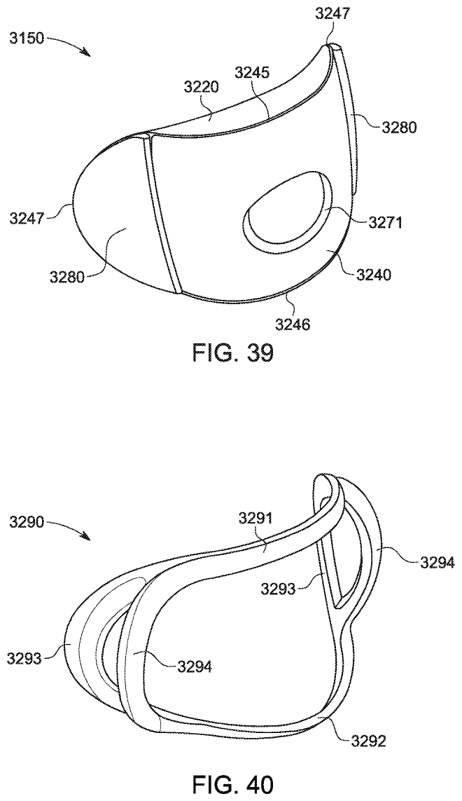

FIG. 39 is a perspective view of the cushion module according to another example of the present technology.

FIG. 40 is a perspective view of a frame according to an example of the present technology, for the cushion module shown in FIG. 39.

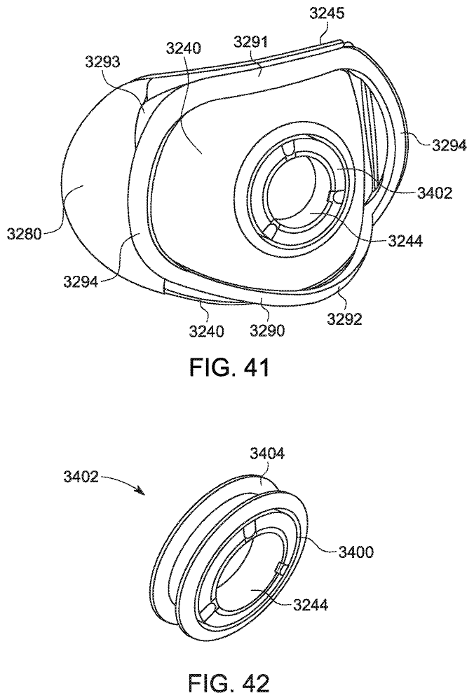

FIG. 41 is a perspective view of the cushion module shown in FIG. 39 assembled with the frame of FIG. 40 and the vent module shown in FIG. 42.

FIG. 42 is a vent module according to an example of the present technology, for the cushion module shown in FIG. 39.

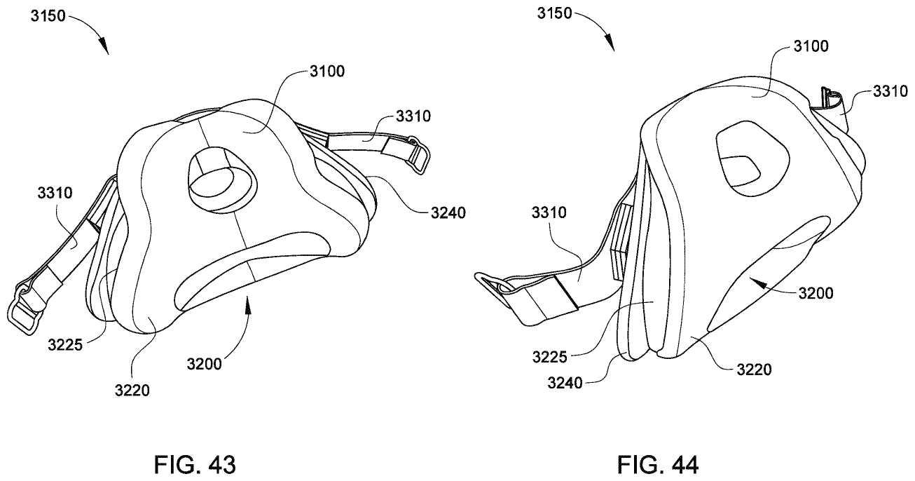

FIGS. 43-48 show illustrations of a patient interface and components thereof according to another example of the present technology.

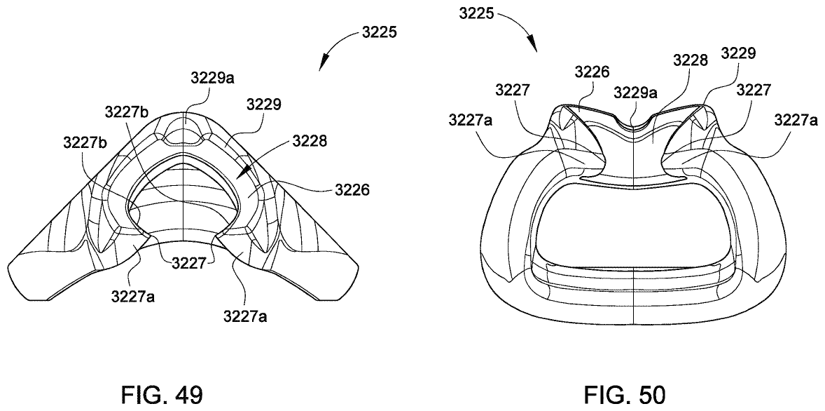

FIG. 49 shows a superior view illustration of an undercushion for a patient interface according to another example of the present technology.

FIG. 50 shows a posterior view illustration of the undercushion shown in FIG. 49.

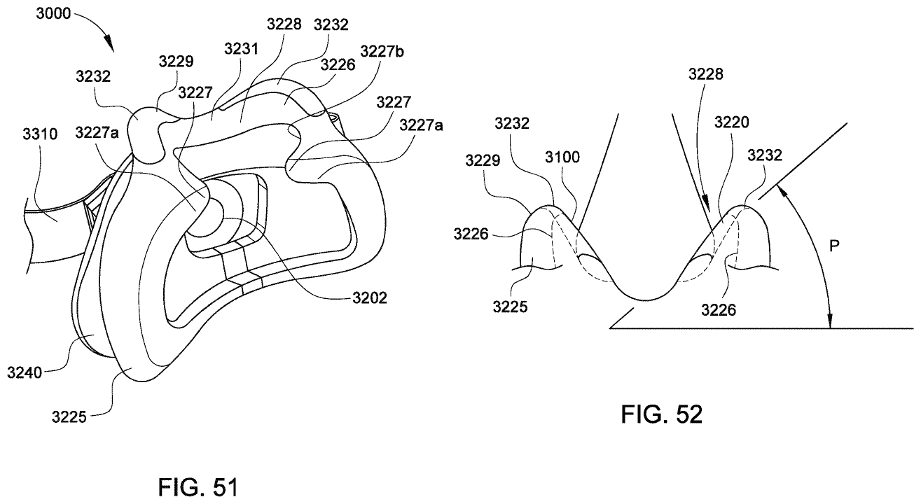

FIG. 51 shows a posterior perspective view illustration of a patient interface according to another example of the present technology.

FIG. 52 is a schematic illustration showing sealing of the patient interface shown in FIG. 51 to a patient's nose.

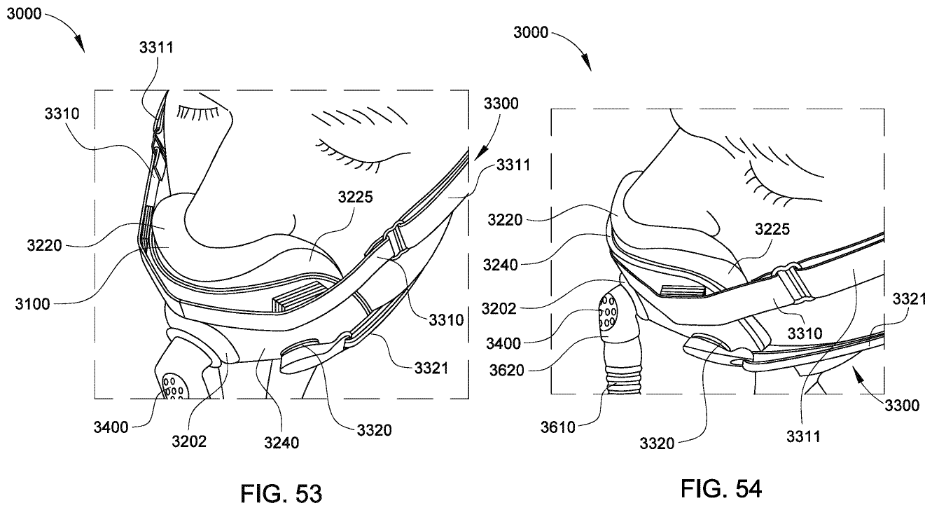

FIGS. 53-54 show anterior perspective view illustrations of the patient interface shown in FIG. 51 in use.

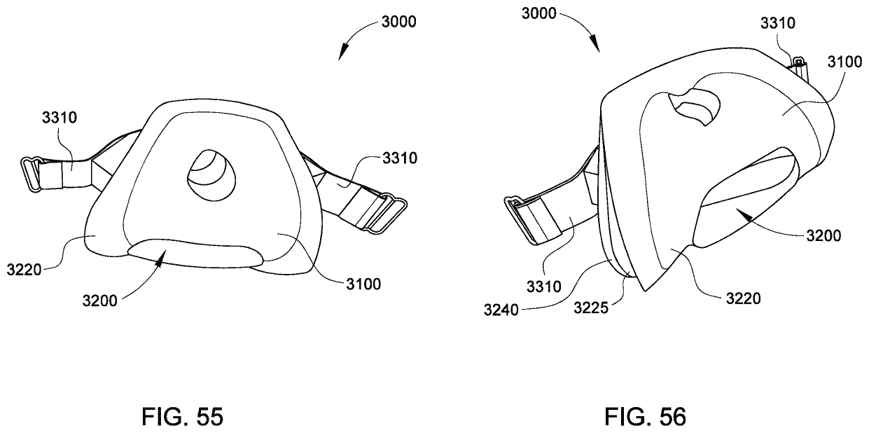

FIGS. 55-60 show illustrations of a patient interface and components thereof according to another example of the present technology

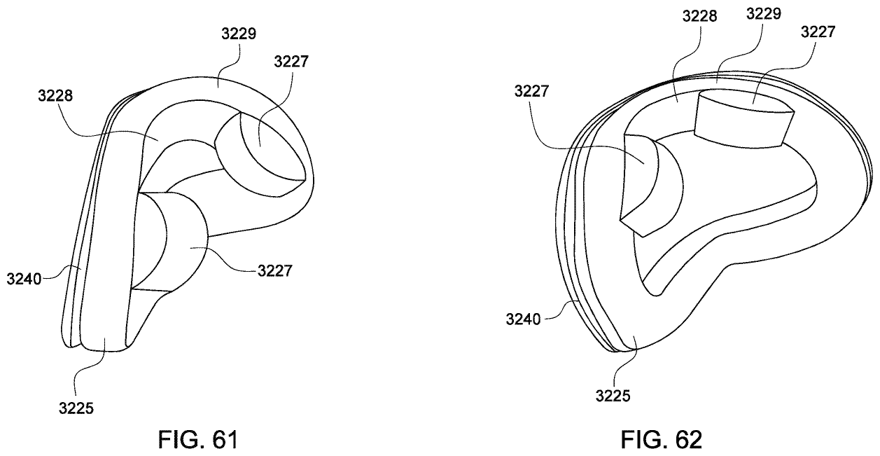

FIGS. 61-62 show illustrations of an undercushion and fascia portion of a patient interface according to another example of the present technology.

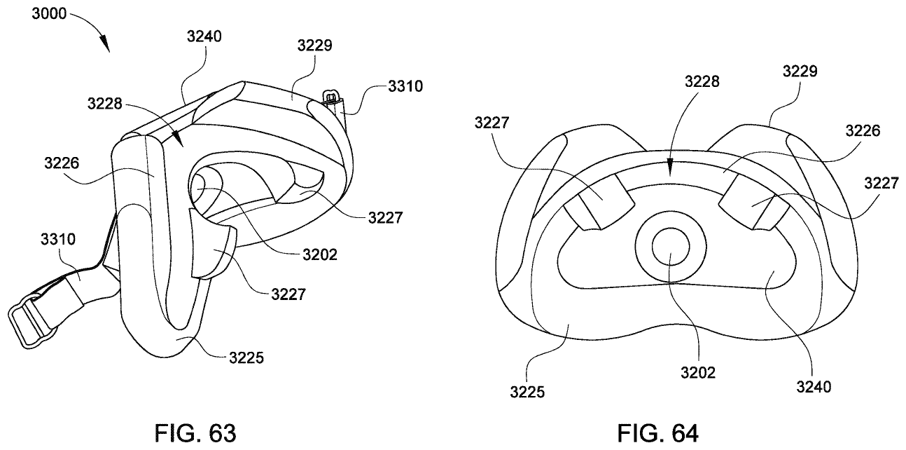

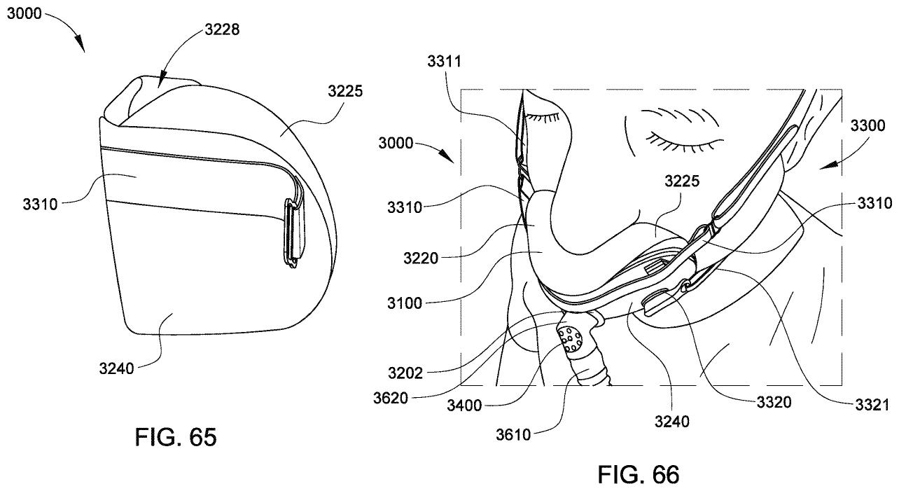

FIGS. 63-65 show illustrations of a patient interface and components thereof according to another example of the present technology.

FIG. 66 shows an anterior perspective view illustration of the patient interface shown in FIGS. 63-65 in use.

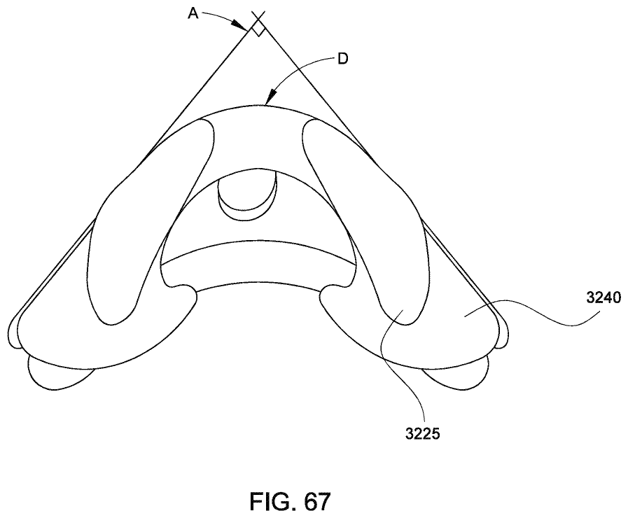

FIG. 67 shows an illustration of a portion of a patient interface according to another example of the present technology.

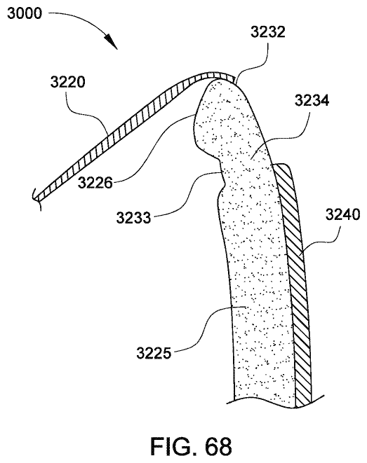

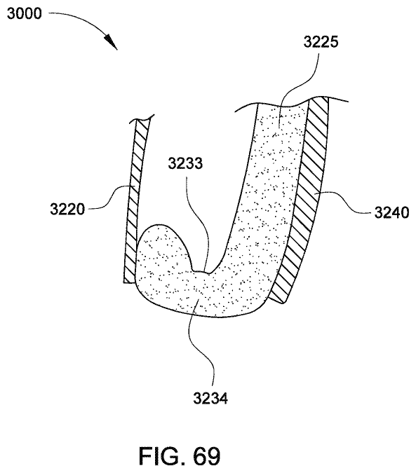

FIGS. 68 and 69 show illustrations of partial cross sections through a patient interface according to another example of the present technology.

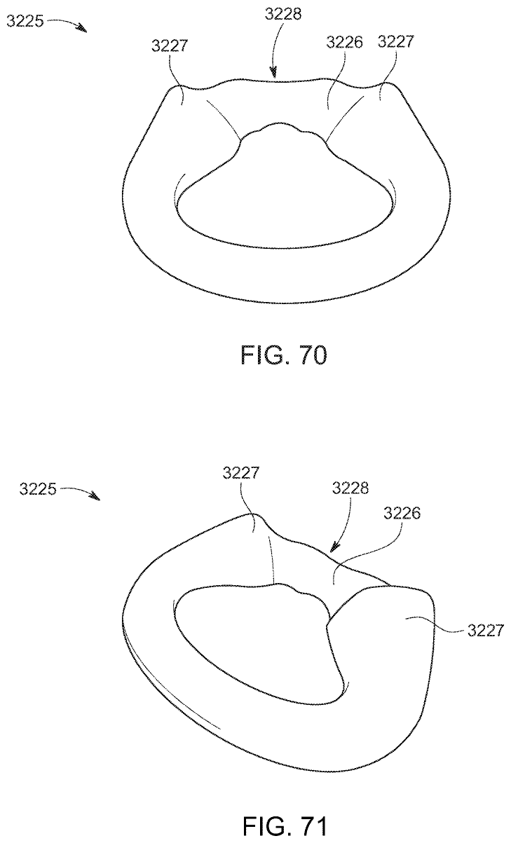

FIGS. 70 and 71 show views of an undercushion according to another example of the present technology.

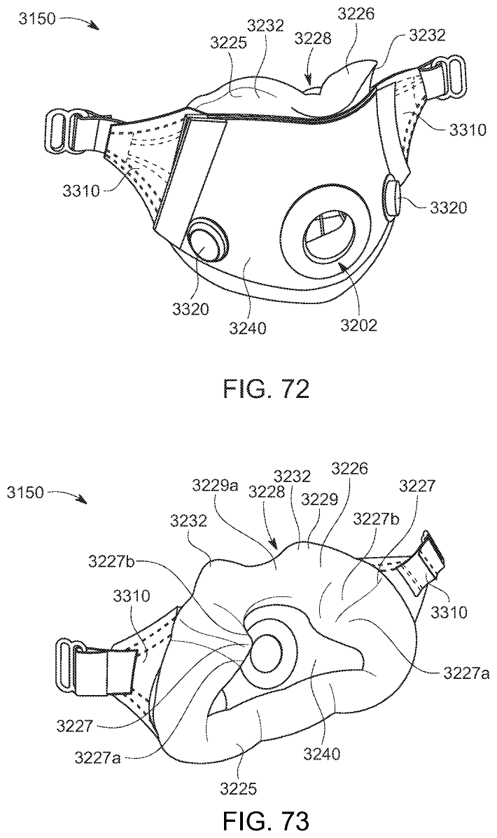

FIG. 72 shows an anterior view of a cushion module comprising the undercushion of FIG. 70, with a membrane portion omitted.

FIG. 73 shows a posterior view of the cushion module of FIG. 72, with a membrane portion omitted.

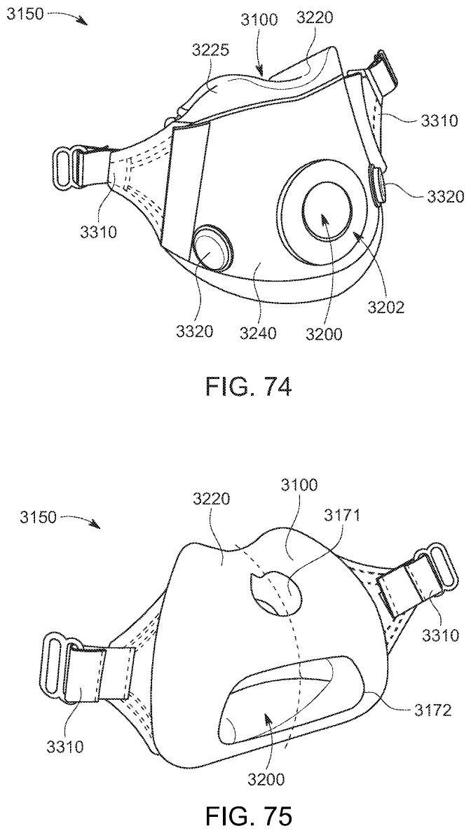

FIG. 74 shows an anterior view of the cushion module of FIG. 72 with membrane portion included.

FIG. 75 shows a posterior view of the cushion module of FIG. 72 with membrane portion included.

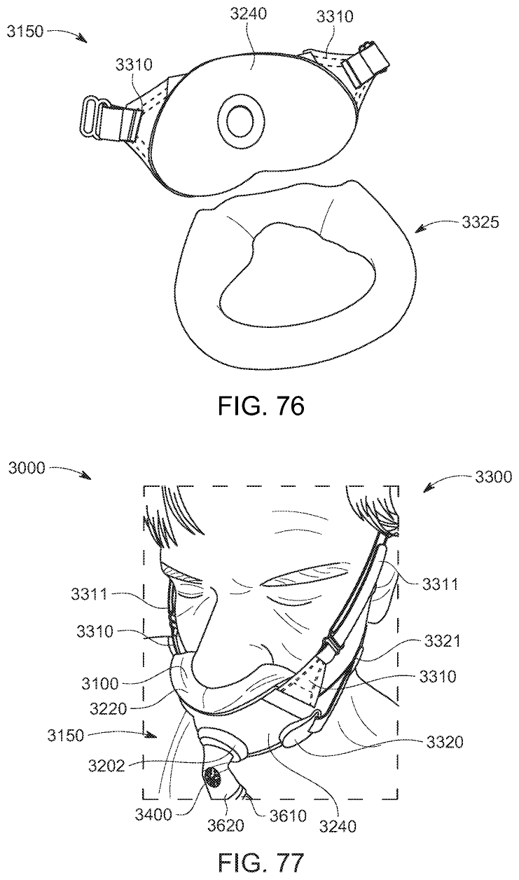

FIG. 76 shows a partially exploded view of the cushion module of FIG. 72 with membrane portion omitted.

FIG. 77 shows a anterosuperior view of a patient interface comprising the cushion module of FIG. 72, when worn by a patient.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and in some particular examples, a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3Z, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components (e.g., a cushion module 3150 together with the seal-forming structure 3100) in the sense that it or they can be replaced with different components, for example components of a different size. A cushion module 3150 may be the portion of the mask forming the plenum chamber 3200 and seal-forming structure 3100. A cushion module 3150 may be separable from other components of the patient interface 3000, or may not be separable from one or more other components, such portions of a positioning and stabilising structure 3300, a vent module, a short tube 3610 and/or a decoupling structure, as examples.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 2, 4, 6, 8 or at least 10 cmH₂O with respect to ambient.

The patient interface 3000 in accordance with one form of the present technology is constructed and arranged to be able to provide a supply of air at a positive pressure of at least 20 cmH2O with respect to ambient, for example up to 30 cmH2O or up to 40 cmH2O.

FIGS. 43-77 show various views of patient interfaces 3000 according to examples of the present technology, and components or portions thereof. In these examples the patient interfaces 3000 comprise a plenum chamber 3200 pressurisable to a therapeutic pressure of at least 6 cmH2O above ambient air pressure. The plenum chamber 3200 has at least one plenum chamber inlet port 3202 sized and structured to receive the flow of air at the therapeutic pressure for breathing by the patient. In these examples the plenum chamber 3200 of each patient interface is partially formed by a fascia portion 3240 of the patient interface 3000. The plenum chamber inlet port 3202 may be formed in the fascia portion 3240, for example as shown in FIGS. 51, 53, 54, 63, 64, 66 and 72-77. In many other drawings of the fascia portion 3240 the plenum chamber inlet port 3202 is not shown or not labelled. The fascia portion 3240 may at least partially form an anterior side of the patient interface 3000, as shown in FIGS. 53, 54, 66 and 72-77 for example.

The patient interface 3000 may further comprise at least one pair of headgear connectors configured to connect to a positioning and stabilising structure 3300 (see FIGS. 52, 53, 66 and 77) of the patient interface 3000. The headgear connectors may be connected to the fascia portion 3240. In the illustrated examples the patient interface 3000 comprises a pair of superior headgear connector portions 3310 and a pair of inferior headgear connector portions 3320. In other examples there may be only one pair of headgear connectors (e.g. a two-point headgear connection).

In the example shown in FIGS. 70-77, the headgear connectors are substantially as described separately below with reference to FIGS. 29-36.

Also with reference to FIGS. 43-77, the patient interfaces 3000 each further comprise a seal-forming structure 3100 partially forming the plenum chamber 3200. The seal-forming structure 3100 may be attached to the fascia portion 3240. The seal-forming structure 3100 is constructed and arranged to form a seal with a region of the patient's face surrounding an entrance to the patient's airways in use. The seal-forming structure 3100 has at least one hole therein such that the flow of air at the therapeutic pressure is delivered to an entrance to the patient's nares and the patient's mouth. The seal-forming structure 3100 is constructed and arranged to maintain said therapeutic pressure in the plenum chamber throughout the patient's respiratory cycle in use.

In some examples of the present technology, the seal-forming structure 3100 comprises an undercushion 3225. The seal-forming structure 3100 may further comprise a membrane portion 3220 connected to the undercushion 3225 and/or the fascia portion 3240 and which is configured to form a seal to at least a pronasale region and nasal alae of the patient's nose. In this example the seal-forming structure 3100 is further configured to form a seal around the patient's mouth. The seal-forming structure 3100 may be configured to inflate to form some or all of the seal. The seal-forming structure 3100 may additionally or alternatively be pressed against the patient's face to form the seal in one or more regions.

### 5.3.1 Cushion Module

In the examples shown in FIGS. 43-77, the fascia portion 3240, the undercushion 3225 and the membrane portion 3220 may together form a cushion module 3150. The cushion module 3150 may be combined with other components such as a positioning and stabilising structure 3300 (e.g. headgear) and a connector 3620 and/or short tube 3610 for fluid connection to an air circuit, to form a patient interface 3000. These components are shown in FIGS. 53, 54, 66 and 76 by way of example. The cushion module 3150, and therefore the fascia portion 3240, the undercushion 3225 and the membrane portion 3220, may be made available in multiple shape/size options, to fit a range of patient face shapes and sizes. Further details of cushion modules 3150 will be described below with reference to FIGS. 8-42.

FIGS. 8 and 9 show a patient interface 3000 according to another example of the present technology. FIGS. 10-16 show view of components of the patient interface 3000. FIGS. 17-42 show further patient interface components or patient interfaces 3000. The examples shown in FIGS. 8-42 will be described below in detail. It is to be understood that any of the features of the examples shown in, or described with reference to, FIGS. 8-42, may be combined with any of the features shown in, or described with reference to, FIGS. 43-77.

The patient interface 3000 comprises a cushion module 3150 forming a plenum chamber 3200 pressurisable to a therapeutic pressure of at least 4 cmH₂O above ambient air pressure. The plenum chamber 3200 includes a plenum chamber inlet port 3244, shown in FIG. 10, which is sized and structured to receive a flow of air at the therapeutic pressure for breathing by a patient.

The patient interface 3000 further comprises a seal-forming structure 3100 partially forming the cushion module 3150. The seal-forming structure 3100 may be constructed and arranged to form a seal with a region of the patient's face surrounding an entrance to the patient's airways. The seal-forming structure 3100 may have a hole therein such that the flow of air at said therapeutic pressure is delivered to at least an entrance to the patient's nares. In the examples shown in FIGS. 8-77, the seal forming structure comprises a nasal hole 3171 to provide the flow of air to the patient's nasal airways, and an oral hole 3172 to provide the flow of air to the patient's mouth. The seal-forming structure 3100 may be constructed and arranged to maintain the therapeutic pressure in the plenum chamber 3200 throughout the patient's respiratory cycle in use.

The patient interface 3000 further comprises a vent 3400. The vent 3400 may allow a continuous flow of gases exhaled by the patient from an interior of the plenum chamber 3200 to ambient. The vent 3400 may be sized and shaped to maintain the therapeutic pressure in the plenum chamber 3200 in use.

The patient interface 3000 in this example may be configured to allow the patient to breathe from ambient through their mouth in the absence of a flow of pressurised air through the plenum chamber inlet port 3244. For example, the patient faced 3000 may comprise an anti-asphyxia valve (AAV).

The cushion module 3150 may comprise the seal-forming structure 3100 and the fascia portion 3240 described below, and may also comprise further components or portions, such as an undercushion 3225 in the case of the examples shown in FIGS. 43-77.

The seal-forming structure 3100 in the examples shown or described with reference to FIGS. 8-42 may comprise a membrane portion 3220 configured to engage the patient's face in use. The membrane portion 3220 may be attached to the fascia portion 3240. For example, the membrane portion 3220 may be attached to a periphery of the fascia portion 3240.

The membrane portion 3220 may be configured to form a seal to at least a pronasale region and the nasal alae of the patient's nose. A patient interface 3000 having this configuration may be known as an ultracompact full face (UCFF) mask. As labelled in FIG. 11 and shown in other drawings, the membrane portion 3220 may comprise a nasal hole 3171 through which air can flow to both the patient's nares in use. In other examples the membrane portion 3220 or other seal-forming structure 3100 may comprise a pair of nasal holes 3171, each shaped and positioned to provide a flow of air to a respective one of the patient's nares. As shown in particular in FIG. 11 the membrane portion 3220 may comprise an oral hole 3172 through which air can flow to the patient's mouth, in use. In some examples, the membrane portion 3220 may comprise a single hole shaped and sized to seal around both the nasal airways and the mouth.

In some examples the nasal hole 3171 and oral hole 3172 are oriented at substantially 90 degrees to each other. In other examples they may be oriented between 70-90 degrees to each other, for example.

The membrane portion 3220 may be formed at least partially from a textile material and may be air-impermeable. The membrane portion 3220 may comprise a textile layer and an air-impermeable layer, such as a silicone or TPU film layer. In other examples the membrane portion 3220 may be formed from an elastomeric material such as silicone or a thermoplastic elastomer. In some examples the membrane portion 3220 may comprise an overall thickness of 0.3 mm. For example, the membrane portion 3220 may comprise a textile layer having a thickness of 0.27 mm and a silicone layer of 0.03 mm applied to its underside (interior of plenum chamber 3200). The textile may be knitted, e.g. from synthetic fibres. The membrane portion 3220 may be knitted to allow it to stretch to readily form to the patient's face in use. In other examples, the membrane portion 3220 may be thicker, for example 0.5 mm overall.

The cushion module 3150 may comprise at least one pair of headgear connection portions connected to the fascia portion 3240 and configured to connect to a positioning and stabilising structure 3300 of the patient interface 3000. In the examples shown or described with reference to FIGS. 8-36, for example, the patient interface 3000 or cushion module 3150 thereof may comprise a pair of superior headgear connection portions 3310 connected to the fascia portion 3240 and a pair of inferior headgear connection portions 3320 connected to the fascia portion 3240. In the examples shown in FIGS. 8-28, each of the superior headgear connection portions 3310 comprises a curved arm. Each of the inferior headgear connection portions 3320 comprises a magnetic connector. As shown in FIGS. 8 and 9 a superior strap portion 3311 of the positioning and stabilising structure 3300 may attach to each superior headgear connection portion 3310 and an inferior strap portion 3321 of the positioning and stabilising structure 3300 may attach to each of the inferior headgear connection portions 3320. The superior strap portions 3311 and inferior strap portions 3321 of the positioning and stabilising structure 3300 may be connected to a crown strap portion 3360, as shown in FIG. 9 for example. The crown strap portion 3360 may engage the posterior of the patient's head, remaining in a stable position and providing an anchor which the superior strap portions 3311 and inferior strap portions 3321 connect to, and transmit tension to, in use.

The superior headgear connection portions 3310 are not shown in many of the drawings showing the fascia portion 3240, for clarity.

### 5.3.1.1 Fascia Portion

With reference to FIGS. 8-16, the patient interface 3000 in this example comprises a fascia portion 3240. The fascia portion 3240 may partially form the plenum chamber 3200. The fascia portion 3240 may form the front of the cushion module 3150, and in some examples may comprise a plenum chamber inlet port 3244 and/or connections to a positioning and stabilising structure 3300. The fascia portion 3240 may at least partially form an anterior side of the cushion module 3150. For example, the fascia portion 3240 may form most of a non-patient facing side of the cushion module 3150. The seal-forming structure 3100 may be attached to the fascia portion 3240. For example, the seal-forming structure 3100 may be attached to a patient-facing side of the fascia portion 3240. As shown in FIGS. 15 and 16 in particular, the fascia portion 3240 may comprise a curved shape and may be curved at least partially towards a posterior direction on lateral sides of the fascia portion 3240 in use. The fascia portion 3240 may be curved to follow the shape of the patient's face. For example, the fascia portion 3240 may be curved to wrap around the patient's face on either lateral side of the mid-sagittal plane of the user's head.

As shown in FIG. 15, the fascia portion 3240 may have a non-zero negative first principal curvature P1 and a second principal curvature P2 which is less than the first principal curvature P1. In some examples the second principal curvature P2 may be less than the first principal curvature P1 in the sense that the second principal curvature P2 may be zero. The outer periphery of the fascia portion 3240 may be reminiscent of a hyperbolic paraboloid in some examples. In some examples the fascia portion 3240 comprises a single curved surface to provide a structure being somewhat rigid in the vertical direction but more flexible in the horizontal directions to allow it to conform to the patient's face. The curved shape may provide for a low profile mask that closely follows the shape of the patient's face from left to right. The fascia portion 3240 may lie close to tangent with the patient's face in use, for example.

The fascia portion 3240 may be formed from a flexible material. In some examples the fascia portion is formed at least partially from one or more foam and/or textile materials. In some examples, the fascia portion 3240 is formed from foam covered with a textile material on at least a non-patient facing side thereof. In some examples the fascia portion 3240 comprises a layer of foam covered by layers of textile material on two sides thereof (such as a patient-facing side and a non-patient facing side). The fascia portion 3240 may be airtight, for example by having one or more airtight layers, such as an airtight foam or an air-impermeable coating or film layer. In other examples the fascia portion 3240 may be formed from spacer fabric. In examples, the fascia portion 3240 may be formed from other synthetic materials that can be thermoformed into shape.

FIGS. 17-28 show views of further examples of cushion modules 3150 and fascia portions 3240. In each of these examples the fascia portion 3240 also comprises a curved shape as described above.

In the examples shown in FIGS. 8, 9, 12-14, and 29-36, the cushion module 3150 comprises a deformable adjustment member 3242 attached to the fascia portion 3240. The deformable adjustment member 3242 may be structured and arranged to be selectively adjusted by the patient to adjust a curvature of the fascia portion 3240. In these particular examples, the deformable adjustment member 3242 comprises a deformable metal (e.g. aluminium) strip spanning from one lateral side of the fascia portion 3240 to the other lateral side. The deformable metal strip may span between locations at or proximate the joints between the first headgear connection portions 3310 and the fascia portion 3240. In the example shown in FIGS. 8 and 9 there is one deformable adjustment member 3242 and in the example shown in FIGS. 29-36 there are two deformable adjustment members 3242. The deformable adjustment member 3242 may alternatively be a plastically deformable polymer instead of a metal strip. As shown in FIGS. 13 and 14, the deformable adjustment member 3242 allows the patient to adjust with width of the cushion module 3150, by adjusting the curvature of the fascia portion 3240, between a wider configuration (shown in FIG. 13) and a narrower configuration (shown in FIG. 14). This may advantageously allow the patient to tailor the shape of the cushion module 3150 to their face to help achieve a good fit. The deformable adjustment member 3242 may be provided to a superior portion of the fascia portion 3240 to enable it to adjust primarily to the curvature of the fascia portion 3240 proximate the patient's nose. The deformable adjustment member 3242 may be provided either together or separately with biasing mechanism(s) discussed below. For example, a fascia portion 3240 may be biased outward to splay the seal-forming structure 3100 outwards but the deformable adjustment member 3242 may set a limit on the width of a superior portion of the fascia portion 3240, to help resolve leaks proximate the nose.

### 5.3.1.1.1 Bias of Fascia Portion

In each of the examples shown in FIGS. 8-28, the fascia portion 3240 may be biased away from the curved shape towards a flatter shape than the curved shape, to provide tautness to the seal-forming structure 3100 in use. This may advantageously provide for a good seal with a low risk of leaks and may be comfortable for the patient, or at least more so than a seal-forming structure 3100 that is not urged to be taut.

In some examples, such as the examples shown in FIGS. 8-24 the fascia portion 3240 may be first formed into a formed shape having less curvature than the curved shape. However, after attachment of the seal-forming structure 3100 to the fascia portion 3240 the seal-forming structure 3100 may hold the fascia portion 3240 in the curved shape, and the fascia portion 3240 may be biased away from the curved shape by a tendency to return to the formed shape. That is, the fascia portion 3240 is formed during manufacturing such that the fascia portion 3240 is less curved than it is intended to be during use. The seal-forming structure 3100 may be attached to the fascia portion 3240 during a subsequent manufacturing step. The seal-forming structure 3100 may be sized to pull the sides of the fascia portion 3240 medially after the seal-forming structure 3100 is attached to the fascia portion 3240, pulling the fascia portion 3240 into a shape that is more curved than the original formed shape of the fascia portion 3240. The fascia portion 3240 may be biased back towards the less curved, formed shape, after which the sides of the fascia portion 3240 may tend to pull laterally on the seal-forming structure 3100. This may tend to splay the seal-forming structure 3100 outwards in the directions indicated by the axis S in FIG. 21, which may advantageously provide for a taut seal-forming structure 3100 that may be less likely to leak than a flaccid seal-forming structure 3100.

The fascia portion 3240 may be thermoformed into the formed shape, for example.

In another form of the present technology, shown in FIGS. 25-28 for example, the cushion module 3150 may comprise a resilient member 3260 biasing the fascia portion 3240 away from the curved shape. In this example the fascia portion 3240 may be formed into the curved shape and biased towards a less curved/flatter shape by the resilient member 3260. FIG. 25 shows the cushion module 3150 formed by membrane portion 3220 and fascia portion 3240. The membrane portion 3220 forms the seal-forming structure 3100 in this example. FIG. 25 also shows separately the resilient member 3260.

As shown in FIGS. 26-28, the resilient member 3260 is in this example provided interior to the plenum chamber 3200. The resilient member 3240 may engage the fascia portion along a connection between the fascia portion and the seal-forming structure 3100.

In this example the resilient member 3260 is biased towards an annular shape, as shown in FIG. 25. The resilient member 3260 may be formed in the annular shape and deformed during provision to the interior of the plenum chamber 3200. The resilient member 3260 may be biased towards the annular shape in use to bias the fascia portion away from the curved shape in use. That is, the resilient member 3260 may be formed having a ring shape/annular shape as shown in FIG. 25 and may be resilient to tend to return to that annular shape upon deformation.

The resilient member 3260 may engage the fascia portion 3240 along a connection between the fascia portion 3240 and the seal-forming structure 3100, as shown in FIGS. 26-28. The resilient member 3260 may fit inside the cushion module 3150 at the connection between the fascia portion 3240 and the seal-forming structure 3100, which may not lie on a circular curve. For example, the fascia portion 3240 may have the shape of a hyperbolic paraboloid or a parabolic cylinder. Similarly, in some examples the seal-forming structure 3100 or the membrane portion 3220 forming the seal-forming structure 3100 may also have the shape of a hyperbolic paraboloid or a parabolic cylinder. Additionally or alternatively, the connection, boundary, joint or seam (as the case may be) between the fascia portion 3240 and the seal-forming structure 3100 may be a curve having the shape of the boundary of a hyperbolic paraboloid or a parabolic cylinder. The deformation of the resilient member 3260 into this non-circular shape may cause the resilient member 3260 to exert a restoring force back onto the cushion module 3150 or at least the fascia portion 3240 or seal-forming structure 3100 thereof. Accordingly, the resilient member 3260 may bias the fascia portion 3240 away from its curved in-use shape towards a flatter shape than the curved shape to provide tautness to the seal-forming structure in use.

A seal-forming structure 3100 that presents to a patient without creases may have a higher chance of attaining a good seal as creases can often lead to leak paths. Having a component that can apply a tension to the seal-forming structure 3100 may help maintain a seal during dynamic sleeping as the applied tension will continuously be acting on any loose/creased regions that may form.

In some examples, the resilient member 3260 may splay the seal-forming structure 3100 out laterally and also splay the seal-forming structure 3100 out in superior and inferior directions, as indicated by the four arrows shown in FIG. 28.

The resilient member 3260 may formed from a polymer material such as nylon, ABS, TPU, silicone or from stainless steel or any other suitable material. The resilient member 3260 may, for example, be moulded. Alternatively the resilient member may be drawn or extruded, cut and its ends may be joined to form the annular shape. In some examples the resilient member 3260 may be elongate (e.g. having ends not joined. In some examples the resilient member 3260 may biased towards a straight shape, or at least a straighter shape than it may take when assembled in the cushion module 3150. The resilient member 3260 may be inherently springloaded.

The resilient member 3260 may have a diameter of 90 mm, in one example and may be formed with a round cross section having a diameter of 1.5 mm, when formed from a relatively stiff polymer such as nylon or ABS.

In some examples, the fascia portion 3240 may be biased to tauten then seal-forming structure 3100 or membrane portion 3220 with multiple mechanisms. For example, the fascia portion 3240 may have both an inherent bias towards a formed shape, and also a separate resilient member 3260 providing additional bias.

In other examples, the resilient member 3260 may be applied externally to the fascia portion 3240 instead of internally within the plenum chamber 3200. That said, providing the resilient member 3260 internally may enable it to act directly on the seal-forming structure 3100 and may also make it more discrete.

### 5.3.1.2 Seal Support Portions

In the examples shown in FIGS. 17-28, the seal-forming structure 3100 further comprises, in addition to the membrane portion 3220, a pair of seal support portions 3250. Each may be provided to a respective lateral side of the fascia portion 3240 and may project in an at least partially medial direction from the fascia portion 3240. Each seal support portion 3250 may be configured to urge the membrane portion 3220 against the patient's face in use. The seal support portions 3250 may function as undercushions.

FIGS. 17-19 show the fascia portion 3240 and seal support portions 3250 in isolation with membrane portion 3220 and other components not shown. FIGS. 20, 21, 23 and 24 show cushion modules 3150 with membrane portion 3220 included and the seal support portions 3250 depicted by hidden detail. FIG. 22 shows the seal support portions 3250 through the oral hole 3172 of the cushion module 3150.

As illustrated, each of the pair of seal support portions 3250 may project from at or proximate the periphery of the fascia portion 3240. In these particular examples, each of the pair of seal support portions 3250 may project from proximate a peripheral edge of the fascia portion 3240 but may be spaced from the peripheral edge by a spacing. This may advantageously help with manufacturing the cushion module 3150 and may allow for air flow between the membrane portion 3220 and each seal support portion 3250, which may help inflate the membrane portion 3220 in any localised locations or instances (transient or otherwise) in which the seal support portion 3250 are not in engagement with the patient's face. The spacing may be in the range of 0.5-5 mm, such as within the range of 1-3 mm, for example. In other examples, there is no spacing between the seal support portions 3250 and the peripheral edge of the fascia portion 3240. In such examples air within the plenum chamber 3200 may still tend to flow between the seal support portions 3250 and the membrane portion 3220, after which the membrane portion 3220 may still inflate over the seal support portions 3250, when the plenum chamber 3200 becomes pressurised.

Each of the pair of seal support portions 3250 may be structured to urge the membrane portion 3220 against a respective one of the patient's cheeks in use. Additionally, in the illustrated examples, each of the pair of seal support portions 3250 is structured and arranged to urge the membrane portion 3220 to seal at or proximate a respective one of the alar crest points on the patient's face. This may be a difficult place to seal to and the seal support portions 3250 may advantageously help achieve a good seal at these locations and/or more generally around an inferior periphery of the user's nose. In the example shown in FIG. 24, each of the pair of seal support portions 3250 comprises a medial projection 3253 structured and arranged to urge the membrane portion 3220 towards the user's face proximate a respective one of the alar crest points in use. In other examples, such as the example shown in FIGS. 17-23, the seal support portions 3250 may project towards the alar crest points more generally. In these examples, each of the pair of seal support portions 3250 comprises an inferior portion 3251 having a concave medial edge. The inferior portions 3251 may urge the membrane portion 3220 against the patient's cheeks. Each of the pair of seal support portions 3250 may additionally or alternatively comprise a superior portion 3252 having a convex medial edge. The superior portions 3252 may urge the membrane portion 3220 to seal at or proximate the inferior periphery of the patient's nose. FIG. 24 shows an alternative example in which the superior portion 3252 has a concave medial edge structured and arranged to lie proximate to and follow the shape of the patient nasal ala. This arrangement may provide less medially directed force on the patient's nasal ala than the example shown in FIGS. 17-23, which may be better performing or more preferable for some patients.

The seal support portions 3250 may be at least partially formed from foam. In some examples one or both sides of the seal support portions 3250 may be covered with a textile layer. In the illustrated examples, the seal support portions 3250 are formed from the same material as the fascia portion 3240. In some examples, the fascia portion 3240 is thermoformed to shape (e.g. into a curved shape). In some examples, the seal support portions 3250 may not be thermoformed to shape. This may make them more comfortable on the patient's face and/or more compliant to the shape of the patient's face. In other examples, the seal support portions 3250 may be thermoformed to shape.

In examples, the seal support portion 3250 may be integrally formed with the fascia portion 3240 or may be formed separately and then adhered, welded, otherwise bonded, or sewn to the fascia portion 3240, in examples.

In some examples, the seal support portions 3250 may be formed from a 4 mm thick layer of foam covered with a textile material on one or both sides. In other examples the seal support portion 3250 may be formed from foam having a thickness of within the range of 3.5-4.5 mm or within the range of 3-5 mm.

The seal support portion 3250 may project both medially and posteriorly in use. In some examples the edges of the seal support portions 3250, for example the concave and/or convex edges of the inferior portions 3251 and superior portions 3252 of the seal support portions 3250 may be urged against the membrane portion 3220 even when the patient interface 3000 is not in use. This may help keep the membrane portion 3220 taut and therefore may advantageously aid in maintaining a good seal during use.

In some examples, the seal support portion 3250 may be stiffer in the inferior portions 3251 than in the superior portions 3252, for example as a result of a longer cantilevered length in the superior portions 3252 than in the inferior portions 3251. The seal support portions 3250 may effectively form cantilever structures of varying length which are relatively flexible and soft in some regions, and firm in others. This variability in stiffness coincides with different regions of the face and their ability to endure load. The seal support portions 3250 reacts against the face from headgear input and remains comfortable over its entire length while allowing the seal to perform its pressurised sealing function.

The membrane portion 3220 may overhang each seal support portion 3250, as illustrated in FIGS. 20, 21, 23 and 24. This may help the membrane portion 3220 seal proximate the nasal and oral holes of the membrane portion 3220 since there is no impediment to the therapeutic pressure in the plenum chamber 3200 acting on the interior surface of the membrane portion 3220.

### 5.3.1.3 Flexible Headgear Connection Portions

FIGS. 29-36 show a patient interface 3000 and cushion module 3150 thereof another example of the present technology. In this example the fascia portion 3240 is formed from a flexible material and may be as described above with reference to other examples. The membrane portion 3220 may also be as described above with reference to other examples.

The patient interface 3000 in this example comprises a pair of superior headgear connection portions 3310 (which may be identified as first or lateral headgear connection portions in examples in which there is only one headgear strap connecting on each lateral side of the cushion module 3150) configured to connect to superior strap portions 3311 (which may be identified as first or lateral strap portions in two-point headgear connection examples) of a positioning and stabilising structure 3300 of the patient interface 3000. The superior headgear connection portions 3311 may each be formed from a flexible material and may each be attached to a non-patient facing side of the fascia portion 3240 proximate lateral sides of the fascia portion 3240.

In this example, and in other examples disclosed herein, the fascia portion 3240 comprises a peripheral edge formed by a superior edge portion 3245 and an inferior edge portion 3246 connected to each other by a pair of lateral edge portions 3247 at respective lateral sides of the fascia portion 3240. Each lateral edge portion 3247 comprises a lateralmost point.

In the particular example shown in FIGS. 29-36, each of the superior headgear connection portions 3311 is attached to the fascia portion 3240 medially of the lateral edge portion 3247. In this example, the superior headgear connection portions 3310 are structured and arranged to urge the fascia portion 3240 proximate the lateralmost point towards the patient's face in use. With reference to FIGS. 35 and 36, the fascia portion 3240 comprises a lateral portion 3248 on each lateral side thereof which the superior strap portion 3310 urges towards the patient's face. Advantageously, this may allow for good engagement of the seal-forming structure 3100 with the patient's face. The mostly posteriorly directed tension in the superior strap portions 3311 of the positioning and stabilising structure 3300 may become translated into a medially directed force applied to the fascia portion 3240 and then, in turn, to the seal-forming structure 3100. The superior headgear connection portions 3310 may also be identified as “wings”. The wings may extend in a partially posterior and partially lateral direction substantially tangentially from the non-patient facing surface of the fascia portion 3240. They may be attached to the fascia portion 3240 and extend substantially tangentially from the surface of the fascia portion 3240 such that they are substantially flush with the non-patient facing surface of the fascia portion 3240.

Each of the superior headgear connection portions 3310 may be attached to the fascia portion 3240 proximate both the superior edge portion 3245 and the inferior edge portion 3246 of the peripheral edge of the fascia portion 3240. In particular, in the illustrated example, each of the superior headgear connection portions 3310 is attached to the fascia portion 3240 at a joint 3315 lying along the non-patient facing side of the fascia portion 3240 from at or proximate the superior edge portion 3245 to at or proximate the inferior edge portion 3246 of the peripheral edge of the fascia portion 3240. This may allow the superior headgear connection portions 3310 to pull the fascia portion 3240 into contact with the user's face across the entire height of the cushion module 3150, encouraging a good seal, and may also be aesthetically pleasing and may increase perceived comfort, which may encourage greater patient compliance.

Each of the superior headgear connection portions 3310 may be able to pivot with respect to the fascia portion 3240 along the joint 3315. This flexibility in the headgear connections may advantageously help to avoid unwanted headgear forces transferring back onto the cushion module 3150, such as which may occur during side sleeping. The wings may work in unison with the headgear straps so that the patient interface 3000 as a whole lies close to the face/cheeks. This may provide for a low profile patient interface 3000, which may keep any risk of lateral shunting during side sleeping low. In particular, the flexible textile construction of the headgear connections may help impede mask shunting. For example, the flexible textile reduces the ability of the connection portions to transfer forces back towards the fascia portion 3240 to shunt the mask forwards. Also, the lateral flexibility of the headgear straps may advantageously reduce their ability for the connection portions to shunt the mask sideways.

The superior headgear connection portions 3310 may only contact the face towards a narrower distal end where the headgear straps 3311 attach. They effectively bridge the gap between cushion module 3150 and cheek.

With reference to FIGS. 33 and 34 in particular, each of the superior headgear connection portions 3310 comprises a superior edge 3312, an inferior edge 3313 and a superior strap connection point 3314 (which may be identified as a first or lateral strap connection point in the case of a cushion module 3150 having only two headgear connections). Each superior strap connection point 3314 may be configured to attach to a respective superior strap portion 3311 of the positioning and stabilising structure 3300. In this example, the superior edge 3312 and inferior edge 3313 of each of the superior headgear connection portions 3310 converge towards each other towards the superior strap connection point 3314.

The superior edge 3312 of each of the superior headgear connection portions 3310 may lie in use substantially tangential to the superior edge portion 3245 of the peripheral edge of the fascia portion 3240. Additionally or alternatively, the inferior edge of each of the superior headgear connection portions 3310 may lie in use substantially tangential to the inferior edge portion 3246 of the peripheral edge of the fascia portion 3240. Accordingly, the outer shape of each superior headgear connection portion 3310 (e.g. the superior edges 3312 and inferior edges 3313) follows the outer peripheral shape (e.g. the superior edge portion 3245 and inferior edge portion 3246) of the fascia portion 3240, e.g. in a tangential manner. This may allow the superior headgear connection portions 3310 to pull the fascia portion 3240 into contact with the user's face across the entire height of the cushion module 3150, encouraging a good seal, and may also be aesthetically pleasing which may increase perceived comfort and/or greater patient compliance.

As shown in FIGS. 29-36, the cushion module 3150 further comprises a pair of inferior headgear connection portions 3320 configured to connect to inferior strap portions 3321 of the positioning and stabilising structure 3300. In this example each inferior headgear connection portion 3320 comprises a magnetic headgear connection point provided to the fascia portion 3240 to which a respective inferior strap portion 3321 is able to magnetically attach.

FIGS. 37 and 38 show a patient interface 3000 according to another example of the present technology. In this example there are only two points of headgear connection to the cushion module 3150. The cushion module 3150 comprises a pair of lateral headgear connection portions 3330 extending posteriorly of the seal-forming structure 3100 at lateral sides of the cushion module 3150. The lateral headgear connection portions 3330 may be configured to connect to lateral strap portions 3331 of a positioning and stabilising structure 3300. The lateral headgear connection portions 3330 in this example are each formed from a flexible material. The cushion material 3150 is configured to be supported in position on the patient's face in use by the lateral strap portions 3330 of the positioning and stabilising structure 3300 in the absence of any other strap portions of the positioning and stabilising structure 3300. The lateral headgear connection portions 3330 could also be identified more generally as headgear connection portions. Each of the lateral headgear connection portions 3330 in this example comprises a slot to which the respective lateral strap portion 3331 of the positioning and stabilising structure 3300 is able to connect in use.

The fascia portion 3240 comprises a superior edge portion 3245 and an inferior edge portion 3246 defining a height of the fascia portion 3240, and each of the lateral headgear connection portions 3330 comprises a superior edge 3332 and an inferior edge 3333 defining a height of the respective lateral headgear connection portion 3330.

As illustrated, each of the lateral headgear connection portions 3330 is attached to the fascia portion 3240 proximate both the superior edge portion 3245 and the inferior edge portion 3246 of the peripheral edge of the fascia portion 3240. The height of each lateral headgear connection portion 3330 may be at least half the height of the fascia portion 3240. In some examples the height of each lateral headgear connection portion 3330 may be at least two-thirds the height of the fascia portion 3240. In the illustrated example, the superior edges 3332 of the respective lateral headgear connection portions 3330 are located proximate the superior edge portion 3245 of the fascia portion 3240. Likewise, the inferior edges 3333 of the respective lateral headgear connection portions 3330 are located proximate the inferior edge portion 3246 of the fascia portion 3240. In this example, the height each lateral headgear connection portion 3330 is substantially the same as the height of the fascia portion 3240. Each of the lateral headgear connection portions 3330 is connected to the fascia portion 3240 substantially continuously along the height of the respective lateral headgear connection portion 3330. This arrangement may advantageously provide for an even transfer of force to the cushion module 3150 from the lateral strap portions 3331 of the positioning and stabilising structure 3300, encouraging a sufficient and stable seal.

The flexibility in the lateral headgear connection portions 3330 may provide similar advantages to the advantages described above in relation to the superior headgear connection portions 3310 shown in FIGS. 29-36, such as the ability to transfer tension in headgear straps into posteriorly and/or superiorly directed force on the cushion module 3150, without also transferring unwanted anteriorly directed forces from the headgear straps to the cushion module 3150, which may occur during movement or side sleeping, for example. Each of the lateral headgear connection portions 3330 may be able to pivot laterally and medially with respect to the fascia portion 3240. This may further decouple the cushion module 3150 from unwanted forces received by the headgear straps. The flexible lateral headgear connections 3330 may also advantageously provide a two-point connection to the cushion module 3150 that inherently allows the cushion module 3150 some freedom to pivot with respect to the positioning and stabilising structure 3300. This may advantageously allow for the patient interface 3000 to tolerate variation in face shapes and sizes and therefore fit a large number of patients.

The lateral headgear connection portions 3330 may be integrally formed with the fascia portion 3240, in some examples. The fascia portion 3240 may be constructed as described elsewhere herein, for example formed from foam and/or textile materials. In other examples the lateral headgear connection portions 3330 may be bonded, welded, sewn or otherwise attached to the fascia portion 3240.

### 5.3.1.4 Frame Provided to Fascia Portion

FIGS. 39-42 show a cushion module 3150 according to another example of the present technology, and components thereof. The patient interface 3000 comprises a fascia portion 3240 formed from a flexible material and at least partially forming an anterior side of the cushion module 3150. The seal-forming structure 3100 is attached to the fascia portion 3240. The seal-forming structure 3100 may comprise a membrane portion 3220 as described elsewhere herein. The fascia portion 3240 may be as described elsewhere herein save for some differences described below.

In this example, the patient interface 3000 comprises a frame 3290 attached to the fascia portion 3240. The frame 3290 may be constructed and arranged to rigidise the fascia portion 3240. The frame 3290 may be in the form of a skeleton. As shown in FIG. 40, the frame is formed by a plurality of elongate portions connected to each other. The frame 3290 may also be identified as an exoskeleton.

As illustrated, the frame 3290 in this example comprises a pair of lateral headgear connection portions 3294 located on respective lateral sides of the cushion module 3150. The lateral headgear connection portions 3294 may be configured to connect to lateral strap portions 3331 of a positioning and stabilising structure 3300, which may be the positioning and stabilising structure 3300 as shown in FIGS. 37 and 38, by way of example only. In the example shown in FIGS. 39-42, the cushion module 3150 is configured to be supported in position on the patient's face in use by the lateral strap portions 3331 of the positioning and stabilising structure 3300 in the absence of any other strap portions of the positioning and stabilising structure 3300.

The frame 3290 may be constructed and arranged to impart an in-use shape to the fascia portion 3240. The frame 3290 may hold the shape of the fascia portion 3240 in use, for example in the manner of a splint. The frame 3290 may be flexible and resilient but may be stiffer than the fascia portion 3240. In examples, the frame 3290 may be formed from nylon, Hytrel, polypropylene, polyurethane (e.g. TPU), polycarbonate, bamboo, silicone, thermoplastic elastomer or another suitable material including any suitable thermoplastic resin, elastomer or fabric for example. In other examples the frame 3290 may be flexible and resilient and function as a spring.

With reference to FIG. 39, the fascia portion 3240 comprises a peripheral edge formed by a superior edge portion 3245 and an inferior edge portion 3245 connected to each other by a pair of lateral edge portions 3247 at respective lateral sides of the fascia portion 3240. With reference to FIGS. 40 and 41, the frame 3290 in this example comprises a peripheral portion having a shape corresponding to the peripheral edge of the fascia portion 3240.

Like some of the other fascia portions 3240 exemplified herein, the superior edge portion 3245 of the fascia portion 3240 curves posteriorly away from the mid-sagittal plane on either lateral side of the patient's face in use, the lateral edge portions 3247 curve posteriorly and inferiorly away from the superior edge portions 3245 and then inferiorly and anteriorly towards the inferior edge portion 3246, and the inferior edge portion 3246 curves medially back towards the mid-sagittal plane.

As illustrated in FIG. 41, which shows the cushion module 3150 and frame 3290 in an assembled configuration, the peripheral portion of the frame 3290. e.g. superior, inferior and lateral portions of the frame 3290, has a shape substantially following the peripheral edge of the fascia portion 3240. Each lateral strap connection portion 3294 of the frame 3290 connects between a superior portion 3291 of the frame 3290 and an inferior portion 3292 of the frame 3290. When the frame 3290 is attached to the fascia portion 3240, each lateral strap connection portion 3294 connects to the superior portion 3291 of the frame 3290 proximate a junction between the superior edge portion 3245 and a respective lateral edge portion 3247 of the peripheral edge of the fascia portion 3240. Similarly, each lateral strap connection portion 3294 connects to the inferior portion 3292 of the frame 3290 proximate a junction between the inferior edge portion 3246 and respective lateral edge portion 3247 of the peripheral edge of the fascia portion 3240. As illustrated in FIGS. 40 and 41, each lateral strap connection portion 3330 in this example comprises an elongate portion around which a respective lateral strap portion 3331 of the positioning and stabilising structure 3300 is able to wrap and be secured back to itself (for example with a hook and loop connection).

The fascia portion 3240 may be formed from a foam and/or textile material. And in the example illustrated in FIGS. 39-42 is formed from foam covered with a textile material on at least the non-patient facing side thereof. In this example the fascia portion 3240 comprises lateral pockets 3280 structured and arranged to receive and retain the frame 3290 in use. The pockets 3280 may be formed from a textile material, such as the same textile material covering a non-patient facing surface of the fascia portion 3240 and/or the same textile material forming the membrane portion 3220. As shown in FIG. 40, the frame 3290 may comprise a pair of lateral portions 3293, each being lateral of a respective one of the lateral strap connection portions 3294. Each lateral portion 3293 may be received in a respective one of the lateral pockets 3280 in use.

The pockets 3280 may each be formed by an additional layer of material attached to the non-patient facing side of fascia portion 3240. The additional layer of material may have substantially the same outer peripheral shape as the lateral portion of the fascia portion 3240. Each additional layer of material may comprise a lateral edge joined to the fascia portion 3240 along a respective lateral edge portion 3247 of the peripheral edge of the fascia portion. Additionally, each additional layer of material may comprise a medial edge which is not joined to the fascia portion 3240. In this example shown in FIG. 41, this forms a pocket 3280 opening medially to receive a respective lateral portion 3293 of the frame 3290. The pockets 3280 may be formed from any material suitable for retaining the frame 3290, such as nylon, polyester or silicone. The material may be stretchable in order to snuggly retain the frame 3290. The material may be attached to the fascia portion 3280 in any suitable manner, such as by gluing, welding, or otherwise bonding or sewing.

With reference to FIGS. 41 and 42, the cushion module 3150 in this example comprises a vent module 3402. The vent module 3402 is structured to be received in an anterior hole 3271 in the fascia portion 3240. The vent module 3402 in this example defines the plenum chamber inlet port 3244 and also provides a vent 3400. The vent 3400 may be formed by a plurality of holes provided circumferentially around the plenum chamber inlet port 3244. The vent module 3402 may be in the form of a ring. In some examples the vent module 3402 comprises a diffuser material through which the vent flow of gas passes prior to or during venting from the vent 3400. The vent module 3402 may be configured to attach to a connector, such as a swivel connector 3620 shown in FIGS. 8 and 9 at the plenum chamber inlet port 3244. The vent module 3402 may be provided to any of the examples of cushion modules 3150 described herein.

### 5.3.2 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with some examples of the present technology may be constructed from, or at least comprise, a soft, flexible, resilient material such as silicone.

In some forms the seal forming structure 3100 comprises an undercushion 3225, which may be formed from foam, and a membrane portion 3220, which may be at least partially formed from a textile material, as described further below. The seal-forming structure 3100 may be connected to the fascia portion 3240 and may partially form the plenum chamber 3200. It is to be understood that where the membrane portion 3220 is described as being formed from a textile material, it may comprise a non-textile layer, for example a silicone, polyurethane or other layer or film of other suitable material to provide airtightness/air-impermeability.

In certain forms of the present technology, a system is provided comprising more than one a seal-forming structure 3100, each being configured to correspond to a different size and/or shape range. The different seal-forming structures 3100 may be provided to different cushion module options. For example the system may comprise one form of a seal-forming structure 3100 suitable for a large sized head, but not a small sized head and another suitable for a small sized head, but not a large sized head. However, examples of the technology may be suitable for a large range of heads, and so may be used by patients having a relatively large head and a relatively small head.

### 5.3.2.1 Undercushion

In the examples shown in FIGS. 43-77, the seal forming structure 3100 comprises an undercushion 3225. The undercushion 3225 is formed from foam in the illustrated examples but it is to be understood that other materials and structures may also be used to form the undercushion, such as an elastomeric structure, an inflatable undercushion, a gel, a solid semi-rigid material, e.g. silicone or TPE. The undercushion 3225 may be formed for example from a soft foam material that can readily compress and shape to the portions of the face that it engages under the membrane portion 3220. The undercushion 3225 is shaped to extend around the patient's mouth and be positioned proximate to an inferior periphery of the patient's nose in use. The undercushion 3225 may engage the patient's face around the mouth and, in some examples, around the nose periphery. The undercushion 3225 may be air-impermeable. Examples of patient interfaces 3000 made in accordance with the present invention may be referred to as ultra-compact full face masks.

Attached to the undercushion 3225 is the membrane portion 3220 configured to contact the patient's face in use to form a seal around the nose and/or mouth, as will be described below. The undercushion 3225 may hold the membrane portion 3220 in place and in shape to form a seal to the patient's face. In some examples the undercushion 3225 may press the membrane portion 3220 against the user's face around the user's mouth in use. In some examples the undercushion 3225 may support the membrane portion 3220 around the patient's nose so that the membrane portion 3220 engages the patient's nose but the undercushion 3225 remains substantially clear of the patient's nose, e.g. sufficiently to avoid nasal occlusion, save for small amounts of contact that may inevitably occur during use. The undercushion 3225 may be attached to a patient-facing side of the fascia portion 3240.

In the examples shown in FIGS. 43-77, the undercushion 3225 is formed from foam. In some examples the undercushion 3225 may be formed from foam and may comprise a skinned surface. A skinned surface may help with impermeability to air and/or water and with cleanability. In some examples, the undercushion 3225 is formed from a polyurethane foam or a polypropylene foam. In other examples the undercushion 3225 is formed from another suitable foam material. The undercushion 3225 may be formed from open celled foam or closed cell foam, in examples. The undercushion 3225 may be air impermeable, for example due to a skinned surface or an air-impermeable coating, for example.

In the examples shown in FIGS. 43-53, the undercushion 3225 is formed from moulded foam. Moulding the undercushion 3225 may allow for a more complex three-dimensional shape and/or allow for finer details to be provided than if the undercushion 3225 is formed by some other methods, such as cutting from a foam sheet. As described above, the fascia portion 3240 to which the undercushion 3225 is attached may comprise a curved three-dimensional shape in use. In some examples in which the undercushion 3225 is formed from moulded foam, the fascia portion 3240 may be unable to support itself in the curved three-dimensional in use shape (for example, it may not be thermoformed or otherwise structured to take on a particular shape). In such examples the undercushion 3225 may support the fascia portion 3240 in the curved three-dimensional shape. The moulded undercushion 3225 may have sufficient stiffness that the fascia portion 3240, when attached to the undercushion 3225, takes on a curved three-dimensional in use shape. The fascia portion 3240 may be attached to a non-patient-facing side of the undercushion 3225. The non-patient-facing side of the undercushion may comprise a surface corresponding to the curved three-dimensional shape of the fascia portion 3240 in use. The use of a moulded undercushion 3225 which is able to hold the fascia portion 3240 in an in-use shape may avoid the need for a step of forming the fascia portion 3240 into a particular shape, e.g. thermoforming, or avoid the need for added rigidising/shaping components, reducing cost and/or weight of the patient interface 3000.

FIGS. 45 and 47-50 show a moulded foam undercushion 3225. The moulded undercushion 3225 is able to support itself in the shape shown, including having a curvature from one lateral side to the other on both anterior and posterior sides of the undercushion 3225. FIG. 45 shows a fascia portion 3240 which is thermoformed into a three-dimensional shape corresponding to the shape of an anterior side of the undercushion 3225. FIG. 46 shows the two components attached. The fascia portion 3240 in this and other examples may be attached to the undercushion 3225 by any suitable process, including gluing, welding or otherwise bonding etc. In some examples the undercushion 3225 may bonded to the fascia portion 3240 during the moulding process. It is to be understood that while FIG. 45 shows a fascia portion 3240 formed into a curved three-dimensional shape, in other examples it may be formed in a generally flat sheet configuration and may then take the curved shape of the anterior side of the undercushion 3225 when attached to the undercushion 3225.

In the examples shown in FIGS. 54-76, the undercushion 3225 is formed from compression cut foam. Compression cut foam is able to have three-dimensional features but may not have sufficient structural stiffness to support itself in a shape that curves from lateral side to the other. For example, an undercushion 3225 formed from compression cut foam may not be able to be shaped with a curved anterior/non-patient-facing side surface while also comprising curvature on the patient-facing side to match the curvature of the patient's face. In contrast, a compression cut foam undercushion 3225 may be generally flatter and may need another component to hold it in an in-use shape. In such examples the fascia portion 3240 may be thermoformed (or otherwise formed) into a curved three-dimensional shape so as to support itself in the three-dimensional shape in use. An undercushion 3225 formed from compression cut foam may then be attached to the fascia portion 3240, which may support both itself and the undercushion 3225 in predetermined in use shapes. The final shapes of both the fascia portion 3240 and undercushion 3225 may be shapes that generally curve from one lateral side to the other to fit closely to the patient's face in use, to provide for a low-profile patient interface 3000.

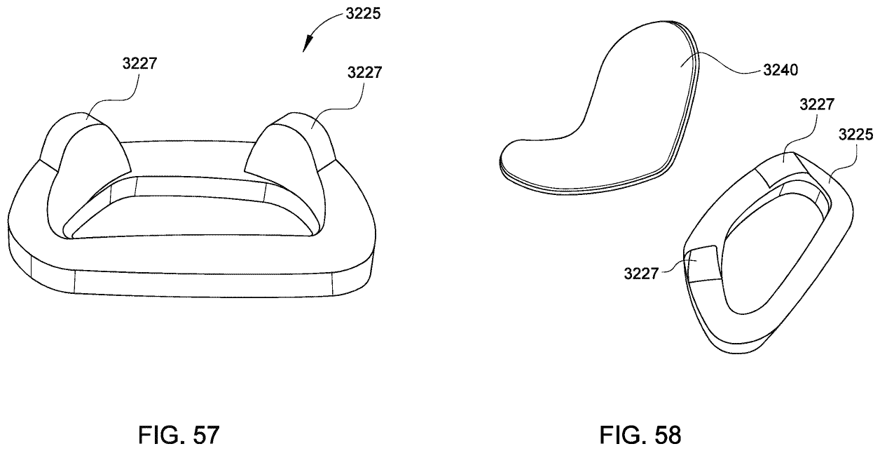

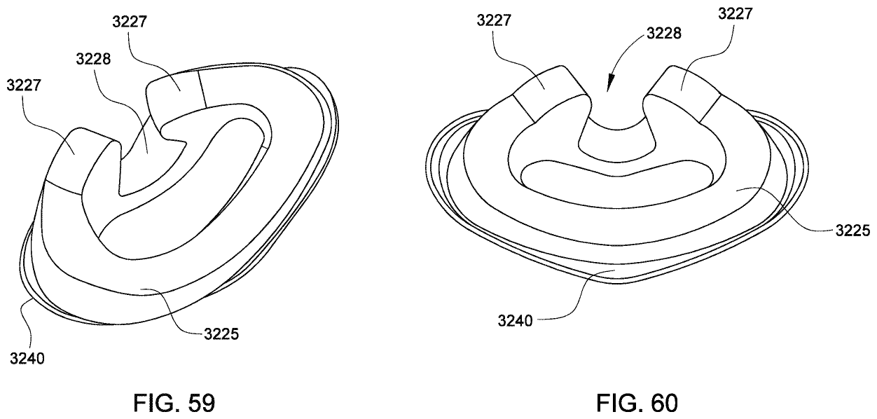

FIGS. 57-62 show views of a fascia portion 3240 and a compression cut foam undercushion 3225. As shown in FIG. 57, the undercushion 3225 has a generally flat configuration with the exception of localised three-dimensional features. For example, an anterior surface of the undercushion 3225 is generally planar. FIG. 58 shows that in comparison to the generally uncurved undercushion 3225, the fascia portion 3240 is shaped to curve from one lateral side to the other lateral side. The fascia portion 3240 has a curved three-dimensional shape, being in substantially the same shape as an in-use shape, for example the shape shown in FIG. 66. FIGS. 59 and 60 show the compression cut undercushion 3225 shown in FIGS. 57 and 58 attached to the fascia portion 3240. As is apparent from FIGS. 59 and 60, after the undercushion 3225 is attached to the fascia portion 3240, the undercushion 3225 is held in a curved shape corresponding to the curvature of the fascia portion 3240. The fascia portion 3240 holds the undercushion 3225 in an in-use shape, which in the illustrated examples is a curved three-dimensional shape.

Similarly, FIGS. 70-71 show an undercushion 3225 according to another example of the present technology formed by compression cutting in a relatively flat shape and FIG. 73 shows the undercushion 3225 attached to a fascia portion 3240, after which it assumes a relatedly more curved shape (in comparison to the relatively flat shape in which it is formed). The fascia portion 3240 is shaped to curve posteriorly in lateral directions away from a medial region of the fascia portion 3240 and the fascia portion 3240 may impart a corresponding shape to the undercushion 3225. In some examples, the fascia portion 3240 may the shape of a hyperbolic paraboloid or parabolic cylinder. As shown in particular in FIG. 73, the undercushion 3225 is more curved at medial locations once attached to the fascia portion 3240 than it is in the flat configuration shown in FIGS. 70 and 71. FIG. 76 shows side-by-side the undercushion 3225 in a formed shape and the fascia portion 3240, which has a posterior side with a greater curvature than formed shape of the undercushion 3225. The fascia portion 3240 may impart a predefined three-dimensional shape to the undercushion 3225. In some examples, the undercushion 3225 may be more curved about either or both of horizontal and vertical axes after attachment to the fascia portion 3240 in comparison to a flatter formed shape in which the undercushion 3225 is formed, e.g. by compression cutting. The fascia portion 3240 may be stiffer than the undercushion 3225 in order to impart a shape to the undercushion 3225. The three-dimensional shape of the undercushion 3225 after attachment to the fascia portion 3240 is a predetermined shape configured to provide for a comfortable, effective and stable seal in use.

In the example shown in FIGS. 70 and 71, the undercushion 3225 may comprise a substantially flat anterior (e.g. non-patient facing) side and a posterior (e.g. patient-facing) side which comprises curved surfaces configured to engage portions of the patient's face in order to effect a seal. The anterior surface of the undercushion 3225 may take on the shape of a posterior-facing surface of the fascia portion 3240, which in some examples may be the shape of a hyperbolic paraboloid or parabolic cylinder or may be a shape described elsewhere herein with reference to a fascia portion 3240 of another example of the present technology.

The undercushion 3225 may comprise curvature on the patient-facing side thereof to form one or more features such as a nasal recess 3228, an inwardly-facing wall 3226 and posterior support portion 3227, which will be described detail below. These are identified in FIGS. 70 and 71 showing the flat configuration of the undercushion 3225 and may each take a final shape and position once the undercushion 3225 is attached to the fascia portion 3240 and deformed, curved and/or bent in the process. The description below of the nasal recess 3228, inwardly-facing wall 3226 and posterior support portion 3227 and any associated features or portions is to be understood to be applicable to the example shown in FIGS. 70-77, unless context requires otherwise.

As can be seen in FIGS. 70, 71, 73 and 76 in particular, the undercushion 3225 comprises a rounded patient-facing surface. This rounded surface may advantageously provide for comfortable engagement with the patient's face when the undercushion 3225 is compressed against the patient's face, at least in comparison to some other shapes which may have sharp corners.

As shown in FIG. 75, the undercushion 3225, is shaped such that when the membrane portion 3220 is attached (to the undercushion 3225 and/or the fascia portion 3240), the membrane portion 3220 has a curvature in the mid-sagittal plane in use (indicated by the broken line) from a superior portion of the cushion module 3150 to an inferior portion which is substantially smooth, e.g. devoid of corners, tight creases or sharp changes in geometry. FIG. 75 also shows a nasal hole 3171 and oral hole 3172. In this example the nasal and oral holes 3171, 3172 are aligned relative to the shape of the undercushion 3225, e.g. in predetermined locations. Both holes are spaced from the undercushion 3225. The membrane portion 3220 at the periphery of each hole 3171, 3172 is not in contact with the undercushion 3225. Advantageously, this allows pressure in the plenum chamber 3200 to act on the membrane portion 3220 to form a pressure-assisted seal. As shown in FIG. 74, the membrane portion 3220 is attached to the undercushion 3225 substantially at and along the outer periphery of the undercushion 3220.

### 5.3.2.1.1 Nasal Recess

In some forms of the technology, such as are shown in particular in FIGS. 45-52, 59-65 and 70-77 for example, the undercushion 3225 may comprise a nasal portion configured to be positioned proximate the inferior periphery of the patient's nose in use. The undercushion 3225 may comprises a nasal recess 3228 in the nasal portion, and the nasal recess 3228 may be shaped to receive the patient's nose in use. The nasal recess 3228 in some forms of the technology may be shaped to receive the patient's nose without engaging the patient's nose or at least without applying an occluding force to the patient's nose. The nasal recess 3228 may be configured to at least partially surround the inferior periphery of the patient's nose in use. In some examples the nasal recess 3228 provides clearance between the undercushion 3225 and the inferior periphery of the patient's nose (e.g. inferior portions of the nasal alae and pronasale). The nasal recess 3228 may be configured to prevent the seal-forming structure 3100 from occluding the patient's nose, for example when the headgear is tightened. The membrane portion 3220 may be supported by the undercushion 3225 at a periphery of the nasal recess 3228. For example, the membrane portion 3220 may be attached to the undercushion 3225 at or proximate a periphery of the nasal recess 3228. The membrane portion 3220 may be in contact with and/or supported by the undercushion 3225 at the periphery of the nasal recess 3228 but may be free of contact with the undercushion 3225, e.g. unable to contact the undercushion 3225 in use, at a medial region inside of the periphery of the nasal recess 3228, such as a medial region inferior to and aligned horizontally with the patient's nose. The membrane portion 3220 may be unable to contact the undercushion 3225 in a region of the membrane portion 3220 contacting inferiorly-facing surfaces of the patient's nose.

In some examples the nasal recess 3228 may space the undercushion 3225 from the patient's nose at least when the patient initially dons the patient interface 3000 prior to sleeping, so as to not press the membrane portion 3220 against the patient's nose, or at least not press the membrane portion 3220 against the nasal ala. The nasal recess 3228 may prevent the undercushion 3225 from engaging the patient's nose or at least prevent the undercushion 3225 from occluding the patients nose (e.g. by not engaging it with sufficient force to close or partially close the nostrils). The nasal recess 3228 may prevent inadvertent occlusion of the patient's nose which may otherwise occur if the undercushion 3225 was shaped to engage the nose together with the membrane portion 3220 (e.g. by pressing the membrane portion 3220 against the nasal alae). It is to be understood that on some patients with large noses the undercushion 3225 may nevertheless have a small amount of engagement with portions of a user's nose, but due to the nasal recess 3228 may not occlude the user's nose. For example, the nasal recess 3228 may be configured to avoid engaging both lateral sides of the patient's nose simultaneously. In some examples, when the patient's nose is centred within the nasal recess 3228, the nasal recess 3228 substantially does not engage either lateral side of the patient's nose. In some examples the nasal recess 3228 may be structured to provide substantially no medially-directed forces on the patient's nasal alae in use. In some forms, the nasal recess 3228 is structured and arranged such that the undercushion 3225 applies substantially no force on the patient's nose in use. In some forms, the nasal recess 3228 is structure and arranged such that there may be some contact with the patient's nose, but without sufficient force to occlude the patient's nostrils and which may be with minimal force in order to be comfortable for long periods.

The undercushion 3225 and fascia portion 3240 may be constructed such that in use they are positioned very close to the patient's face in use to allow for engagement between the undercushion 3225 with the patient's cheeks and mouth region. The nasal recess 3228 may allow for the undercushion 3225 to be urged against the patient's cheeks and mouth region without also excessively urging the patient's nasal alae closed, which could occlude the patient's nasal airways.

The nasal recess 3228 (e.g. the portion of the undercushion 3225 forming the nasal recess 3228) in the examples shown in FIGS. 43-77 comprise a shape corresponding to the inferior periphery of the nose (e.g. the inferior shape of the pronasale portion and nasal alae). The corresponding shape, combined with only a small spacing between the nasal recess 3228 and patient's nose in use may provide for a low-profile patient interface 3000.

In some forms, such as are shown in FIGS. 49-52, 63 64, and 70-77, the nasal recess 3228 may comprise an inwardly-facing wall 3226. The inwardly-facing wall 3226 may face towards the patient's nose and may also be described as a nasal-facing wall. The inwardly-facing wall 3226 may face partially inwardly and partially superiorly. For example, the inwardly-facing wall 3226 may be structured to face and at least partially surround the inferior periphery of the patient's nose in use. The inwardly-facing wall 3226 in these examples may be one or more of substantially chamfered, frustoconical, bowl-shaped, frustospherical, parabolic, among other possible shapes. The inwardly-facing wall 3226 may be concave, in some examples concave in two orthogonal planes. For example, the inwardly-facing wall 3226 may comprise a concave shape when viewed in a vertical cross-section (e.g. a cross-section parallel to the sagittal or coronal plane) and also when viewed in a horizontal cross-section (e.g. a cross-section parallel to the Frankfort horizontal). In other examples the inwardly-facing wall 3226 may not be concave in a vertical cross section, for example if the inwardly-facing wall 3226 is chamfered or otherwise appears as a straight line in vertical cross section. In some examples the inwardly-facing wall 3226 may appear as a straight line in vertical cross section in one location (e.g. proximate the pronasale) but may appear as a curved line in vertical cross section in another location (e.g. on a lateral side of the patient's nasal ala), or vice versa.

The inwardly-facing wall 3226 may comprise a pair of lateral portions on either lateral side of the nasal recess 3228. The lateral portions may face medially and may be positioned in use on either lateral side of the user's nose in use. The inwardly-facing wall 3226 may be concave. The lateral portions of the inwardly-facing wall 3226 may face partially medially and partially superiorly. The inwardly-facing wall 3226 may comprise the same or a similar shape at an anterior portion of the inwardly-facing wall 3226, for example at a location proximate the patient's pronasale. The anterior portion of the inwardly-facing wall 3226 may face posteriorly and may be positioned anterior and/or inferior to the patient's pronasale. The anterior portion of the inwardly-facing wall 3226 may face partially posteriorly and partially superiorly. Similarly to the lateral portions of the inwardly-facing wall 3226, the anterior portion of the inwardly-facing wall 3226 may be concave.

The inwardly-facing walls 3226 in the examples shown in FIGS. 49-52, 63-64 and 70-77 are concave in a horizontal plane. The concavity in a horizontal plane results in the nasal recess 3228 surrounding the inferior periphery of the patient's nose. The shape of the nasal recess 3228 and the inwardly-facing wall 3226 thereof in a horizontal plane may be semi-circular or may be a non-circular concave shape, such as a shape closer to a triangle with a rounded anterior corner, lateral sides and no base. In some examples, the nasal recess 3228 may generally have a shape in a horizontal plane that follows a general shape of the patient's nose, for example the inferior periphery of the patient's nose. In some forms the inwardly-facing wall 3226 comprises, viewed in a horizontal cross section, a concave anterior portion facing posteriorly and two lateral portions. The lateral portions may face predominantly medially and to a lesser extent posteriorly. Such a shape may more closely follow the shape of a narrow and long nose. In some examples the undercushion 3225 (or a cushion module comprising the undercushion 3225) are provided in a range of sizes and/or shapes, the shape of the nasal recess 3228 and inwardly-facing wall 3226 thereof differing the range of cushion module options so that each patient can select the cushion module having the best fitting undercushion 3225 for their nose and face.

The nasal recess 3228 in some examples surrounds substantially all of the laterally and anteriorly facing portions of the inferior periphery of the patient's nose in use. The inwardly-facing wall 3226 may extend from at or proximate one of the patient's cheeks around the patient's nose to at or proximate the other of the patient's cheeks.

With particular reference to FIGS. 46-51, 57-64 and 70-77, each of the undercushions 3225 in these particular examples further comprises a pair of posterior support portions 3227 each positioned on a respective lateral side of the nasal recess 3228 and configured to engage the patient's face medially of and proximate to the nasolabial sulci of the patient's face. These posterior support portions 3227 may engage the patient's face at locations inferior to the nasal alae on either lateral side of the patient's lip superior, or may engage the patient's face at locations aligned vertically with the patient's nasal alae between the nasal alae and the nasolabial sulci, for example. In some examples, the posterior support portions 3227 may engage the patient's face in an area that extends vertically from alongside the nasal alae to alongside the patient's lip superior. The posterior support portions 3227 may engage the face to support the patient interface 3000 in position and may also advantageously encourage the membrane portion 3220 to form a good seal proximate the inferior corners of the patient's nose, which may be more difficult to seal against on a wide range of patients in comparison to other places such as the cheeks. As shown in FIGS. 49, 50, 51 and 73 for example, the inwardly-facing wall 3226 may extend from a first one of the posterior support portions 3227 around the patient's nose to the other one of the posterior support portions 3227.

The posterior support portions 3227 in the examples shown in FIGS. 49, 50, 51 and 73 also each include a region of the inwardly-facing wall 3226, although this may be a relatively small region in comparison to the overall size of the inwardly-facing wall 3226. The posterior support portions 3227 are in these examples each shaped to protrude at least partially medially into concavities formed on the patient's face on either lateral side of the nasal alae, for example between the nasal alae and adjacent portions of the patient's face. For example, a posterior-facing surface or portion of each posterior support portion 3227 may engage the patient's face in a region between the nasolabial sulcus and nasal ala. At the same time, a portion of the inwardly facing surface 3226 forming part of or located proximate to each of the posterior support portions 3227 may engage a respective one of the patient's nasal alae. The posterior support portions 3227 may “dig in” to the concavities on either side of the patient's nose to assist in achieving a good seal in those regions. The posterior support portions 3227 may engage only a rearmost portion of the nasal ala. The posterior support portions 3227 may contact the nasal ala for the purpose of encouraging sealing of the concavity at either lateral side of the inferior periphery of the patient's nose. The undercushion 3225 is still configured to avoid exerting medially-directed forces on the patient's nose which may tend to occlude the nasal airways.

As shown in each of FIGS. 49, 50, 51 and 73 for example, the posterior support portions 3227 are shaped to correspond closely to the geometry of the surfaces of a patient's face at and proximate the alar crest point, e.g. where each of the nasal alae meet the patient's cheeks. In these examples the posterior support portions 3227 each comprises a posterior-facing surface 3227a and a medially-facing surface 3227b. It is to be understood that the medially-facing surface 3227b may face partially anteriorly, as well as medially. Similarly, the posterior-facing surface 3227a may face partially medially, as well as posteriorly. The posterior-facing surface 3227a of each posterior support portion 3227 for example may be convex to complement the concavity in the patient's face in the region between the nasolabial sulcus and the inferior periphery of the patient's nose. The medially-facing surface 3227b of each posterior support portion 3227 may for example be concave to match the convex shape of each of the patient's nasal alae.

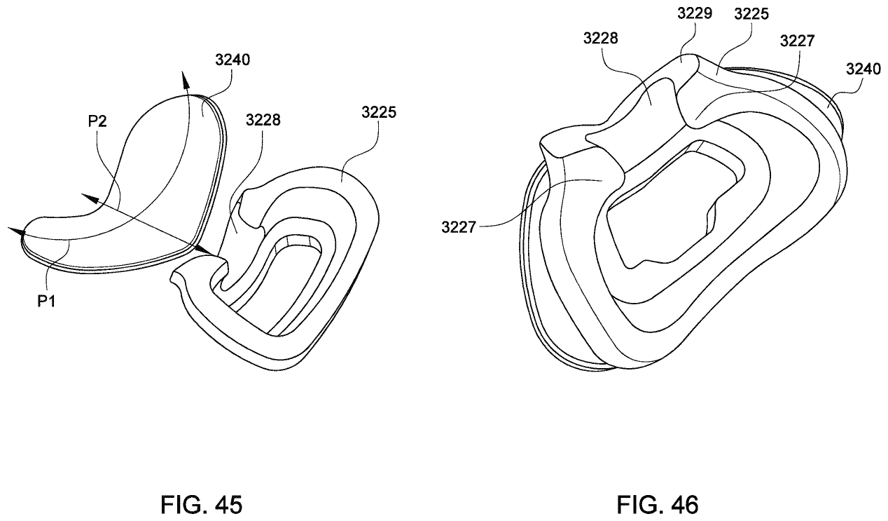

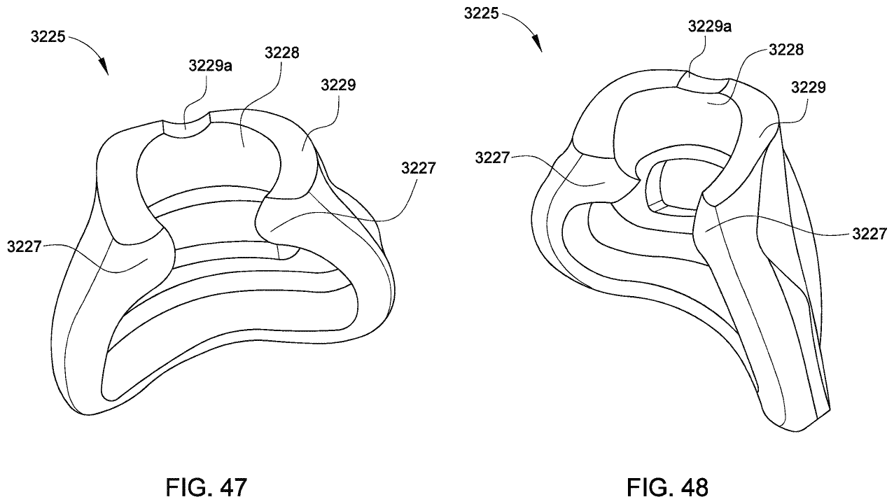

In the examples shown in FIGS. 43-77 the membrane portion 3220 is attached to the undercushion 3225 around the periphery of the nasal recess 3228. The undercushion 3225 in these examples comprises a superiorly-facing surface 3229 located adjacent to and provided around the periphery of the nasal recess 3228, the membrane portion 3220 being attached to the superiorly-facing surface 3229 of the undercushion 3225. The membrane portion 3220 may be glued, bonded, welded or otherwise attached to the superiorly-facing surface 3229. The superiorly-facing surface 3229 may be flat, in some examples. In other examples the superiorly-facing surface 3229 may have a small curvature in cross section but not to an extent that the membrane portion 3220 cannot robustly attach to the superiorly-facing surface 3229. In other examples the membrane portion 3220 is attached to an attachment surface of the nasal portion which may or may not be superiorly-facing. This portion defining the nasal recess 3228 may have a consistent wall cross section around the nasal recess 3228 to avoid weak spots in some examples. That said, in some examples the nasal portion of the undercushion 3225 may comprise a medial recess 3229a in the superiorly-facing surface 3229, as shown most clearly in FIGS. 47, 48, 49 and 50 for example. The medial recess 3229a may be configured to provide clearance for the pronasale and/or reduce pressure on the pronasale in the event there is inadvertent engagement between the undercushion 3225 and the pronasale. While the nasal recess 3228 and medial recess 3229a are intended to provide clearance for the nose, and pronasale thereof, respectively, it is to be understood that for some patients there may nevertheless be some engagement between the undercushion 3225 and nose in one or more locations. However, the nasal recess 3228 and medial recess 3229a are still to be understood to be configured (e.g. structured, shaped, arranged and/or positioned) to provide clearance as they are generally shaped to avoid contact with the nose and pronasale, respectively, and result in less contact than would otherwise occur.

With particular reference to FIGS. 51 and 52, in some forms of the present technology the nasal portion of the undercushion 3225 comprises a medial peripheral portion 3231 partially defining the periphery of the nasal recess 3228. The medial peripheral portion 3231 are structured to support the membrane portion 3220 at a medial position proximate the patient's pronasale. The nasal portion may further comprise a pair of lateral peripheral portions 3232 also partially defining the periphery of the nasal recess 3228. The lateral peripheral portions 3232 are located on respective lateral sides of the medial peripheral portion 3231. The lateral peripheral portions 3232 are structured to support the membrane portion 3220 superiorly of the patient's nasal alae on respective lateral sides of the patient's nose (as shown in FIG. 52 for example). The lateral peripheral portions 3232 may be structured to support the membrane portion 3220 superiorly of the medial position at which the medial peripheral portion 3231 supports the membrane portion 3220.

As shown in FIG. 51, the lateral peripheral portions 3232 may curve superiorly along the periphery of the nasal recess away from the medial peripheral portion 3231 to respective superior-most points of each lateral peripheral portion 3232. Posteriorly of the superior-most points, the lateral peripheral portions 3232 curve inferiorly along the periphery of the nasal recess 3228 away from the respective superior-most points in a posterior direction. Medially, the medial peripheral portion 3231 curves superiorly on either lateral side thereof into the lateral peripheral portions 3232. As shown in FIG. 51, each lateral peripheral portion 3232 comprises a dome shape at the respective superior-most point. The lateral peripheral portions 3232 may be convex in two orthogonal axes at the superior-most points. Also as shown in FIG. 51, the medial peripheral portion 3231 comprises a saddle shape. The medial peripheral portion 3231 may be convex in an anterior-posterior axis but may be concave in the medial-lateral directions along the periphery of the nasal recess.

With reference to FIG. 52, the lateral peripheral portions 3232 are advantageously positioned high (e.g. quite superiorly) with respect to the inferior periphery of the patient's nose and with respect to the patient's nasal alae. This provides for a large presentation angle P, indicated in FIG. 52. The presentation angle P is an angle of each side of the membrane portion 3220 in use. A large presentation angle P encourages the membrane portion 3220 to wrap upwardly/superiorly around the nasal ala on each lateral side of the user's nose. The tendency to do this may help provide for a stable/robust seal in use and may facilitate the patient interface 3000 being able to accommodate a wide range of patient nose shapes and sizes.

Additionally, the high sides of the nasal portion and deep positioning of the patient's nose in use may provide for easier patient setup/fitment since the patient interface 3000 may be more accommodating to a range of patient anatomy. Furthermore, since the patient's nose will be effectively suspended within the nasal recess 3228, upon application of therapy pressure, headgear tension and some jiggling, the patient's nose may tend to become positioned correctly when the membrane portion 3220 securely engages and partially wraps around the inferior periphery of their nose, due to the patient's nose naturally tending to occupy a low, suspended, position within the nasal recess 3228.

As described in more detail elsewhere, the patient interface 3000 in the examples shown in FIGS. 43-77 comprises a fascia portion 3240 at least partially defining the plenum chamber 3200, the undercushion 3225 being attached to a patient-facing side of the fascia portion 3240. In the particular examples shown in FIGS. 51 and 52 and 70-77, each of the lateral peripheral portions 3232 extends superiorly of a superior edge/boundary of the fascia portion 3240. The lateral peripheral portions 3232 are free from attachment to the fascia portion 3240 above/superior of the fascia portion 3240. The lack of attachment of some or all of each lateral peripheral portion 3232 to the fascia portion 3240 may advantageously facilitate flexing/bending of the lateral peripheral portions 3232, which is described below.

In some examples, such as in the example shown in FIG. 72, the portions of the undercushion 3225 located adjacent to the nasal ala, e.g. lateral peripheral portions 3232, are the only portions of the undercushion 3225 that project past a peripheral edge of the fascia portion 3240. These regions may effectively be less stiff than other regions due to the lack of support on the non-patient facing side.

### 5.3.2.1.2 Flexing

While much of the undercushion 3225 that engages directly around the nose and mouth is configured to deform by compression in use, one or more portions of the undercushion 3225 may be configured to deform in another manner. In some forms of the technology, the undercushion 3225 may be configured to flex in response to force applied to the undercushion 3225 in use.

In particular, in some forms of the present technology, the patient interface 3000 comprises a seal-forming structure 3100 comprising an undercushion 3225 and a membrane portion 3220 connected to the undercushion 3225. The undercushion 3225 may be structured to bend in one or more locations when the patient dons the patient interface. FIGS. 68 and 69 respectively show cross section views of such an undercushion 3225 through a lateral peripheral portion 3232 (in FIG. 68) and through a lip inferior portion (in FIG. 69).

The undercushion 3225 may be configured to bend in cross section. In some examples the undercushion 3225 comprises a portion of the undercushion proximate a periphery of the undercushion 3225 that bends. This may be distinguished from bending of the undercushion 3225 as a whole. In some examples, the undercushion 3225 is structured to bend in the manner of a first portion of a cross section through the undercushion 3225 bending with respect to a second portion of the cross section through the undercushion 3225. For example, with reference to FIG. 68, the superior portion of the undercushion 3225 proximate the nasal alae may bend with respect to the inferior portion connected to the fascia portion 3240. In some examples, the first portion of the cross section through the undercushion 3225 forms a patient-facing side of the undercushion 3225 and urges the membrane portion 3220 of the seal-forming structure 3100 against the patient's face in use, and the second portion of the cross section through the undercushion 3225 forms a non-patient facing side of the undercushion 3225 in use. For example, with reference to FIG. 69, the left side of the undercushion 3225, when viewed in the cross section shown in FIG. 69, urges the membrane portion 3220 against the patient's face, and may form a first portion that bends with respect to a second portion of the undercushion 3225 which doesn't engage the user's face (e.g. the right hand side of the cross section in FIG. 69.

In examples, the undercushion 3225 may be structured to bend when the patient dons the patient interface 3000 in any one or more of the following locations: proximate the user's nose, proximate the user's mouth, proximate the user's lip inferior and/or proximate the user's cheeks.

In some patient interfaces 3000 according to examples of the present technology comprising an undercushion 3225 having a nasal portion forming a nasal recess 3228, such as the type of undercushion 3225 of the patient interface 3000 shown in FIG. 51, the undercushion 3225 may comprise lateral peripheral portions 3232, as are described elsewhere herein. In some forms of the present technology, the lateral peripheral portions 3232 of the undercushion 3225 are flexible and structured to deform medially towards the patient's nose in use. In some examples, the lateral peripheral portions 3232 are structured to bend medially towards the patient's nose in use. In some examples, such as the examples shown in FIGS. 51, 52 and 68, the lateral peripheral portions 3232 are supported at inferior ends and are free at superior ends (other than their connections to the membrane portion 3220) and so function in the manner of cantilever beams. In use the patient's nose exerts a force inferiorly on the membrane portion 3220 which will tend to pull the superior (free) ends of the lateral peripheral portions 3232 medially. This may advantageously facilitate the membrane portion 3220 wrapping around the lateral edges of the inferior periphery and/or nasal alae of the patient's nose, which may provide for a stable seal on a wide range of patient nose shapes and sizes and/or may facilitate easy setup.

With reference to examples depicted by way of partial cross sections in FIGS. 68 and 69, in some forms, the undercushion 3225 may comprise one or more hinge regions 3234 at which the undercushion 3225 is structured to bend when the patient dons the patient interface 3000. In some forms, each of the hinge regions 3234 may be formed by a channel 3233 formed in the undercushion 3225 forming a region of reduced thickness, such as the channels depicted in FIGS. 68 and 69.

FIG. 69 shows an oral channel 3233 forming a hinge region 3234 in a lip inferior region of the undercushion 3225. This may facilitate bending in the lip inferior region of the undercushion 3225 to supplement the compressibility of the material forming the undercushion 3225. The oral channel 3233 may additionally or alternatively be provided in cheek regions of the undercushion 3225. In some examples, the oral channel 3233 extends along a first cheek region, along the lip inferior region and along a second cheek region.

FIG. 68 shows a nasal channel 3233 provided in a nasal recess 3228 of an undercushion 3225. The nasal channel 3233 may extend laterally around the nasal recess, e.g. following an inwardly facing wall 3226 below the inwardly-facing wall 3226. The undercushion 3225 may also comprise a pair of posterior support portions 3227 each positioned on a respective lateral side of the nasal recess 3228 and configured to engage the patient's face medially of and proximate to the nasolabial sulci of the patient's face. The nasal channel 3233 may be provided proximate the posterior support portions 3227, for example. In some forms a nasal channel 3233 and an oral channel 3233 meet at respective lateral sides of the undercushion 3225 to form a single contiguous channel 3233.

### 5.3.2.1.3 Undercushion Material

In examples the undercushion 3225 formed from a foam material is made from a polyurethane, either a thermoplastic or thermoset polyurethane, or from a thermoplastic elastomer (e.g. a soft TPE having similar behaviour to a foam). Foam having the following properties may be suitable:
Force Deflection: 95+/−20N @40% (Test spec: AS 2282.8 Method A)
Density: 54.5+/−2.5 kg/m3 (Test spec: AS 2282.3)
Air Permeability: <1.5 l/min @ 20 cm H2O.

In some examples, foam having a lower density than the above may be used to form the undercushion 3225. In some examples the undercushion 3225 may be formed from a low density foam but may have a sufficiently thick base or body to it that it is able to sufficiently support the membrane 3221 and support itself, especially in the nose region. In some forms the undercushion 3225 may be formed from a low density foam but may have a sufficiently thick skin. The undercushion 3225 may be provided with a sufficiently thick skin to provide for sufficient air-tightness, water-tightness, stiffness and/or comfort.

In other examples any of a polypropylene foam, polyethylene foam, a silicone foam or an EVA foam may be used, among other examples.

In some examples a foam undercushion 3225 may be formed by moulding, e.g. injection moulding.

In examples the foam may be substantially water impermeable so that the mask can dry adequately before use at night when washed earlier in the day.

In examples the undercushion material is soft enough to cause substantially no discomfort when engaging the face, but is firm enough to retain the fascia portion 3240 and hold the membrane portion 3220 in a sealed manner when the patient interface is worn.

In examples the foam from which the undercushion 3225 is made is substantially air impermeable, for example, the foam may be a skinned foam.

### 5.3.2.2 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.2.3 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.2.4 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.2.5 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.2.6 Forehead Region

In one form, the seal-forming structure that forms a seal in use on a forehead region of the patient's face. In such a form, the plenum chamber may cover the eyes in use.

### 5.3.2.7 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.2.8 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3Z, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.2.9 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to check regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.2.10 Membrane Portion

As discussed above, in examples the patient interface 3000 may comprise a membrane portion 3220 which engages the patient's face. In use, the undercushion 3225 supports the membrane portion 3220. The membrane portion 3220 may be at least partially formed from a textile material, from an elastomer such as silicone or TPE, or from another suitable material, in various examples. The membrane portion 3220 may be air-impermeable. In particular, in one form, such as shown in the examples of FIGS. 43-77, the seal-forming structure includes a textile membrane portion 3220 which functions as a pressure assisted sealing mechanism. In use, the textile membrane portion 3220 can readily respond to a system positive pressure in the interior of the plenum chamber 3200 acting on its interior side to urge it into tight sealing engagement with the face. The pressure assisted mechanism may act in conjunction with elastic tension in the positioning and stabilising structure 3300. In other examples the membrane portion 3220 may be formed from silicone, such as a silicone membrane having a thickness of 0.2-0.4 mm, or 0.25-0.3 mm, for example.

### 5.3.3 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 Gpa or lower, for example foam. In some forms of the technology the plenum chamber 3200 may be made from a material having Young's modulus of 0.1 Gpa or lower, for example rubber. In other forms of the technology the plenum chamber 3200 may be made from a material having a Young's modulus of 0.7 Mpa or less, for example between 0.7 Mpa and 0.3 Mpa. An example of such a material is silicone.

### 5.3.3.1 Multiple Openings

As shown in FIGS. 7A and 7B, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear—described below) or a “tube-down” configuration (e.g., using a single conduit in front of the patient's face).

### 5.3.3.1.1 Nose and Mouth Mask

As shown in FIG. 7A, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 7A). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 5.3.3.1.2 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 7B, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 7B). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

5.3.3.2 Plenum Chamber with Fascia Portion

In the example shown in FIGS. 43-66, the plenum chamber 3200 is partially formed by the seal forming structure 3100 and partially formed by the fascia portion 3240. The plenum chamber inlet port 3202 in this example is a single opening provided in the fascia portion 3240. In particular, a short tube 3610 is connected to the fascia portion 3240 and fluidly connected to the plenum chamber inlet port 3202 to convey a flow of air to the plenum chamber 3200. The patient interface 3000 comprises a connector 3620 connecting the short tube 3610 to the fascia portion 3240 in these examples. The connector 3620, as shown in FIGS. 53, 54 and 66, is an elbow in these examples. In other examples it may be another type of connector, such as a straight connector. The connector 3620 may swivel with respect to the plenum chamber 3200 in some examples. At a distal end of the short tube 3610 the patient interface 3000 may comprise a connection port 3600 which may be connected to an air circuit 4170 connected to an RPT device 4000.

As will be described below, in some examples, the fascia portion 3240 may at least partially form the plenum chamber 3200, for example together with the undercushion 3225 and/or the membrane portion 3220. In other examples the undercushion 3225 and the membrane portion 3220 may form substantially all of the plenum chamber 3200, for example if the undercushion 3225 does not include any openings on an anterior side thereof other than for a connection to a short tube 3610 or other connector to an air circuit 4170. In such an example, the fascia portion 3240 may form a structural component, providing structural stiffness to the cushion module or holding the undercushion 3225 in a predetermined shape.

### 5.3.3.2.1 Fascia Portion

As described above, the patient interfaces 3000 shown in FIGS. 43-66 each comprise a fascia portion 3240, which may partially form the plenum chamber 3200. The fascia portion 3240 may form an anterior side of the patient interface 3000 or at least a cushion module thereof. The fascia portion 3240 may comprise a patient-facing surface, e.g. on a patient-facing side, and the undercushion 3225 may be attached to the patient-facing surface of the fascia portion 3240. The membrane portion 3220 may substantially cover a patient-facing side of the undercushion 3225 and may be attached to the undercushion 3225 and/or the fascia portion 3240.

The fascia portion 3240 may be partially formed from a foam and/or textile material. In examples shown in FIGS. 43-66, the fascia portion 3240 is formed from foam covered with a textile material/textile layer on a non-patient-facing surface thereof. The textile material on the non-patient-facing/anterior-facing surface may advantageously provide for a comfortable feel when the patient interface 3000 is handled and an appearance consistent with bedclothes and comfort, which may be more likely to result in patient compliance with therapy than if the patient interface 3000 were to look more like purely a medical device. In some examples, the fascia portion 3240 further comprises a textile layer on the patient-facing surface. In some examples, the foam material partially forming the fascia portion 3240 may be a polyurethane foam. In some examples the foam material may be a closed cell foam, which may advantageously provide air impermeability. In some examples, the textile material forming on both sides of the fascia portion 3240 may be formed from nylon or polyester and may be woven, for example knitted. In some examples, the fascia portion 3240 may comprise an air impermeable film provided between the textile layer(s) and the foam layer, which may provide air and/or moisture impermeability and may allow for the use of an open cell foam in the fascia portion 3240.

In some examples the fascia portion 3240 is formed from foam with no textile. In such an example the foam may be air impermeable by nature, e.g. it may be a closed cell foam. In other examples it may be coated with an air-impermeable layer (e.g. a silicone, polyurethane or other air-impermeable film/sheet). A foam material is not essential in the fascia portion 3240 and in some examples may be replaced by another material, such as a thick synthetic material able to be shaped by thermoforming. In some examples the fascia portion 3240 is formed from a textile material (e.g. with no foam), which may be, for example, 2-5 mm in thickness. The textile material may be covered/coated with one or more air-impermeable layers to provide for air-impermeability. In some examples the fascia portion 3240 may comprise a synthetic felt material. In some examples in which the undercushion 3225 and membrane portion 3220 define the plenum chamber 3200 and the fascia portion 3240 does not, the fascia portion 3240 may not be made air-impermeable.

The fascia portion 3240 may comprise a curved three-dimensional shape in use. In the illustrated examples, the fascia portion 3240 is thermoformed into the curved three-dimensional shape so as to support itself in the curved three-dimensional shape. The fascia portion 3240 may be semi-rigid to provide shape and/or structure to the cushion module of the patient interface 3000. In some examples the fascia portion 3240 is structured to support the undercushion 3225. As described elsewhere herein, the undercushion 3225 may be formed from foam and may be unable to support itself in an in-use shape, for example if the undercushion 3225 is formed from compression cut foam. However, in some examples, the fascia portion 3240 is able to support itself in an in-use shape and also support the undercushion 3225 in an in-use shape. In other examples, the fascia portion 3240 is unable to support itself in a curved three-dimensional shape. In such examples the undercushion 3225 may be structured to support itself in a predetermined shape and be able to support the fascia portion 3240 in a curved three-dimensional shape corresponding to an in-use shape. For example, the undercushion 3225 may be formed from moulded foam and may support the fascia portion 3240 in the curved three-dimensional shape. In further examples, neither the fascia portion 3240 nor the undercushion 3225 are able to fully support themselves in predetermined shapes but when attached to each other, e.g. when bonded together or otherwise attached, they may each take on a predetermined shape. The assembly may support itself in a predetermined shape. For example, each of the fascia portion 3240 and the undercushion 3225 may support the other in the predetermined shape once attached. In various examples, each of the fascia portion 3240 and the undercushion 3225 is either unable to support itself (e.g. completely floppy) or only able to partially support itself (e.g. a bit wobbly) prior to attachment to the other.

In some examples the fascia portion 3240 comprises a non-zero negative first principal curvature and a second principal curvature which is less than the first principal curvature. In examples, the fascia portion 3240 is curved such that it has a non-zero negative first principal curvature P1 and a substantially zero second principal curvature P2, as shown in FIG. 45.

In examples, the patient interface 3000 is configured such that the second principal curvature P2 is substantially parallel, in use, to a mid-sagittal plane of the patient and may lie in the mid-sagittal plane in use.

In some examples, the fascia portion 3240 is formed substantially flat but is held in the curved configuration by the undercushion 3225. In other examples the fascia portion 3240 may be inherently curved, for example it may be thermoformed, moulded or otherwise manufactured with an inherent curvature. In some examples the fascia portion 3240 may be sufficiently flexible that despite being held in a curved configuration by an undercushion 3225, the fascia portion 3240 substantially does not deform the undercushion 3225 (e.g. by reaction forces). In some examples the undercushion 3225 holds the fascia portion 3240 in a curved shape across the user's face in the lateral-medial directions and allows for flexing of this curved shape, yet substantially prevents the cross section of the fascia portion 3240 in the sagittal plane from flexing. In some examples, the fascia portion 3240 is shaped as a hyperbolic paraboloid or parabolic cylinder. The junction of the undercushion 3225 and/or the membrane portion 3220 with the fascia portion 3240 may form a path lying on a hyperbolic paraboloid or parabolic cylinder.

The patient interface 3000 may comprise at least one pair of headgear connectors connected to the fascia portion 3240. The headgear connectors may be configured to connect to headgear straps of a positioning and stabilising structure 3300 of the patient interface 3000. As shown in FIGS. 53, 54 and 66 in particular, in some forms of the technology the fascia portion 3240 is provided with a pair of superior headgear connector portions 3310 and a pair of inferior headgear connector portions 3320. In examples, the fascia portion 3240 is also provided with an opening for connection (for example releasable connection) to an air delivery tube, in use. In examples the fascia portion 3240 is provided with at least one vent 3400 for gas washout (e.g. to provide a continuous flow of gas from the plenum chamber 3200 to ambient throughout the patient's respiratory cycle in use). In the examples shown in FIGS. 43-66, the frame 3240 comprises a connector 3620 providing a connection to a short tube 3610 having a connection port 3600 at the end thereof. The connector 3620 may be in the form of an elbow and/or may be configured to swivel with respect to the frame 3240. The vent 3400 in this example is provided to the connector 3620. The vent 3400 may be formed by a plurality of holes. As depicted in FIGS. 53, 54 and 66 (only one side most visible), the superior headgear connector portions 3310 are connected to superior headgear straps 3311 and the inferior headgear connector portions 3320 are connected to inferior headgear straps 3321.

In some examples in which the fascia portion 3240 is relatively flexible, and has a substantially zero principal curvature substantially aligned with a sagittal plane, the fascia portion 3240 may flex relatively easily such that the magnitude of the first principal curvature P1 can be varied when the patient interface 3000 is donned. For example, the magnitude of the first principal curvature may be relatively large (e.g. the frame 3240 may be more curved) when the patient interface 3000 is donned by a person having a relatively narrow face, but may be smaller (e.g. the frame 3240 may be relatively “flatter”) when the patient interface 3000 is donned by a person having a relatively broad face. The fascia portion 3240 in such examples is configured to flex such that the first principal curvature has a larger magnitude when donned by a patient with a relatively narrow face than when donned by a patient having a relatively wider face. In this way, the patient interface 3000 may be adaptable to fit patients having a range of different size faces.

In examples in which there is no preformed curvature in the fascia portion 3240 in the sagittal plane (e.g. the principal curvature P2 described above), the fascia portion 3240 may be configured to flex less readily in the sagittal plane (e.g. changing the second principal curvature P2) than in a horizontal plane (e.g. changing the first principal curvature P1). Furthermore, the fascia portion 3240 may be shorter in the superior-inferior directions than in the medial-lateral directions, which further makes the fascia portion 3240 less likely to flex in the sagittal plane. In some examples the fascia portion 3240 may comprise a small curvature in the sagittal plane (e.g. the principle curvature P2), either by being formed with a small curvature or by being held in a curved shape by the undercushion 3225.

FIG. 67 shows that the lateral sides of the fascia portion 3240 may be angled with respect to each other at an angle A of 90 degrees, in one example. In other examples the lateral sides of the fascia portion 3240 may form an included angle of between 70 degrees and 120 degrees, between 75 degrees and 110 degrees, between 80 and 100 degrees or between 85 and 95 degrees, by way of example only. Additionally, an medial portion of the fascia portion may be curved with a radius of curvature D of substantially 30 mm, in one example. In other examples the radius D may be within the range of 20-40 mm or 25-35 mm.

### 5.3.4 Positioning and Stabilising Structure

The seal-forming structure 3100 of the patient interface 3000 of the present technology may be held in sealing position in use by a positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIGS. 3A and 3A-1.

In one form the positioning and stabilising structure 3300 provides a retention force at least sufficient to overcome the effect of the positive pressure in the plenum chamber 3200 to lift off the face (i.e., Fₚₗₑₙᵤₘ).

In one form the positioning and stabilising structure 3300 provides a retention force to overcome the effect of the gravitational force on the patient interface 3000.

With continued reference to FIG. 3A-1, the positioning and stabilising structure 3300 provides a force F_{PSS} that assists in maintaining the plenum chamber 3200 in the sealing position on the patient's face. The positioning and stabilising force F_{PSS} may be the resultant force from the various forces of the different elements of the positioning and stabilising structure 3300. For example, headgear straps may individually provide a strap force Fₛₜᵣₐₚ in order to hold the seal-forming structure 3100 against the patient's face. The force Fₛₜᵣₐₚ may also be directed at least partially in the superior direction in order to overcome the gravitational force F_{g}. The gravitational force F_{g} may be specifically shown for the seal-forming structure 3100 and the plenum chamber 3200, but gravity would act on the entirely of the patient interface 3000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force F_{f} would act in the superior direction (e.g., against a patient's face). For example, the patient may experience the frictional force F_{f} against his lip superior (and/or other surfaces of the patient's face in contact with the seal-forming structure 3100) in order to oppose the motion in the inferior direction (which may help to stabilising the cushion in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair). In some forms the gravitational force F_{g} may also be countered by vertical components of the reaction force from the patient's face acting on the seal-forming structure 3100, for example at the nose ridge and chin regions of the patient's face, for example.

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired scaling position. The positioning and stabilising force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilising force FPSS may exceed the sum of the gravitational force F_{g} and the blowout force Fₚₗₑₙᵤₘ (with any additional positioning and stabilising force F_{PSS} being balanced by reaction force from the patient's head acting on the portions of patient interface 3000) and still maintain the seal-forming structure 3100 in an appropriate scaling position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force F_{g} and the blowout force Fₚₗₑₙᵤₘ to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force F_{PSS} necessary to achieve equilibrium.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. Suitable for a small sized head, but not a large sized head.

### 5.3.4.1 Conduit Headgear

### 5.3.4.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3Z, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3615 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one check region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a scaling position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3Z, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 7C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 3Z, each tube 3350 lies in use on a path extending from the plenum chamber 3200 across the patient's cheek region and superior to the patient's ear to the elbow 3615. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3615 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

Conduits forming part of the positioning and stabilising structure 3300, like headgear straps, may provide a force that contributes to the positioning and stabilising force F_{PSS}. As illustrated in FIG. 3Z-1, the positioning and stabilising force F_{PSS} may be the resultant force from the various forces of the different elements of the positioning and stabilising structure 3300. For example, each conduit may provide a force Fconduit directed in the posterior and respective lateral direction in order to hold the seal-forming structure 3100 against the patient's face (into the upper lip and sealing under the nose) and oppose the effect of the positive pressure in the plenum chamber 3200 to lift off the face (i.e., Fₚₗₑₙᵤₘ). The force F_{conduit} directed may also be directed at least partially in the superior direction in order to overcome the gravitational force F_{g}.

In some forms, the conduits may provide a force directed into the patient's head when the conduits are filled with pressurized air. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

The position of the patient's head may also change the gripping force of the conduits. For example, if the patient is sleeping on his side, the weight of the patient's head may compress one conduit, and the other conduit (e.g., the lateral portion not between the patient's head and a sleeping surface, like a pillow) may additionally expand in order to keep substantially the same flow rate of pressurized air.

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force F_{f} would act in the superior direction (e.g., against a patient's face). For example, the patient may experience the frictional force F_{f} against his lip superior (and/or other surfaces of the patient's face in contact with the seal-forming structure 3100) in order to oppose the motion in the inferior direction (which may help to stabilising the cushion in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair).

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired scaling position. The positioning and stabilising force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilising force F_{PSS} may exceed the sum of the gravitational force F_{g} and the blowout force Fₚₗₑₙᵤₘ (with any additional positioning and stabilising force F_{PSS} being balanced by reaction force from the patient's head acting on the portions of patient interface 3000) and still maintain the seal-forming structure 3100 in an appropriate scaling position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force F_{g} and the blowout force Fₚₗₑₙᵤₘ to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force F_{PSS} necessary to achieve equilibrium.

### 5.3.4.1.2 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3Z comprises tubes 3350, the superior portions of which comprise extendable tube sections each in the form of an extendable concertina structure 3362.

In some forms, the extendable concertina structure 3328 may be formed as a series of ridges and grooves on the surface of the tubes 3350. The concertina structure 3328 may be biased toward a retracted position, and may move to an expanded position when the patient dons the positioning and stabilising structure 3300. Because portions of the tubes 3350 may be substantially inextensible (e.g., non-extendable tube sections 3363), the concertina structures 3328 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3328. Alternatively, the tubes 3350 may be manufactured in multiple sizes (e.g., small, medium, large). The patient may select a length that most closely conforms to their head, and the concertina structures 3328 may make small adjustments in order to tailor the fit to the individual patient.

In some forms, the inlet 3332 may be disposed in the middle of the conduit 6320. For example, the tubes 3350 may be symmetric about the inlet 3332 through at least one axis.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connects to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal forming structure 3100. The tubes 3350 may contact the patient's face below the patient's cheekbones, which may be more comfortable than contact on the patient's cheekbones and may avoid excessively obscuring the patient's peripheral vision.

### 5.3.4.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3Z, the connection port 3600 is located on top of the patient's head (e.g. at a superior location with respect to the patient's head). In this example the patient interface 3000 comprises an elbow 3615 forming the connection port 3600. The elbow 3615 may be configured to fluidly connect with a conduit of an air circuit 4170. The elbow 3615 may be configured to swivel with respect to the positioning and stabilising structure 3300 to at least partially decouple the conduit from the positioning and stabilising structure 3300. In some examples the elbow 3615 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.4.1.4 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.4.2 Headgear Straps

In some forms, the positioning and stabilising structure 3300 may include headgear 3302 with at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a scaling force for the seal-forming structure 3100.

Two forms of the headgear, four-point headgear 3302-1 and two-point headgear 3302-2, are discussed in more detail below as illustrative examples.

### 5.3.4.2.1 Four-Point Connection

As shown in FIG. 7E, some forms of the headgear 3302-1 may be a four-point connection headgear. This means that the headgear 3302-1 may connect to four separate places on the plenum chamber 3200, on a frame connected to the plenum chamber 3200, and/or on arms connected to the plenum chamber 3200. The headgear 3302-1 may include four different straps providing a tensile force to help maintain the seal-forming structure 3100 in a sealing position. The positioning and stabilising structure 3300 of FIG. 3A may also be considered a four-point connection headgear.

In some forms, the headgear 3302-1 may include inferior straps 3304-1, which may connect to an inferior portion of the cushion 3050-1. The inferior straps 3304-1 may extend along the patient's cheek toward a posterior region of the patient's head. For example, the inferior straps 3304-1 may overlay the masseter muscle on either side of the patient's face. The inferior straps 3304-1 may therefore contact the patient's head below the patient's ears. The inferior straps 3304-1 may meet at the posterior of the patient's head, and may overlay the occipital bone and/or the trapezius muscle.

The headgear 3302-1 may also include superior straps 3305-1, which may overlay the temporal bones, parietal bone, and/or occipital bone. The superior straps 3305-1 may also connect to the tubes 3350 (e.g., by interfacing with the tabs 3318).

A rear strap 3307-1 may extend between the superior straps 3305-1 and between the inferior straps 3304-1. The inferior and superior straps 3304-1, 3305-1 on a given side (e.g., left or right) may also be connected to the rear strap 3307-1 adjacent to one another. The height of the rear strap 3307-1 may therefore be approximately the combined height of the inferior and superior strap 3304-1, 3305-1. The rear strap 3307-1 may overlay the occipital bone and/or the pariental bone in use. This may allow the rear strap 3307-1 to assist in anchoring the headgear 3302-1 to the patient's head.

In the illustrated example, the headgear 3302-1 may be formed with a substantially X-shape. The inferior and superior straps 3304-1, 3305-1 may be connected to a rear strap 3307-1 using stitching, ultrasonic welding, or any similar process.

In some forms, the inferior straps 3304-1 are connected to a magnetic member 3306-1. For example, each inferior straps 3304-1 may be threaded through a magnetic member 3306-1, so that a length of each inferior strap 3304-1 may be adjusted. The magnetic members 3306-1 may removably connect to the magnets 3370-1 (described below), so that the inferior straps 3304-1 may be disconnected from the plenum chamber 3200, but the length of the inferior straps 3304-1 may not be affected.

In some forms, the superior straps 3305-1 may be connected directly to the tabs 3318 of the tubes 3350. The superior straps 3305-1 may be threaded through the tabs 3318 in order to adjust the length and control the tensile force of each superior strap 3305-1.

In some forms, the headgear 3302-1 may be used only with the nose and mouth cushion 3050-1 (e.g., because the nose-only cushion 3050-1 does not have four connection points). However, the headgear 3302-1 may be used interchangeably with the tubes 3350 and the rigidiser arms 3340.

### 5.3.4.2.2 Two-Point Connection

As shown in FIG. 7F, some forms of the headgear 3302-2 may be a two-point connection headgear. This means that the headgear 3302-2 may connect to two separate places.

In some forms, the headgear 3302-2 may be formed from a continuous piece of material. In other words, the headgear 3302-2 may not be formed from multiple straps connected (e.g., stitched) together. This may be comfortable for a patient as they will not be in contact with any seams or joints connecting different straps. In other forms, the headgear 3302-2 may be formed from multiple straps (e.g., two superior straps, a rear strap, etc.) that are connected together (e.g., with stitching, ultra-sonic welding, etc.).

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3Z, the patient interface 3000 comprises a strap 3307-2 forming part of the positioning and stabilising structure 3300. The strap 3307-2 may be known as a back strap or a rear headgear strap, for example. The rear strap 3307-2 may overlay the temporal bones, parietal bone, and/or occipital bone. In other examples of the present technology, one or more further straps may be provided. For example, patient interfaces 3000 according to examples of the present technology having a nose-and-mouth cushion may have a second, lower, strap configured to lie against the patient's head proximate the patient's neck and/or against posterior surfaces of the patient's neck.

In the example shown in FIG. 3Z, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3Z, the positioning and stabilising structure 3300 comprises a pair of tabs 3318. In use a strap 3310 may be connected between the tabs 3318. The strap 3310 may be sufficiently flexible to pass around the back of the patient's head and lie comfortably against the patient's head, even when under tension in use.

As shown in FIG. 7F, some forms of the headgear 3302-2 may be at least partially bifurcated. For example, a rear strap 3307-2 of the headgear 3302-2 (e.g., configured to contact the posterior portion of the patient's head) may be wider than the surrounding portions of the headgear 3302-2. An intermediate section 3308-2 of the rear strap 3307-2 may include a slit 3309-2. A superior section of the rear strap 3307-2 may therefore be movable relative to the inferior section as a result of the slit 3309-2. This may allow the patient to have a larger strap coverage on the posterior region of their head, which may assist in better anchoring the headgear 3302-2 to the patient's head since there is no inferior strap (e.g., 3304-1).

In some forms, the headgear 3302-2 may be used only with the nasal cushion 3050-2 (e.g., because the nose and mouth cushion 3050-1 does not have four connection points). However, the headgear 3302-2 may be used interchangeably with the tubes 3350 and the rigidiser arms 3340.

### 5.3.4.3 Rigidiser Arm

As shown in FIG. 7D, a rigidiser arm 3340 may be an elongated, rigid member that assists in maintaining the cushion (e.g., the nose and mouth cushion 3050-1 or the nasal cushion 3050-2) in an operating position. The rigidiser arm 3340 may contact a side of the patient's head and provide a force to limit slipping of the seal-forming structure 3100 from the patient's nose and/or mouth.

In some forms, the rigidiser arm 3340 is constructed from a rigid material (e.g., plastic). The rigid material may not permit the rigidiser arm 3340 to stretch. Additionally, the rigidiser arm 3340 may be substantially inflexible and may be unable to bend. The rigidiser arm 3340 may be pre-molded into a desired shape in order to fit a patient's head. For example, the rigidiser arms 3340 may be molded with a curved shape to substantially correspond to the shape of the side of the patient's head (e.g., overlaying the masseter muscle and/or the temporal bone).

In certain forms, the rigidiser arm 3340 may be molded in order to conform to a specific patient's head (e.g., the rigidiser arm 3340 is customized).

In some forms, the rigidiser arm 3340 may be flexible along at least one direction. For example, the rigidiser arm 3340 may be flexible about its width and may be inflexible along its length. In other words, the rigidiser arm 3340 may be bendable about an axis along the width of the rigidiser arm 3340, but may be unable to bend about an axis perpendicular to the rigidiser arm 3340. This may allow an individual patient to adjust the rigidiser arm 3340 in order to better fit their individual head.

In certain forms, the rigidiser arm 3340 may remain in the new position after being bent. This may allow a patient adjust the shape of the rigidiser arm 3340 for their specific head and then the rigidiser arm 3340 will keep the desired shape while in use in order to promote patient comfort.

In some forms, a first end 3342 of the rigidiser arm 3340 may be a free end and a second end 3344 (e.g., opposite of the first end 3342) of the rigidiser arm 3340 may be fixed. The first end 3342 may be curved in order to minimize sharp edges that could cause patient discomfort. The first end 3342 may also overlay the patient's head proximate to the temporal bone, in use. The second end 3344 may be fixed to an arm connection structure 3504.

In some forms, the arm connection structure 3504 may be similar to the conduit connection structure 3500. For example, the arm connection structure 3504 and the conduit connection structure 3500 may have substantially the same shape. This may allow either the conduit connection structure 3500 or the arm connection structure 3504 to fit into the groove (e.g., 3266-1 or 3266-2) and connect to the plenum chamber inlet port 3254. The arm connection structure 3504 may connect to the nose and mouth cushion 3050-1 or the nose-only cushion 3050-2 in substantially the same way as the conduit connection structure 3500 (e.g., via a snap fit, press fit, friction fit, etc.).

In some forms, the arm connection structure 3504 may act as a plug for the plenum chamber inlet port 3254 (e.g., either 3254-1 and/or 3254-2). Unlike the tubes 3350, the rigidiser arm 3340 does not convey pressurized air to the plenum chamber 3200. The rigidised arm 3340 may be used with a “tube down” configuration, where a hose is connected to the vent opening 3402 (e.g., either 3402-1 and/or 3402-2), and conveys air into the plenum chamber 3200 through the vent opening 3402. In this example, air does not need to travel into or out of the plenum chamber inlet openings 3254. Thus, the arm connection structure 3504 may form a seal with the plenum chamber inlet opening 3254 in order to limit airflow into or out of the plenum chamber 3200.

### 5.3.5 Connectors for Positioning and Stabilising Structure

As best seen in FIGS. 53, 54 and 66, in examples, connectors for connecting the positioning and stabilising structure 3300 to the patent interface 3000 are provided to the fascia portion 3240. In examples the fascia portion 3240 is provided with a pair of superior headgear connector portions 3310 and a pair of inferior headgear connector portions 3320.

Each of the superior headgear connector portions 3310 comprises a buckle provided to a respective end of a band, which may be formed from textile. In other examples the superior headgear connector portions 3310 take the form of partially rigidised arms, each arm provided with a strap engagement means/component (e.g. a loop or a slot) for engaging a headgear strap at or proximate the end thereof.

In the examples shown, the inferior headgear connector portions 3320 comprise magnetic connectors, which may engage complementary connectors attached to inferior headgear straps.

As depicted in FIGS. 53, 54 and 66, the superior headgear connector portions 3310 are connected to superior headgear straps 3311 and the inferior headgear connector portions 3320 are connected to inferior headgear straps 3321.

It is to be understood that any suitable positioning and stabilising structure 3300 may be provided to patient interfaces 3000 according to examples of the present technology, and any suitable corresponding headgear connectors may be provided to the frame 3240 or cushion module.

### 5.3.6 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel. In other examples it may be located in the fascia portion 3240 or in the undercushion 3225.

As shown in FIG. 7N, a vent 3450 may be used with the patient interface 3000. The vent 3450 may have a substantially similar shape to the vent opening 3402-1 (e.g., a substantially circular shape).

The vent 3450 may be used with either the mouth and nose plenum chamber 3200-1 (e.g., illustrated in FIG. 7A) or the nose-only plenum chamber 3200-2 (e.g., illustrated in FIG. 7B).

With continued reference to FIG. 7A, the vent 3450 may include a vent housing 3404, which may be configured to engage with the vent opening 3402. The vent housing 3404 may be constructed from a rigid material or a semi-rigid material. For example, the vent housing 3404 may be constructed from plastic, metal, or any similar material. The vent housing 3404 may add rigidity to the patient interface 3000 (e.g., to limit unwanted bending that may affect the position of the seal-forming structure 3100 on the patient's face).

The vent housing 3404 may include an anterior surface 3408, a posterior surface 3412, and a groove 3416. The anterior surface 3408 faces away from the patient's face in use, and may be positioned outside the pressurized volume of the plenum chamber 3200. The posterior surface 3412 is disposed opposite to the anterior surface 3408. In use, the posterior surface 3412 may face the patient and may be disposed within the pressurized volume of the plenum chamber 3200. The groove 3416 may be formed between the anterior and posterior surfaces 3408, 3412. A portion of the plenum chamber 3200 may be received within the groove 3416 in order to retain the vent 3400 in position.

In some forms, a diffuser 3448 may be used with the vent housing 3404. The diffuser 3448 may assist with limiting the decibel output from any of the patient interface 3000 (or any other patient interface). Specifically, the diffuser 3448 may assist in limiting the decibel level associated with air output from the patient interface 3000 (e.g., exhaled air), although the diffuser 3448 may limit the decibel level of at any point in the patient interface.

In certain forms, the diffuser 3448 may diffuse, and therefore slow, the exhaust gas exiting the plenum chamber 3200 and passing through the vent housing 3404. The diffuser 3448 may assist in avoiding jetting and associated discomfort to the patient and/or bed partner (e.g., noise caused by jetting against a pillow, sheets, bedclothes, etc.).

In some forms, the diffuser may include an anterior surface 3456 that faces away from the patient in use. An outer diameter of the anterior surface 3456 may be less than an inner diameter of the vent housing 3404 proximate to the anterior surface 3408. This may form a gap 3464 through which air may travel.

### 5.3.7 Decoupling Structure(s)

In one form the patient interface 3000 includes at least one decoupling structure, for example, a swivel or a ball and socket. As shown in FIGS. 8, 9, 12, 29, 30, 37 and 38, for example, the patient interface 3000 comprises a connector 3620 structured to connect to a short tube 3610. In other examples the connector 3620 may form a connection port 3600 for attachment directly to an air circuit 4170 (e.g. to a conduit connected to an RPT device 4000). The connector 3620 may be able to move about at least one or two axes to at least partially decouple the short tube 3610 or air circuit 4170 from the cushion module 3150. In some examples the connector 3620 forms the vent 3400, for example by having a plurality of vent holes.

As described above, in some examples a fascia portion 3240 is connected to a short tube 3610 by a connector 3620, e.g. as in the example shown in FIGS. 53, 54 and 66. The connector 3620 in this example is in the form of an elbow. In other examples it may be a straight connector. The connector 3620 provides a ball and socket joint connection to the fascia portion 3240 in this example to provide for multiple axes of rotation and, in other examples, may be connected to the fascia portion so as to rotate around only a single axis (e.g. an axis coaxial with a hole in the fascia portion 3240). In such an example the connector 3620 may comprise an additional portion which rotates independently of the portion that rotates within the fascia portion 3240, to provide an additional axis of rotation available to the connection between the short tube 3610 and the fascia portion 3240. The connector 3620 may be removably attached to the fascia portion 3240. The connector 3620, by being able to rotate with respect to the fascia portion 3240 about one or more axes, forms a decoupling structure.

The short tube 3610 in the example shown in FIGS. 53, 54 and 66 also forms a decoupling structure as it decouples movement of an air circuit 4170, to which it is attached, from the fascia portion 3240, at least partially mitigating the effect of tube drag in use. Furthermore, in some examples the connection port 3600 may comprise a swivel connection to the air circuit, providing for additional or alternative decoupling of the air circuit 4170 from the fascia portion 3240.

### 5.3.8 Connection Port

Connection port 3600 allows for connection to the air circuit 4170. In the illustrated examples shown in FIGS. 8, 9, 12, 29, 30, 37 and 38, for example, the connection port 3600 is provided at a distal end of a short tube 3610. In other examples it may be provided more directly on a cushion module 3150, such as to a connector connected directly to the cushion module 3150.

In the examples shown in FIGS. 53, 54 and 66 the connection port 3600 is at a distal end of the short tube 3612. In other example the connection port 3600 may be attached to the fascia portion 3240 without a short tube 3612. In such an example the connection port 3600 may be provided by an elbow fluidly connected to the fascia portion 3240 and configured to fluidly connect to the air circuit 4170.

### 5.3.9 Forehead Support

In one form, the patient interface 3000 includes a forehead support 3700.

### 5.3.10 Anti-Asphyxia Valve

In one form, the patient interface 3000 includes an anti-asphyxia valve.

### 5.3.11 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.12 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing prospective because wider variety of patient interfaces may be created using fewer parts. Additionally or alternatively, the various combinations may allow a patient to change styles of patient interface without changing the every component.

Air may be delivered to the patient in one of two main ways. In one example, the patient may receive the flow of pressurized air through headgear tubes 3350 (see e.g., FIG. 3Z). This may be referred to as a “tube up” configuration and may position a connection port at the top of the patient's head. In other example, the patient may receive the flow of pressurized air through a conduit connected to the plenum chamber 3200, for example through the connection port 3600 (see e.g., FIG. 3A). This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 5.3.12.1 Sleeve

In some forms, to allow for modularity, a sleeve may be used with the tubes 3350 and/or the rigidiser arms 3340. The sleeve may at least partially surround the tubes 3350 and/or the rigidiser arms 3340. As shown in FIGS. 7G to 7I, different shapes of sleeves may be used, which may correspond to different types of positioning and stabilising structures 3300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use, and may feel soft against the patient's skin in order to improve patient compliance.

The material may also be flexible in order to assist in donning or doffing the sleeve from the tube 3350 or the rigidiser arms 3340. For example, the material may allow the sleeve to bend in order to conform to the shape of the tubes or conduit headgear 3350 or the rigidiser arms 3340, which may change depending on the shape of an individual patient's head.

In some forms, the sleeve may also be at least partially elastic (e.g., the material may allow the sleeve to stretch). The elastic material may help the sleeve stretch in order to fit around the tubes 3350 or the rigidiser arms 3340. The elastic material may then return to an initial position that is snug against the tubes 3350 or the rigidiser arms 3340 in order to limit the sleeve from sliding while in use.

As described in more detail below, some forms of the sleeves may be specific to a rigidising element (e.g., tubes 3350 and/or rigidiser arms 3340). However, the sleeves may assist the rigidising elements in connecting interchangeably with the version or styles of cushions (e.g., the mouth and nose cushion 3050-1, the nose-only cushion 3050-2, etc.).

### 5.3.12.1.1 Conduit Sleeve

As shown in FIG. 7G, one example of a sleeve is a conduit sleeve 3351, which may be usable with the tubes 3350 described above.

As shown in FIG. 7G, the conduit sleeve 3351 may include a curved shape that may be similar to the shape of the tubes 3350 shown in FIG. 7C. The flexible material used to construct the conduit sleeve 3351 may allow the conduit sleeve 3351 to further curve in order to correspond to the shape of the tubes 3350 (e.g., when worn by the patient).

In some forms, the conduit sleeve 3351 may include a first or superior opening 3352. The superior opening 3352 may be disposed at one end of the conduit sleeve 3351. The superior opening 3352 may be an opening to a passage that extends along at least a portion of the conduit sleeve 3351.

As shown in FIG. 7G, some forms of the conduit sleeve 3351 may also include an inferior extension 3354. The inferior extension 3354 may be positioned on an opposite end of the conduit sleeve 3351 from the superior opening 3352. The conduit sleeve 3351 may be customized to fit a particular user's face. For instance, the inferior extension 3354 of the conduit sleeve 3351 may be configured in a relatively more posterior region or anterior region of the patient's head.

Some forms of the inferior extension 3354 may include a rigid or semi-rigid piece (e.g., within the sleeve 3351). The rigid or semi-rigid piece may be constructed from a plastic material, or a similar material. Alternatively, the inferior extension 3354 may be stiffened using a manufacturing process (e.g., stitching rigidised thread, flat knitting, using thicker material).

As shown in FIG. 7G, some forms of the inferior extension 3354 may include a connection member 3356. In the illustrated example, the connection member 3356 may be a magnet, although in other examples, the connection member 3356 may be a different type of connector (e.g., a mechanical fastener, an adhesive, hook and loop material, etc.). The connection member 3356 may also be positioned at an end of the inferior extension 3354, although the connection member 3356 could alternatively be positioned anywhere along the inferior extension 3354.

In some forms, the connection member 3356 (e.g., a magnet) may be removably connected to the magnets 3370-1 of the headgear 3302-1. For example, when the conduit sleeves 3351 are connected to the tubes 3350 (see e.g., FIG. 7J), the magnets 3370-1 connected to the inferior straps 3304-1 may be removably connected to the connection member 3356 in order to provide the tensile force.

### 5.3.12.1.2 Four-Point Arm Sleeve

As shown in FIG. 7H, another example of a sleeve is a four-point arm sleeve 3380, which may be usable with the rigidiser arms 3340 described above.

As shown in FIG. 7H, the four-point arm sleeve 3380 may include a curved shape that may be similar to the shape of the rigidiser arm 3340 shown in FIG. 7D. The flexible material used to construct the four-point arm sleeve 3380 may allow the four-point arm sleeve 3380 to further curve in order to correspond to the shape of the rigidiser arm 3340 (e.g., when worn by the patient and/or went bent by the patient).

As shown in FIG. 7H, some forms of the four-point arm sleeve 3380 may include an inferior extension 3384. The inferior extension 3384 may be positioned at an end of the four-point arm sleeve 3380.

In the illustrated example, the shape and/or structure of the inferior extension 3384 is substantially the same as the shape of the inferior extension 3354. For example, the inferior extension 3384 may be more rigid as compared to the rest of the four-point arm sleeve 3380 (e.g., as a result of rigidising thread or rigid material).

As shown in FIG. 7H, some forms of the inferior extension 3384 may include a connection member 3386. In the illustrated example, the connection member 3386 may be a magnet, although in other examples, the connection member 3386 may be a different type of connector (e.g., a mechanical fastener, an adhesive, hook and loop material, etc.). The connection member 3386 may also be positioned at an end of the inferior extension 3384, although the connection member 3386 could alternatively be positioned anywhere along the inferior extension 3384.

In some forms, the connection member 3386 (e.g., a magnet) may be removably connected to the magnets 3370-1 of the headgear 3302-1. For example, when the four-point arm sleeves 3380 are connected to the rigidiser arm 3340 (see e.g., FIG. 7K), the magnets 3370-1 connected to the inferior straps 3304-1 may be removably connected to the connection member 3386 in order to provide the tensile force.

As shown in FIG. 7H, the four-point arm sleeve 3380 may include a pair of tabs 3394, which may be similar to the tab 3318 on the tubes 3350. When the four-point arm sleeve 3380 is worn by the patient, the tabs 3394 may be positioned in substantially the same place on the patient's head as where the tabs 3318 are positioned when the patient wears the tubes 3350.

### 5.3.12.1.3 Two-Point Arm Sleeve

As shown in FIG. 7I, yet another example of a sleeve is a two-point arm sleeve 3380-1, which may be usable with the rigidiser arms 3340 described above.

In some forms, the two-point arm sleeve 3380-1 may be similar to the four-point arm sleeve 3380 described above. Only some similarities and differences may be described below.

As shown in FIG. 7I, the two-point arm sleeve 3380-1 may include an inferior opening 3388-1 that is positioned at an end of the two-point arm sleeve 3380-1. The inferior opening 3388-1 may form an opening to a passageway through the two-point arm sleeve 3380-1. In the illustrated example, the inferior opening 3388-1 may open into a surface of the conduit sleeve 3380-1.

As shown in FIG. 7I, the two-point arm sleeve 3380-1 may include a pair of tabs 3394-1, which may be similar to the tab 3318 on the tubes 3350. When the two-point arm sleeve 3380-1 is worn by the patient, the tabs 3394-1 may be positioned in substantially the same place on the patient's head as where the tabs 3318 are positioned when the patient wears the tubes 3350.

### 5.3.12.2 Assembled Patient Interfaces

As illustrated in FIGS. 7J to 7M, the various elements described above may be combined into four different patient interfaces. The different patient interfaces may allow patients to use different styles based on their individual comfort. The modularity of the different elements (e.g., the ability to be used in multiple styles of patient interfaces) may simplify manufacturing and/or may allow a patient to more easily switch between styles of patient interfaces.

### 5.3.12.2.1 Nose and Mouth Mask Tube Up Configuration

As illustrated in FIG. 7J, the patient may wear the cushion 3050-1 in a tube-up configuration with the tubes 3350 and the four-point headgear 3302-1. This assembly may form a tube up nose and mouth patient interface 3000-1.

In some forms, a conduit sleeve may be used with the tubes 3350 in order to enable a patient to experience the “tube up” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the tubes 3350 of the positioning and stabilising structure 3300. The tubes 3350 (via the conduit connection structure 3500), may be used to connect the tubes 3350 to the cushion 3050-1. The conduit sleeves provide the magnets in order to connect to the magnets 3370-1 (see e.g., FIG. 7E) of the four-point headgear 3302-1. Alternatively, a different connection form may be used.

As illustrated in FIG. 7J, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.12.2.2 Nose and Mouth Mask Tube Down Configuration

As illustrated in FIG. 7K, the patient may wear the cushion 3050-1 in a tube-down configuration with the rigidiser arms 3340 and the four-point headgear 3302-1. This assembly may form a tube down nose and mouth patient interface 3000-2.

In some forms, a conduit sleeve may be used with the rigidiser arms 3340 in order to enable a patient to experience the “tube down” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the rigidiser arms 3340 of the positioning and stabilising structure 3300. The rigidiser arms 3340 (via the conduit connection structure 3504), may be used to connect the rigidiser arms 3340 to the cushion 3050-1. The conduit sleeves provide the magnets in order to connect to the magnets 3370-1 (see e.g., FIG. 7E) of the four-point headgear 3302-1. Alternatively, a different connection form may be used.

As illustrated in FIG. 7K, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.12.2.3 Nose Mask Tube Up Configuration

As illustrated in FIG. 7L, the patient may wear the cushion 3050-2 in a tube-up configuration with the tubes 3350 and the two-point headgear 3302-2. This assembly may form a tube up nose only patient interface 3000-3

A conduit sleeve may be used with the tubes 3350, and may provide additional comfort to the patient. The sleeve may not add additional connection points to connect the positioning and stabilising structure 3300 on the cushion 3050-2. In the illustrated example, the tubes 3350 of the positioning and stabilising structure 3300 may be connected directly to the cushion 3050-2.

As illustrated in FIG. 7L, the two-point headgear 3302-2 may connect to the tabs 3318 on the tubes 3350 in order to provide a tensile force that maintains the cushion 3050-2 in a sealing position on the patient's head.

### 5.3.12.2.4 Nose Mask Tube Down Configuration

As illustrated in FIG. 7M, the patient may wear the cushion 3050-2 in a tube-up configuration with the rigidiser arms 3340 and the two-point headgear 3302-2. This assembly may form a tube down nose only patient interface 3000-4.

A conduit sleeve may be used with the rigidiser arms 3340, and may provide additional comfort to the patient. The sleeve may not add additional connection points to connect the positioning and stabilising structure 3300 on the cushion 3050-2. In the illustrated example, the rigidiser arms 3340 of the positioning and stabilising structure 3300 may be connected directly to the cushion 3050-2.

As illustrated in FIG. 7M, the two-point headgear 3302-2 may connect to the tabs 3318 on the sleeve in order to provide a tensile force that maintains the cushion 3050-2 in a sealing position on the patient's head.

### 5.3.12.2.5 Modularity of Elements

FIG. 7P illustrates how the different elements can be combined in order to form the four different patient interfaces described above. As illustrated, the different components may be reused for different styles of patient interfaces. This may allow for easier manufacturing and assembly, because a large number of the same components may be produced and used in a variety of styles. The only components not used in multiple styles may be the sleeves. However, the sleeves may be easier to manufacture. FIG. 7O shows a portion of air circuit 4170 that may interface with the patient interface, while FIG. 7N shows a vent 3404 that may interchangeably replace the air circuit shown in FIG. 7O, depending on the style of the patient interface.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., one or more air filters 4110 such as an inlet air filter 4112 and/or an outlet air filter 4114, an inlet muffler 4122, a pressure generator 4140 capable of supplying air at positive pressure (e.g., a blower 4142, comprising a motor 4144), one or more mufflers 4120 such as an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016. An anti spillback valve 4160 may be provided between the pneumatic block 4020 and the humidifier 5000.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more transducers 4270 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Algorithms

As mentioned above, in some forms of the present technology, the central controller 4230 may be configured to implement one or more algorithms expressed as computer programs stored in a non-transitory computer readable storage medium, such as memory. The algorithms are generally grouped into groups referred to as modules.

In other forms of the present technology, some portion or all of the algorithms may be implemented by a controller of an external device such as the local external device or the remote external device. In such forms, data representing the input signals and/or intermediate algorithm outputs necessary for the portion of the algorithms to be executed at the external device may be communicated to the external device via the local external communication network or the remote external communication network. In such forms, the portion of the algorithms to be executed at the external device may be expressed as computer programs stored in a non-transitory computer readable storage medium accessible to the controller of the external device. Such programs configure the controller of the external device to execute the portion of the algorithms.

In such forms, the therapy parameters generated by the external device via the therapy engine module (if such forms part of the portion of the algorithms executed by the external device) may be communicated to the central controller to be passed to the therapy control module.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. The reservoir 5110 comprises a conductive portion 5120 configured to allow efficient transfer of heat from the heating element 5240 to the volume of liquid in the reservoir 5110. The reservoir 5110 may comprise a water level indicator 5150.

In some arrangements, the humidifier reservoir dock 5130 may comprise a locking feature such as a locking lever 5135 configured to retain the reservoir 5110 in the humidifier reservoir dock 5130.

### 5.7 Breathing Waveforms

FIG. 6 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak-0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Respiratory Therapy Modes

Various respiratory therapy modes may be implemented by the disclosed respiratory therapy system including CPAP therapy, Bi-level therapy and/or High Flow therapy.

### 5.9 Glossary

For the purposes of the present technology disclosure, in certain forms of the present technology, one or more of the following definitions may apply. In other forms of the present technology, alternative definitions may apply.

### 5.9.1 General

Air: In certain forms of the present technology, air may be taken to mean atmospheric air, and in other forms of the present technology air may be taken to mean some other combination of breathable gases, e.g. oxygen enriched air.
Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.
Automatic Positive Airway Pressure (APAP) therapy: CPAP therapy in which the treatment pressure is automatically adjustable, e.g. from breath to breath, between minimum and maximum limits, depending on the presence or absence of indications of SDB events.
Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.
Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.
Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.
Leak: The word leak will be taken to be an unintended flow of air. In one example, leak may occur as the result of an incomplete seal between a mask and a patient's face. In another example leak may occur in a swivel elbow to the ambient.
Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.
Noise, radiated (acoustic): Radiated noise in the present document refers to noise which is carried to the patient by the ambient air. In one form, radiated noise may be quantified by measuring sound power/pressure levels of the object in question according to ISO 3744.
Noise, vent (acoustic): Vent noise in the present document refers to noise which is generated by the flow of air through any vents such as vent holes of the patient interface.
Patient: A person, whether or not they are suffering from a respiratory condition.
Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O. The pressure in the patient interface is given the symbol Pm, while the treatment pressure, which represents a target value to be achieved by the interface pressure Pm at the current instant of time, is given the symbol Pt.
Respiratory Pressure Therapy: The application of a supply of air to an entrance to the airways at a treatment pressure that is typically positive with respect to atmosphere.
Ventilator: A mechanical device that provides pressure support to a patient to perform some or all of the work of breathing.
   5.9.1.1 Materials & their Properties
Hardness: Refers to durometer or indentation hardness, which is a material property measured by indentation of an indentor (e.g., as measured in accordance with ASTM D2240).
   ‘Soft’ materials may include silicone or thermo-plastic elastomer (TPE), and may, e.g. readily deform under finger pressure.
   ‘Hard’ materials may include polycarbonate, polypropylene, and may not e.g. readily deform under finger pressure.
Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.
Polycarbonate: a thermoplastic polymer of Bisphenol-A Carbonate.

### 5.9.1.2 Mechanics

Axes:
   a. Neutral axis: An axis in the cross-section of a beam or plate along which there are no longitudinal stresses or strains.
   b. Longitudinal axis: An axis extending along the length of a shape. The axis generally passes through a center of the shape.
   c. Circumferential axis: An axis oriented perpendicularly with respect to the longitudinal axis. The axis may be specifically present in pipes, tubes, cylinders, or similar shapes with a circular and/or elliptical cross section.
Deformation: The process where the original geometry of a member changes when subjected to forces, e.g. a force in a direction with respect to an axis. The process may include stretching or compressing, bending and, twisting.
Elasticity: The ability of a material to return to its original geometry after deformation.
Floppy structure or component: A structure or component that will change shape, e.g. bend, when caused to support its own weight, within a relatively short period of time such as 1 second.
Resilience: Ability of a material to absorb energy when deformed elastically and to release the energy upon unloading.
Resilient: Will release substantially all of the energy when unloaded. Includes e.g. certain silicones, and thermoplastic elastomers.
Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.
Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.
Viscous: The ability of a material to resist flow.
Visco-elasticity: The ability of a material to display both elastic and viscous behaviour in deformation.
Yield: The situation when a material can no longer return back to its original geometry after deformation.

### 5.9.1.3 Structural Elements

Compression member: A structural element that resists compression forces.
Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.
Frame: Frame will be taken to mean a mask structure that bears the load of tension between two or more points of connection with a headgear. A mask frame may be a non-airtight load bearing structure in the mask. However, some forms of mask frame may also be air-tight.
Membrane: Membrane will be taken to mean a typically thin element that has, preferably, substantially no resistance to bending, but has resistance to being stretched.
Tie (noun): A structure designed to resist tension.

### Thin Structures:

a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

### Thick Structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.
Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.
Stiffener: A stiffener will be taken to mean a structural component designed to increase the bending resistance of another component in at least one direction.
Strut: A strut will be taken to be a structural component designed to increase the compression resistance of another component in at least one direction.
Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.9.2 Anatomy

### 5.9.2.1 Anatomy of the Face

Ala: the external outer wall or “wing” of each nostril (plural: alar)
Alar angle: An angle formed between the ala of each nostril.
Alare: The most lateral point on the nasal ala.
Alar curvature (or alar crest) point: The most posterior point in the curved base line of each ala, found in the crease formed by the union of the ala with the cheek.
Auricle: The whole external visible part of the ear.
(nose) Bony framework: The bony framework of the nose comprises the nasal bones, the frontal process of the maxillae and the nasal part of the frontal bone.
(nose) Cartilaginous framework: The cartilaginous framework of the nose comprises the septal, lateral, major and minor cartilages.
Columella: the strip of skin that separates the nares and which runs from the pronasale to the upper lip.
Columella angle: The angle between the line drawn through the midpoint of the nostril aperture and a line drawn perpendicular to the Frankfort horizontal while intersecting subnasale.
Frankfort horizontal plane: A line extending from the most inferior point of the orbital margin to the left tragion. The tragion is the deepest point in the notch superior to the tragus of the auricle.
Glabella: Located on the soft tissue, the most prominent point in the midsagittal plane of the forehead.
Lateral nasal cartilage: A generally triangular plate of cartilage. Its superior margin is attached to the nasal bone and frontal process of the maxilla, and its inferior margin is connected to the greater alar cartilage.
Lip, lower (labrale inferius): The lip extending between the subnasale and the mouth.
Lip, upper (labrale superius): The lip extending between the mouth and the supramenton.
Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.
Nares (Nostrils): Approximately ellipsoidal apertures forming the entrance to the nasal cavity. The singular form of nares is naris (nostril). The nares are separated by the nasal septum.
Naso-labial sulcus or Naso-labial fold: The skin fold or groove that runs from each side of the nose to the corners of the mouth, separating the cheeks from the upper lip.
Naso-labial angle: The angle between the columella and the upper lip, while intersecting subnasale.
Otobasion inferior: The lowest point of attachment of the auricle to the skin of the face.
Otobasion superior: The highest point of attachment of the auricle to the skin of the face.
Pronasale: the most protruded point or tip of the nose, which can be identified in lateral view of the rest of the portion of the head.
Philtrum: the midline groove that runs from lower border of the nasal septum to the top of the lip in the upper lip region.
Pogonion: Located on the soft tissue, the most anterior midpoint of the chin.
Ridge (nasal): The nasal ridge is the midline prominence of the nose, extending from the Sellion to the Pronasale.
Sagittal plane: A vertical plane that passes from anterior (front) to posterior (rear). The midsagittal plane is a sagittal plane that divides the body into right and left halves.
Sellion: Located on the soft tissue, the most concave point overlying the area of the frontonasal suture.
Septal cartilage (nasal): The nasal septal cartilage forms part of the septum and divides the front part of the nasal cavity.
Subalare: The point at the lower margin of the alar base, where the alar base joins with the skin of the superior (upper) lip.
Subnasal point: Located on the soft tissue, the point at which the columella merges with the upper lip in the midsagittal plane.
Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.
Mandible: The mandible forms the lower jaw. The mental protuberance is the bony protuberance of the jaw that forms the chin.
Maxilla: The maxilla forms the upper jaw and is located above the mandible and below the orbits. The frontal process of the maxilla projects upwards by the side of the nose, and forms part of its lateral boundary.
Nasal bones: The nasal bones are two small oblong bones, varying in size and form in different individuals; they are placed side by side at the middle and upper part of the face, and form, by their junction, the “bridge” of the nose.
Nasion: The intersection of the frontal bone and the two nasal bones, a depressed area directly between the eyes and superior to the bridge of the nose.
Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.
Orbit: The bony cavity in the skull to contain the eyeball.
Parietal bones: The parietal bones are the bones that, when joined together, form the roof and sides of the cranium.
Temporal bones: The temporal bones are situated on the bases and sides of the skull, and support that part of the face known as the temple.
Zygomatic bones: The face includes two zygomatic bones, located in the upper and lateral parts of the face and forming the prominence of the cheek.

### 5.9.3 Patient Interface

Anti-asphyxia valve (AAV): The component or sub-assembly of a mask system that, by opening to atmosphere in a failsafe manner, reduces the risk of excessive CO2 rebreathing by a patient.
Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.
Frame: Frame will be taken to mean a mask structure that bears the load of tension between two or more points of connection with a headgear. A mask frame may be a non-airtight load bearing structure in the mask. However, some forms of mask frame may also be air-tight.
Headgear: Headgear will be taken to mean a form of positioning and stabilising structure designed to hold a device, e.g., a mask, on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.
Membrane: Membrane will be taken to mean a typically thin element that has, preferably, substantially no resistance to bending, but has resistance to being stretched.
Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.
Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.
Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.
Stiffener: A stiffener will be taken to mean a structural component designed to increase the bending resistance of another component in at least one direction.
Strut: A strut will be taken to be a structural component designed to increase the compression resistance of another component in at least one direction.
Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.
Tie (noun): A structure designed to resist tension.
Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.9.4 Shape of Structures

### 5.9.4.1 Curvature in One Dimension

The curvature of a plane curve at p may be described as having a sign (e.g. positive, negative) and a magnitude (e.g. 1/radius of a circle that just touches the curve at p).
Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.
Zero curvature: If the curve at p is a straight line, the curvature will be taken to be zero (if the imaginary small person leaves the point p, they can walk on a level, neither up nor down). See FIG. 3D.
Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.9.4.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.
Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.
Region of a surface: A connected set of points on a surface. The set of points in a region may have similar characteristics, e.g. curvatures or signs.
Saddle region: A region where at each point, the principal curvatures have opposite signs, that is, one is positive, and the other is negative (depending on the direction to which the imaginary person turns, they may walk uphill or downhill).
Dome region: A region where at each point the principal curvatures have the same sign, e.g. both positive (a “concave dome”) or both negative (a “convex dome”).
Cylindrical region: A region where one principal curvature is zero (or, for example, zero within manufacturing tolerances) and the other principal curvature is non-zero.
Planar region: A region of a surface where both of the principal curvatures are zero (or, for example, zero within manufacturing tolerances).

### 5.9.4.3 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.11 Selected Reference Signs List

| | |
|---|---|
| patient | 1000 |
| bed partner | 1100 |
| patient interface | 3000 |
| nose and mouth patient interface | 3000-1 |
| nose and mouth patient interface | 3000-2 |
| nose-only patient interface | 3000-3 |
| nose-only patient interface | 3000-4 |
| mouth cushion | 3050-1 |
| nose cushion | 3050-2 |
| seal - forming structure | 3100 |
| cushion module | 3150 |
| plenum chamber | 3200 |
| plenum chamber | 3200-1 |
| plenum chamber | 3200-2 |
| plenum chamber inlet port | 3202 |
| chord | 3210 |
| superior point | 3215 |
| membrane portion | 3220 |
| undercushion | 3225 |
| inwardly facing wall | 3226 |
| posterior support portions | 3227 |
| posterior facing surface | 3227a |
| medially facing surface | 3227b |
| nasal recess | 3228 |
| superiorly facing surface | 3229 |
| medial recess | 3229a |
| inferior point | 3230 |
| medial peripheral portion | 3231 |
| lateral peripheral portions | 3232 |
| channel | 3233 |
| hinge regions | 3234 |
| fascia portion | 3240 |
| plenum chamber inlet port | 3254 |
| plenum chamber inlet port | 3254-1 |
| plenum chamber inlet port | 3254-2 |
| groove | 3266-1 |
| groove | 3266-2 |
| positioning and stabilising structure | 3300 |
| headgear | 3302 |
| headgear | 3302-1 |
| headgear | 3302-2 |
| inferior strap | 3304-1 |
| inferior strap | 3304-2 |
| superior strap | 3305-1 |
| magnetic member | 3306-1 |
| rear strap | 3307-1 |
| rear strap | 3307-2 |
| intermediate section | 3308-2 |
| slit | 3309-2 |
| superior headgear connector portions | 3310 |
| superior headgear straps | 3312 |
| strap | 3312 |
| joint | 3315 |
| tab | 3318 |
| inferior headgear connector portions | 3320 |
| inferior headgear straps | 3321 |
| concertina structure | 3328 |
| inlet | 3332 |
| rigidiser arm | 3340 |
| first end | 3342 |
| second end | 3344 |
| tube | 3350 |
| sleeve | 3351 |
| superior opening | 3352 |
| inferior extension | 3354 |
| connection member | 3356 |
| non - extendable tube sections | 3363 |
| magnet | 3370-1 |
| four - point arm sleeve | 3380 |
| conduit sleeve | 3380-1 |
| inferior extension | 3384 |
| connection member | 3386 |
| inferior opening | 3388-1 |
| tabs | 3394 |
| tabs | 3394-1 |
| vent | 3400 |
| vent opening | 3402 |
| vent opening | 3402-1 |
| vent opening | 3402-2 |
| vent housing | 3404 |
| anterior surface | 3408 |
| posterior surface | 3412 |
| groove | 3416 |
| diffuser | 3448 |
| vent | 3450 |
| conduit connection structure | 3500 |
| arm connection structure | 3504 |
| connection port | 3600 |
| short tube | 3610 |
| elbow | 3615 |
| swivel | 3620 |
| forehead support | 3700 |
| ISO | 3744 |
| unsealed patient interface | 3800 |
| prongs | 3810a |
| prongs | 3810b |
| lumens | 3820a |
| lumens | 3820b |
| RPT device | 4000 |
| external housing | 4010 |
| upper portion | 4012 |
| portion | 4014 |
| panel | 4015 |
| chassis | 4016 |
| handle | 4018 |
| pneumatic block | 4020 |
| air filter | 4110 |
| inlet air filter | 4112 |
| outlet air filter | 4114 |
| muffler | 4120 |
| inlet muffler | 4122 |
| outlet muffler | 4124 |
| pressure generator | 4140 |
| blower | 4142 |
| motor | 4144 |
| anti - spill back valve | 4160 |
| air circuit | 4170 |
| air circuit | 4171 |
| supplementary gas | 4180 |
| electrical components | 4200 |
| single Printed Circuit Board Assembly PCBA | 4202 |
| power supply | 4210 |
| input device | 4220 |
| transducer | 4270 |
| humidifier | 5000 |
| humidifier inlet | 5002 |
| humidifier outlet | 5004 |
| humidifier base | 5006 |
| reservoir | 5110 |
| conductive portion | 5120 |
| humidifier reservoir dock | 5130 |
| locking lever | 5135 |
| water level indicator | 5150 |
| heating element | 5240 |

1. A cushion module for a patient interface, the cushion module comprising:
a plenum chamber pressurisable to a therapeutic pressure of at least 4 cmH2O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a fascia portion at least partially forming an anterior side of the cushion module, the seal-forming structure being attached to the fascia portion;
wherein the fascia portion comprises a curved shape and is curved at least partially towards a posterior direction on lateral sides of the fascia portion in use, and wherein the fascia portion is biased away from the curved shape towards a flatter shape than the curved shape to provide tautness to the seal-forming structure in use.

2. The cushion module of claim 1, wherein the fascia portion is first formed into a formed shape having less curvature than the curved shape, and wherein after attachment of the seal-forming structure to the fascia portion the seal-forming structure holds the fascia portion in the curved shape, and the fascia portion is biased away from the curved shape by a tendency to return to the formed shape.

3. The cushion module of claim 2, wherein the fascia portion is thermoformed into the formed shape.

4. The cushion module of claim 1, wherein the cushion module comprises a resilient member biasing the fascia portion away from the curved shape.

5. The cushion module of claim 4, wherein the resilient member is provided interior to the plenum chamber.

6. The cushion module of claim 5, wherein the resilient member engages the fascia portion along a connection between the fascia portion and the seal-forming structure.

7. The cushion module of claim 4, wherein the resilient member is biased towards an annular shape.

8. The cushion module of claim 7, wherein the resilient member is formed in the annular shape and deformed during provision to the interior of the plenum chamber, the resilient member being biased towards the annular shape in use to bias the fascia portion away from the curved shape in use.

9. The cushion module of claim 4, wherein the resilient member is formed from a polymer material.

10. The cushion module of claim 4, wherein the resilient member is moulded.

11. The cushion module of claim 1, wherein the fascia portion at least partially forms the plenum chamber.

12. The cushion module of claim 1, wherein the fascia portion is formed at least partially from one or more foam and/or textile materials.

13. The cushion module of claim 12, wherein the fascia portion is formed from foam covered with a textile material on at least a non-patient facing side thereof.

14. The cushion module of claim 1, wherein the seal-forming structure comprises a membrane portion configured to engage the patient's face.

15. The cushion module of claim 1, wherein the membrane portion comprises a nasal hole through which air can flow to both the patient's nares, in use.

16. The cushion module of claim 1, wherein the membrane portion comprises an oral hole through which air can flow to the patient's mouth, in use.

17. The cushion module of claim 1, wherein the membrane portion is at least partially formed from a textile material and is air-impermeable.

18. The cushion module of claim 1, wherein the cushion module comprises at least one pair of headgear connection portions connected to the fascia portion and configured to connect to a positioning and stabilising structure.

19. The cushion module of claim 18, wherein the at least one pair of headgear connection portions comprises a pair of superior headgear connection portions connected to the fascia portion and a pair of inferior headgear connection portions connected to the fascia portion.
